(19) United States
(12) Patent Application Publication
(10) Pub. No.: US 2020/0348300 A1
(43) Pub. Date: Nov. 5, 2020

(54) MYCOBACTERIUM AVIUM SUBSPECIES PARATUBERCULOSIS IMMUNODIAGNOSTIC ANTIGENS, METHODS, AND KITS COMPRISING SAME

(71) Applicants: THE PENN STATE RESEARCH FOUNDATION, University Park, PA (US); IMMPORT THERAPEUTICS, INC., d/b/a ANTIGEN DISCOVERY, INC., Irvine, CA (US); CORNELL UNIVERSITY, Ithaca, NY (US); THE UNITED STATES OF AMERICA, AS REPRESENTED BY THE SECRETARY OF AGRICULTURE, Washington, DC (US)

(72) Inventors: Vivek Kapur, Bellefonte, PA (US); Lingling Li, University Park, PA (US); Joseph J. Campo, Irvine, CA (US); Yrjo T. Grohn, Ithaca, NY (US); Bettina Wagner, Ithaca, NY (US); John P. Bannantine, Ames, IA (US); Judith R. Stabel, Ames, IA (US)

(21) Appl. No.: 15/733,387

(22) PCT Filed: Jan. 18, 2019
(86) PCT No.: PCT/US19/14128

§ 371 Date: Jul. 17, 2020

Related U.S. Application Data
(60) Provisional application No. 62/618,891, filed on Jan. 18, 2018.

(51) Int. Cl. G01N 33/569; G01N 33/543

(52) U.S. Cl. CPC G01N 33/5695; G01N 2800/60; G01N 33/54306

(57) ABSTRACT

The present invention provides novel Mycobacterium avium subspecies paratuberculsis (MAP) derived antigens which may be used to diagnose and thereafter effectively treat animals that have been infected with MAP, Further provided are methods of determining whether an animal is infected with MAP, and methods of diagnosing and treating Johne's disease. The invention also relates to a kit for the implementation of the methods.

### CROSS-REFERENCE TO RELATED APPLICATIONS

This application claims priority under 35 U.S.C. § 119 to provisional application Ser. No. 62/618,891 filed Jan. 18, 2018, herein incorporated by reference in its entirety.

### GRANT REFERENCE

This invention was made with government support under Grant No. 2015-67015-23177 and under Hatch Act Project No. PEN04512 awarded by the United States Department of Agriculture. The Government has certain rights in the invention.

### FIELD OF THE INVENTION

The present invention concerns novel antigens derived from Mycobacterium avium subspecies paratuberculsis (MAP), their use in the diagnosis of both clinical and subclinical MAP infected subjects, and corresponding methods of use and kits.

### BACKGROUND OF THE INVENTION

A slow-growing bacterium, Mycobacterium avium subspecies paratuberculosis (MAP) is the causative agent of Johne's disease (JD) in cattle. JD has a high prevalence rate and results in considerable adverse impact on animal health and productivity in the US. Progress in controlling the spread of infection has been impeded by the lack of reliable diagnostic tests that can identify animals early in the infection process and help break the transmission chain. The development of rapid, sensitive, and specific assays to identify infected animals is essential to the formulation of rational strategies to control the spread of MAP.

In 1996, the National Animal Health Monitoring System conducted a survey of dairy farms using serological analysis to determine the prevalence of Johne's disease in the U.S. The results of that study showed an estimated 20-40% of surveyed herds have some level of MAP. Furthermore, it is estimated that annual losses in the U.S. from MAP in cattle herds may exceed $220 million.

The pathogenesis of MAP has been recently reviewed by Harris and Barletta (2001, Clin. Microbiol. Rev., 14:489-512). Cattle become infected with MAP as calves but often do not develop clinical signs until 2 to 5 years of age. The primary route of infection is through ingestion of fecal material, milk or colostrum containing MAP microorganisms. Epithelial M cells likely serve as the port of entry for MAP into the lymphatic system similar to other intracellular pathogens such as salmonella. MAP survive and may even replicate within macrophages in the wall of the intestine and in regional lymph nodes. After an incubation period of several years, extensive granulomatous inflammation occurs in the terminal small intestine, which leads to malabsorption and protein-losing enteropathy. Cattle shed minimal amounts of MAP in their feces during the subclinical phase of infection, and yet over time, this shedding can lead to significant contamination of the environment and an insidious spread of infection throughout the herd before the animal is diagnosed. During the clinical phase of infection, fecal shedding of the pathogen is high and can exceed 10¹⁰ organisms/g of feces. The terminal clinical stage of disease is characterized by chronic diarrhea, rapid weight loss, diffuse edema, decreased milk production, and infertility. Although transmission of MAP occurs primarily through the fecal-oral route, it has also been isolated from reproductive organs of infected males and females.

It is an object of the invention to provide novel antigens which may be used to diagnose and thereafter effectively treat diagnosed animals that have been infected with MAP.

It is a further object of the present invention to provide a kit, method and device for detecting infection with MAP at clinical or subclinical stages and which has improved reliability compared with methods of the prior art. It is also desirable to find a method, kit or device which can reliably distinguish subclinical infection. Other objects, advantages and features of the present invention will become apparent from the following specification taken in conjunction with the accompanying examples or drawings.

### SUMMARY OF THE INVENTION

According to a first aspect of the present invention, there is provided a method of determining whether an individual is infected with Alycobacterium avium subspecies paratuberculosis (MAP), the method comprising obtaining a sample from the animal and detecting the presence or absence of the binding of a biomarker in the sample with one or more MAP derived antigens. In some embodiments, the method further comprises treating the animal to kill or deactivate MAP bacteria to ameliorate the symptoms of or prevent the onset of Johne's disease if the presence of the biomarker is detected in the sample.

Preferably the present invention provides a method of determining whether an individual is infected with MAP. The method may involve detection of a biomarker in the sample that is indicative of infection with MAP. In some embodiments the method may involve detecting a biomarker which is indicative of infection with MAP but which does not necessarily mean the individual has an active disease. For example, the present invention may provide a method of detecting the presence of a MAP infection at subclinical levels. In some embodiments, the biomarker is an antibody indicative of infection with MAP. In certain embodiments, the detecting is accomplished by ELISA, a multiplex bead-based immunoassay format, and/or flow cytometry.

The present invention preferably relates to a method of determining the presence in a sample of an antibody indicative of infection with or exposure to MAP. Further provided is a method of detecting antibodies which are associated with MAP in a biological sample, the method comprising contacting the sample with one or more MAP derived antigens and detecting the binding the antigens with an antibody in the sample. The sample may be taken from any individual suspected of infection with MAP. In preferred embodiments the individual is a mammal. It may be a ruminant, for example, a cow. In some preferred embodiments the individual is a human. In some embodiments, the sample is serum or milk.

Further provided is a method of diagnosing and treating Johne's disease, the method comprising obtaining a sample from an animal, detecting the presence or absence of the binding of a biomarker in the sample with one or more MAP derived antigens; and treating an animal with the presence of said biomarker to kill or deactivate MAP bacteria to ameliorate the symptoms of or prevent the onset of Johne's disease.

Applicants have identified several novel antigens from MAP which are predictive of the presence of infection by MAP. The specificity of these antigens for detection is very high and when used together infection can be detected at very low levels. Applicants have further identified combinations of four, five, or six antigens which when used together as an assay can be highly predictive. In some embodiments, the antigens are one or more of MAP1272c, MAP1569, MAP2121c, MAP2942c, MAP2609, and MAP1201c+2942c. In some embodiments, the antigens are MAP1272c, MAP1569, MAP2942c, and MAP2609. In some embodiments, the antigens are MAP1272c, MAP1569, MAP2121c, MAP2942c, and MAP2609. In another embodiment, the antigens are MAP1272c, MAP1569, MAP2121c, MAP2942c, MAP2609, and MAP1201c+2942c. In yet another embodiment, the antigen comprises one or more immunogenic fragments of the MAP derived antigens.

In certain embodiments, the antigen comprises one or more immunogenic fragments of MAP1569, MAP2609, and/or MAP2942c.

In other embodiments, the antigens are one or more of MAP0019c, MAP0117, MAP0123, MAP0357, MAP0433c, MAP0616c, MAP0646c, MAP0858, MAP0953, MAP1152, MAP1224c, MAP1298, MAP1506, MAP1525, MAP1561c, MAP1651c, MAP1761c, MAP1782c, MAP1960, MAP1968c, MAP1986, MAP2093c, MAP2100, MAP2117c, MAP2158, MAP2187c, MAP2195, MAP2288c, MAP2447c, MAP2497c, MAP2694, MAP2875, MAP3039c, MAP3305c, MAP3527, MAP3531c, MAP3540c, MAP3762c, MAP3773c, MAP3852c, MAP4074, MAP4143, MAP4225c, MAP4231, and MAP4339.

Further provided is a kit for determining the presence or absence of a biomarker in a sample. In certain embodiments, the kit comprises one or more of the MAP derived antigens and means for detecting the binding of the antigen with a biomarker present within a sample.

### BRIEF DESCRIPTION OF THE DRAWINGS

The following figures are included to illustrate certain aspects of the present invention, and should not be viewed as exclusive embodiments. The subject matter disclosed is capable of considerable modifications, alterations, combinations, and equivalents in form and function, as will occur to those skilled in the art and having the benefit of this disclosure.

FIGS. 1A-1B show highly reactive proteins identified in MTB microarray. FIG. 1A is a Venn diagram at 10% threshold shows antigen hit number distribution of all 4 groups: negative low exposure (NL), negative high exposure (NH), fecal positive & ELISA negative (F+E−), and fecal positive & ELISA positive (F+E+). The four ellipses show the total number of hits from four groups and majority antigens are shared among 4 groups. The non-overlapping parts of the 4 ellipses represent the unique antigens for each group. FIG. 1B shows serological reactivity in selected groups. Normalized mean intensities in each group on unique and shared antigens. The height of bars represents the mean intensity. Standard error bars are added to each column.

FIGS. 2A-2B show different profiles of comparison of infected groups with NL and NH as a reference. FIG. 2A shows number of significantly reactive proteins identified in F+E− group in comparison with NL and NH. The large circle represents the number of significantly reactive proteins in comparison with NL and the smaller circle represents the number of identified proteins in comparison with NH. The overlap part represents the number of proteins shared. FIG. 2B shows number of significantly reactive proteins identified in F+E+ group in comparison with NL and NH.

FIG. 3 shows proteins identified in NH, F+E−, and F+E+ group. Unique proteins represent significantly reactive (P<0.05) proteins identified only in the specific group (NH, F+E−, or F+E+). Shared proteins represent significantly reactive (P<0.05) proteins identified in two or three groups.

FIGS. 4A-4F show patterns of mean intensities in each group. The square markers represent mean intensities significantly higher than that in NL and triangle markers indicate mean intensities significantly lower than that of NL. Standard error bars are added to each spot. FIG. 4A shows NL only. FIG. 4B shows in NH only. FIG. 4C shows F+E− only. FIG. 4D shows F+E+. FIG. 4E shows F+E− and F+E+. FIG. 4F shows all three groups including NH, F+E−, and F+E+. Mean intensities are significantly higher in NL in NH; in F+E−; in F+E+; in F+E− and F+E+; in all three groups including NH, F+E−, and F+E+ (Y axis: mean intensity of each group).

FIGS. 5A-5C show a comparison of MAP3939c with 5 MTB orthologues. FIG. 5A shows a multiple alignment of MAP3939c with 5 MTB orthologue (SEQ ID NOs:101-106). As shown in the alignment, there is the highest identity between MAP3939c and Rv0442c. FIGS. 5B-5C show similar structure characters between MAP3939c and Rv0442c (Protean of Lasergene, DNAstar, Madison, Wis.).

FIG. 6 shows patterns of serum reactivity to MTB proteins with their odds ratios that differed significantly in at least one of the 4 groups. The heatmap shows the odds ratio of the serum reactivity from 4 groups to each of the 47 proteins. Each column represents one protein, odds ratios (rows) are visualized as a color spectrum. The heatmap was generated using the ComplexHeatmap package in R. The clustering was performed using the pvclust package with multiscale bootstrap resampling. Arguments passed to the pvclust command for the hierarchical clustering method (method.hclust) was “median” and for the distance method (method.dist) was “maximum”. The confidence intervals were overlaid on the heatmap using the ggplot2 package.

FIG. 7 shows increased sensitivities with antigen combinations. Columns filled with white represent specificity (%) and columns filled with black represent sensitivity (%) in NH, F+E−, and F+E+ groups. Columns filled with solid color indicate individual protein and columns filled with texture indicate combined proteins.

FIGS. 8A-8C show reactivity of MAP orthologs on ELISA. FIG. 8A is a group comparison between NL and F+E+ in selected MAP recombinant protein ELISA. FIG. 8B shows correlation between MAP ELISA and MTB protein array. FIG. 8C shows sensitivity and specificity with individual MAP recombinant protein and combined 4 proteins at M+2SD cutoff.

FIG. 9 shows distribution of serum multiplex assay median fluorescent intensity (MFI) to each antigen among groups. The violin plots show the distribution shape of the data among NL (n=60), F+E− (n=60), and F+E+ (n=60). The box plots in the center represent the interquartile range. The vertical line on each box represents 1.5× interquartile range (IQR), and the dots represent outliers. The symbol * indicates p<0.05 when MFI in infected groups (F+E− or F+E+) compared to MFI in NL group, and ** indicates p<0.01 based on Mann-Whitney's U test.

FIG. 10 shows distribution of milk multiplex assay MFI to each antigen among groups. The violin plots show the distribution shape of the data among the NL (n=30), F+E− (n=30), and F+E+ (n=30) groups. The box plots in the center represent the interquartile range. The vertical line on each box represents 1.5× interquartile range (IQR), and the dots represent outliers. The symbol ** indicates p<0.01 based on Mann-Whitney's U test when MFI in infected groups (F+E− or F+E+) compared to MFI in NL group.

FIGS. 11A-11F show ROC curves depicting reactivity for each antigen with both serum and milk samples. FIGS. 11A-11C show serum (n=180, 60 each group), and FIGS. 11D-11F show milk (n=90, 30 each group). Group All represents 180 samples in serum and 90 in milk; F+E+/NL includes group NL and F+E+ (n=120 in serum; n=90 in milk); F+E−/NL includes group NL and F+E+ (n=120 in serum; n=90 in milk).

FIG. 12 shows a comparison of serum antibody reactivity of multiplex and ELISA to recombinant proteins. ROC curves of serum multiplex reactivity to 6 recombinant MAP proteins were compared with those of serum ELISA using the same recombinant antigens (NL n=30, F+E+ n=60). The ROC curves represent data from serum ELISA or from multiplex assay as indicated. The tables inside the plots describe the name of antigen, sensitivity, specificity and AUC.

FIGS. 13A-13B show a comparison of milk multiplex and ELISA antibody reactivity. Milk multiplex antibody reactivity to recombinant MAP proteins was compared with IDEXX ELISA test results in the F+E− (n=30) and NL (n=30). FIG. 13A is a Table of AUC, cutoff, Sensitivity and Specificity; FIG. 13B shows ROC curves.

FIGS. 14A-14B show heat maps of forty MAP1569 peptide arrays exposed to 20 negative (FIG. 14A) and 20 positive (FIG. 14B) cattle sera. Each dot represents a 15-amino acid peptide within the full-length MAPI 569 protein. The brighter the dot, the more intense the reaction to that peptide occurred with the indicated serum sample.

### DETAILED DESCRIPTION OF THE INVENTION

The following definitions and introductory matters are provided to facilitate an understanding of the present invention.

Johne's disease is a serious disease caused by infection with MAP. The bacteria can lie dormant in animals for many years before symptoms appear but can be easily transmitted between animals in a herd.

The present inventors have found that improved detection of Johne's disease can be achieved with several novel antigens that may be detected by the methods of the invention. Because the kit and method of the present invention provide a result in a quick and relatively inexpensive manner, they may be used in a method of general health screening.

The present invention may be used to screen large populations to determine the levels of antibody response and therefore exposure to MAP. This may include screening for latent MAP infection.

It is common for certain populations to include high numbers of individuals who are carriers of latent MAP. These are individuals who are infected with the bacteria but do not have any active disease. However, in populations in which infection with latent MAP is high, there is an increase in the incidence of MAP. Identifying populations or groups of individuals who are infected with latent MAP can help predict where outbreaks of disease are likely.

These findings have provided the means for producing novel diagnostics for the detection of MAP infection in a subject, and novel prognostic indicators for the progression of infection or a disease state associated therewith, such as Johne's disease. Preferably, the antigen sequences and/or proteins are useful for the early diagnosis of infection or disease. It will also be apparent to the skilled person that such prognostic indicators as described herein may be used in conjunction with therapeutic treatments for MAP or an infection associated therewith.

Accordingly, the present invention provides the means for producing novel diagnostics for the detection of MAP infection in a subject, and novel prognostic indicators for the progression of infection or a disease state associated therewith, either by detecting the sequences of the invention or as part of a multi-analyte test. Preferably, the antigen proteins are useful for the early diagnosis of infection or disease. It will also be apparent to the skilled person that such prognostic indicators as described herein may be used in conjunction with therapeutic treatments for MAP or an infection associated therewith.

It will be apparent from the disclosure that a preferred antigen peptide, fragment or epitope comprises an amino acid sequence of at least about 5 consecutive amino acid residues as disclosed in the sequences herein. This includes any peptides comprising an N-terminal extension of up to about 5 amino acid residues in length and/or a C-terminal extension of up to about 5 amino acid residues in length.

It is within the scope of the present invention for the isolated or recombinant antigen protein of MAP to comprise one or more labels or detectable moieties e.g., to facilitate detection or isolation or immobilization. Preferred labels include, for example, biotin, glutathione-S-transferase (GST), FLAG epitope, hexa-histidine, β-galactosidase, horseradish peroxidase, streptavidin or gold.

The present invention also provides a fusion protein comprising one or more antigen peptides, fragments or epitopes according to any embodiment described herein. For example, the N-terminal and C-terminal portions can be fused via an internal cysteine residue. The skilled artisan will be aware that such an internal linking residue is optional or preferred and not essential to the production, or every use, of a fusion protein. However, preferred fusion proteins may comprise a linker separating an antigen peptide from one or more other peptide moieties, such as, for example, a single amino acid residue (e.g., glycine, cysteine, lysine), a peptide linker (e.g., a non-immunogenic peptide such as a poly-lysine or poly-glycine), poly-carbon linker comprising up to about 6 or 8 or 10 or 12 carbon residues, or a chemical linker. Such linkers may facilitate antibody production e.g., by permitting linkage to a lipid or hapten, or to permit cross-linking or binding to a ligand. The expression of proteins as fusions may also enhance their solubility.

Preferred fusion proteins will comprise the antigen protein, peptide, fragment or epitope fused to a carrier protein, detectable label or reporter molecule e.g., glutathione-S-transferase (GST), FLAG epitope, hexa-histidine, Pgalactosidase, thioredoxin (TRX) (La Vallie et al., Bio/Technology 11, 187-193, 1993), maltose binding protein (MBP), Escherichia coli NusA protein (Fayard, E. M. S., Thesis, University of Oklahoma, USA, 1999; Harrison, inNovations 11, 4-7, 2000), E. coli BFR (Harrison, inNovations 11, 4-7, 2000) and E. coli GrpE (Harrison, inNovations 11, 4-7, 2000).

The present invention also provides an isolated protein aggregate comprising one or more antigen peptides, fragments or epitopes according to any embodiment described herein. Preferred protein aggregates will comprise the protein, peptide, fragment or epitope complexed to an immunoglobulin e.g., IgA, IgM or IgG, such as, for example as a circulating immune complex (CIC). Exemplary protein aggregates may be derived, for example, from an antibody-containing biological sample of a subject.

The present invention also encompasses the use of the isolated or recombinant antigen protein of MAP or epitope thereof according to any embodiment described herein for detecting a past or present infection or latent infection by MAP in a subject, wherein said infection is determined by the binding of antibodies in a sample obtained from the subject to said isolated or recombinant protein or a fragment or epitope.

The present invention also encompasses the use of the isolated or recombinant antigen proteins of MAP for eliciting the production of antibodies that bind to MAP.

The present invention also provides an isolated nucleic acid encoding the isolated or recombinant antigen protein of MAP fragment or epitope thereof according to any embodiment described herein e.g., for expressing the immunogenic polypeptide, protein, peptide, fragment or epitope.

The present invention also provides a cell expressing the isolated or recombinant antigen protein of MAP or a fragment or epitope thereof according to any embodiment described herein. The cell may preferably consist of an antigen-presenting cell (APC) that expresses the antigen on its surface.

The present invention also provides an isolated or recombinant antibody or immune reactive fragment of an antibody that binds specifically to the isolated or recombinant antigen protein of MAP or fragment or epitope thereof according to any embodiment described herein, or to a fusion protein or protein aggregate comprising said antigen protein, peptide, fragment or epitope. Preferred antibodies include, for example, a monoclonal or polyclonal antibody preparation. This extends to any isolated antibody-producing cell or antibody-producing cell population, e.g., a hybridoma or plasmacytoma producing antibodies that bind to an antigen protein or immunogenic fragment of a peptide comprising a sequence derived from the sequence of an antigen protein disclosed herein.

The present invention also provides for the use of the isolated or recombinant antibody according to any embodiment described herein or an immune-reactive fragment thereof in medicine.

The present invention also provides for the use of the isolated or recombinant antibody according to any embodiment described herein or an immune-reactive fragment thereof for detecting a past or present (i.e., active) infection or a latent infection by MAP in a subject, wherein said infection is determined by the binding of the antibody or fragment to MAP antigen protein or an immunogenic fragment or epitope thereof present in a biological sample obtained from the subject.

The present invention also provides for the use of the isolated or recombinant antibody according to any embodiment described herein or an immune-reactive fragment thereof for identifying the bacterium MAP or cells infected by MAP or for sorting or counting of said bacterium or said cells.

The isolated or recombinant antibodies, or immune-reactive fragments thereof, are also useful in therapeutic, diagnostic and research applications for detecting a past or present infection, or a latent infection, by MAP as determined by the binding of the antibody to a MAP antigen protein or an immunogenic fragment or epitope thereof present in a biological sample from a subject (i.e., an antigen-based immunoassay).

Other applications of the subject antibodies include the purification and study of the diagnostic/prognostic antigen protein, identification of cells infected with MAP, or for sorting or counting of such cells.

The antibodies and fragments thereof are also useful in therapy, including prophylaxis, diagnosis, or prognosis, and the use of such antibodies or fragments for the manufacture of a medicament for use in treatment of infection by MAP. The present invention also provides a composition comprising the isolated or recombinant antibody according to any embodiment described herein and a pharmaceutically acceptable carrier, diluent or excipient.

The present invention also provides a method of diagnosing Johne's disease or an infection by MAP in a subject comprising detecting in a biological sample from said subject antibodies against antigen protein or fragment or epitope thereof, the presence of said antibodies in the sample is indicative of infection. In a related embodiment, the presence of said antibodies in the sample is indicative of infection. The infection may be a past or active infection, or a latent infection, however this assay format is particularly useful for detecting active infection and/or recent infection.

For example, the method may be an immunoassay, e.g., comprising contacting a biological sample derived from the subject with the isolated or recombinant antigen protein of MAP or fragment or epitope thereof according to any embodiment described herein for a time and under conditions sufficient for an antigen-antibody complex to form and then detecting the formation of an antigen-antibody complex. The sample is an antibody-containing sample e.g., a sample that comprises blood or serum or an immunoglobulin fraction obtained from the subject. The sample may contain circulating antibodies in the form of complexes antigenic fragments.

It is within the scope of the present invention to include a multi-analyte test in this assay format, wherein multiple antigenic epitopes are used to confirm a diagnosis obtained using an antigen peptide of the invention. In some embodiments four, five, or six antigens are used. The assays may also be performed in the same reaction vessel, provided that different detection systems are used to detect the different antibodies, e.g., labelled using different reporter molecules such as different colored dyes, fluorophores, radionucleotides or enzymes.

The present invention also provides a method of diagnosing Johne's disease or infection by MAP in a subject comprising detecting in a biological sample from said subject an antigen protein or a fragment or epitope thereof, wherein the presence of said protein or immunogenic fragment or epitope in the sample is indicative of disease, disease progression or infection. In a related embodiment, the presence of said protein or immunogenic fragment or epitope in the sample is indicative of infection. For example, the method can comprise an immunoassay e.g., contacting a biological sample derived from the subject with one or more antibodies capable of binding to a protein or an immunogenic fragment or epitope thereof, and detecting the formation of an antigen-antibody complex. In a particularly preferred embodiment, an antibody is an isolated or recombinant antibody or immune reactive fragment of an antibody that binds specifically to the isolated or recombinant protein of MAP or a fragment or epitope thereof according to any embodiment described herein or to a fusion protein or protein aggregate comprising said immunogenic antigen protein, peptide, fragment or epitope.

The present invention also provides a method for determining the response of a subject having Johne's disease or an infection by MAP to treatment with a therapeutic compound for said Johne's disease or infection, said method comprising detecting an antigen protein or an immunogenic fragment or epitope thereof in a biological sample from said subject, wherein a level of the protein or fragment or epitope that is enhanced compared to the level of that protein or fragment or epitope detectable in a normal or healthy subject indicates that the subject is not responding to said treatment or has not been rendered free of disease or infection.

The present invention also provides a method of monitoring disease progression, responsiveness to therapy or infection status by MAP in a subject comprising determining the level of an antigen protein or an immunogenic fragment or epitope thereof in a biological sample from said subject at different times, wherein a change in the level of the protein, fragment or epitope indicates a change in disease progression, responsiveness to therapy or infection status of the subject. In a preferred embodiment, the method further comprises administering a compound for the treatment of Johne's disease or infection by MAP when the level of protein, fragment or epitope increases over time.

The present invention also provides a method of treatment of Johne's disease or infection by MAP comprising: (i) performing a diagnostic method according to any embodiment described herein thereby detecting the presence of MAP infection in a biological sample from a subject; and (ii) administering a therapeutically effective amount of a pharmaceutical composition to reduce the number of MAP bacteria in the intestinal system of the subject.

The present invention also provides a method of treatment of Johne's disease in a subject comprising performing a diagnostic method or prognostic method as described herein. In one embodiment, the present invention provides a method of prophylaxis comprising: (i) detecting the presence of MAP infection in a biological sample from a subject; and (ii) administering a therapeutically effective amount of a pharmaceutical composition to reduce the number of MAP bacteria in the intestinal system of the subject.

Accordingly, this invention also provides an immunogenic antigen protein or one or more immunogenic peptides or immunogenic antigen fragments or epitopes thereof in combination with a pharmaceutically acceptable diluent. Preferably, the protein or peptide(s) or fragment(s) or epitope(s) thereof is(are) formulated with a suitable adjuvant.

The present invention also provides a kit for detecting MAP infection in a biological sample, said kit comprising: (i) one or more isolated antibodies or immune reactive fragments thereof that bind specifically to the isolated or recombinant antigen protein of MAP or an immunogenic peptide or immunogenic fragment or epitope thereof according to any embodiment described herein or to a fusion protein or protein aggregate comprising said immunogenic protein, peptide, fragment or epitope; and (ii) means for detecting the formation of an antigen-antibody complex, optionally packaged with instructions for use.

The assays described herein are amenable to any assay format. Such methods are well known in the art and include but are not limited to solid phase ELISA, immunoprecipitation, immunofluorescence, Western blot, dot blot, radioimmunoassay, flow cytometry (FACS analysis), immunocytochemistry, multiplex bead-based immunoassays, flow through immunoassay formats, capillary formats, and for the purification or isolation of immunogenic proteins, peptides, fragments and epitopes and CICs.

Enzyme-linked immunosorbent assays (ELISA) are standard in the art and can be found at, for example, Ausubel, F. M. et al., (Current Protocols in Molecular Biology, Volume 2, pp. 11.2.1-11.2.22, John Wiley & Sons, Inc., 1991). ELISA typically uses an enzymatic reaction to convert substrates into products having a detectable signal (e.g., fluorescence). Each enzyme in the conjugate can covert hundreds of substrates into products, thereby amplifying the detectable signal and enhancing the sensitivity of the assay. ELISA assays are understood to include derivative and related methods, such as sandwich ELISA and microfluidic ELISA.

Accordingly, the present invention also provides a solid matrix having adsorbed thereto an isolated or recombinant antigen protein or an immunogenic antigen peptide or immunogenic antigen fragment or epitope thereof according to any one embodiment described herein or a fusion protein or protein aggregate comprising said immunogenic protein, peptide, fragment or epitope. For example, the solid matrix may comprise a membrane, e.g., nylon or nitrocellulose. Alternatively, the solid matrix may comprise a polystyrene or polycarbonate microwell plate or part thereof (e.g., one or more wells of a microtiter plate), a dipstick, a glass support, or a chromatography resin.

In an alternative embodiment, the invention also provides a solid matrix having adsorbed thereto an antibody that binds to an isolated or recombinant protein or an immunogenic peptide or immunogenic fragment or epitope thereof according to any embodiment described herein or to a fusion protein or protein aggregate comprising said immunogenic protein, peptide, fragment or epitope. For example, the solid matrix may comprise a membrane, e.g., nylon or nitrocellulose. Alternatively, the solid matrix may comprise a polystyrene or polycarbonate microwell plate or part thereof (e.g., one or more wells of a microtiter plate), a dipstick, a glass support, or a chromatography resin.

It is clearly within the scope of the present invention for such solid matrices to comprise additional antigens and/or antibodies as required to perform an assay described herein, especially for multianalyte tests employing multiple antigens or multiple antibodies.

In a multiplexed assay, multiple analytes are simultaneously measured. Each polypeptide antigen is positioned such that it is individually addressable. For example, the polypeptide antigens can be immobilized in a substrate. The multiplex bead-based immunoassays used to practice the present invention include but are not limited to the Luminex xMAP technology described in U.S. Pat. Nos. 6,599,331, 6,592,822, and 6,268,222, all of which are herein incorporated by reference in their entirety. The Luminex system, which utilizes fluorescently labeled microspheres, allows up to 100 analytes to be simultaneously measured in a single microplate well, using very small sample volumes. For example, a recombinant MAP antigen can be coupled to a bead with one distinct internal dye and is then recognized by a MAP antigen-specific antibody in a sample. This specific antibody is bound by a secondary antibody that is attached to a fluorescent reporter dye. Within the Luminex analyzer, lasers excite the internal dyes that identify the distinct bead color corresponding to one MAP antigen, and the reporter dye identifying the amount of MAP-specific antibodies captured during the assay. Multiple beads with different MAP antigens and different bead color codes can be combined in one assay run. Multiple readings are made on each bead set and result in an individual fluorescent signal for each bead assay. In this way, the technology allows rapid and accurate analysis of up to 100 unique assays within a single sample. However, other multiplex platforms can also be used, and the invention is not intended to be limited by the type of multiplex platform selected.

As used herein, “a,” “an” or “the” can mean one or more than one. For example, “a” cell can mean a single cell or a multiplicity of cells.

As used herein, “and/or” refers to and encompasses any and all possible combinations of one or more of the associated listed items, as well as the lack of combinations when interpreted in the alternative (“or”).

The term “isolated” as used herein means the protein or polypeptide or immunologically reactive fragment or nucleic acid of this invention is sufficiently free of contaminants or cell components with which polypeptides and/or nucleic acids normally occur. “Isolated” does not mean that the preparation is technically pure (homogeneous), but it is sufficiently pure to provide the polypeptide or nucleic acid in a form in which it can be used in methods of this invention.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The term “epitope” means an antigenic determinant that is specifically bound by an antibody. Epitopes usually consist of surface groupings of molecules such as amino acids and/or sugar side chains and usually have specific three-dimensional structural characteristics, as well as specific charge characteristics. As used herein, “epitope” refers to at least about 3 to about 5, or about 5 to about 10 or about 5 to about 15, and not more than about 1,000 amino acids (or any integer therebetween) (e.g., 5-12 amino acids or 3-10 amino acids or 4-8 amino acids or 6-15 amino acids, etc.), which define a sequence that by itself or as part of a larger sequence, binds to an antibody generated in response to such sequence or stimulates a cellular immune response. There is no critical upper limit to the length of the fragment, which can comprise the full-length of the protein sequence, nearly the full-length of the protein sequence, or even a fusion protein comprising two or more epitopes from a single or multiple MAP proteins.

An “immunologically reactive fragment,” “immunogenic fragment” or “antigenic fragment” of a protein refers to a portion of the protein or peptide that is immunologically reactive with a binding partner, e.g., an antibody, which is immunologically reactive with the protein or peptide itself. In some embodiments, an “immunogenic fragment” of this invention can comprise one, two, three, four or more epitopes of a protein of this invention.

In some embodiments, the terms “immunologically reactive fragment,” “immunogenic fragment” or “antigenic fragment” are used to describe a fragment or portion of a protein or peptide that can stimulate a humoral and/or cellular immune response in a subject. An immunologically reactive fragment, immunogenic fragment or antigenic fragment of this invention can comprise, consist essentially of and/or consist of one, two, three, four or more epitopes of one or more MAP proteins of this invention.

An immunologically reactive fragment, immunogenic fragment or antigenic fragment can be any fragment of contiguous amino acids of a MAP protein of this invention, including but not limited to MAP1272c, MAP1569, MAP2121c, MAP2942c, MAP2609, MAP1201c+2942c, MAP1201c, 2942c, MAP0019c, MAP0117, MAP0123, MAP0357, MAP0433c, MAP0616c, MAP0646c, MAP0858, MAP0953, MAP1152, MAP1224c, MAP1298, MAP1506, MAP1525, MAP1561c, MAP1651c, MAP1761c, MAP1782c, MAP1960, MAP1968c, MAP1986, MAP2093c, MAP2100, MAP2117c, MAP2158, MAP2187c, MAP2195, MAP2288c, MAP2447c, MAP2497c, MAP2694, MAP2875, MAP3039c, MAP3305c, MAP3527, MAP3531c, MAP3540c, MAP3762c, MAP3773c, MAP3852c, MAP4074, MAP4143, MAP4225c, MAP4231, MAP4339, and combinations thereof, the amino acid sequences of each of which are provided herein and can be for example, 5, 10, 15, 20, 25, 30, 35, 40, 45, 50, 75, 100, 150, 200, 250, 300, 350, 400, 450, 500, 550, 600, 650, 700, 750, 800, 850, 900, 950 or 1000 amino acids in length, dependent upon the total number of amino acids of the full length protein.

A fragment of a polypeptide or protein of this invention can be produced by methods well known and routine in the art. Fragments of this invention can be produced, for example, by enzymatic or other cleavage of naturally occurring peptides or polypeptides or by synthetic protocols that are well known. Such fragments can be tested for one or more of the biological activities of this invention according to the methods described herein, which are routine methods for testing activities of polypeptides, and/or according to any art-known and routine methods for identifying such activities. For example, to identify immunogenic fragments derived from the MAP proteins, peptides synthesized in a peptide array are prepared and screened with sera. Such production and testing to identify biologically active fragments and/or immunologically reactive fragments of the polypeptides described herein would be well within the scope of one of ordinary skill in the art and would be routine.

The term “sample” describes any type of sample suspected to contain a desired target protein to be assayed for detection of such target protein. In some embodiments a biological sample from a subject suspected of infected with MAP will be used, such as blood, plasma, serum, or milk, or other bodily fluids that may contain the biomarker. These may include, for example, plasma, serum, spinal fluid, lymph fluid, secretions from the respiratory, gastrointestinal, or genitourinary systems including tears, saliva, milk, urine, semen, hepatocytes, and red or white blood cells or platelets. In some cases, a tissue sample may be used in the assay or processed for use in the assay, for example, by a conventional method used to extract proteins from the sample.

“Mammals” include any warm-blooded vertebrates of the Mammalia class, including humans. As used herein, the term “ruminant” means an even-toed, hoofed animal that has a complex 3- or 4-chamber stomach and that typically re-chews what the ruminant has previously swallowed. Some non-exhaustive examples of ruminants include cattle, sheep, goats, oxen, musk, ox, llamas, alpacas, guanicos, deer, bison, antelopes, camels, and giraffes.

As used herein, the term “infection” shall be understood to mean invasion and/or colonization by a microorganism and/or multiplication of a micro-organism, in particular, a bacterium or a virus, in the intestinal tract of a subject. Such an infection may be unapparent or result in local cellular injury. The infection may be localized, subclinical and temporary or alternatively may spread by extension to become an acute or chronic clinical infection. The infection may also be a past infection wherein residual antigen, or alternatively, reactive host antibodies that bind to isolated antigen protein or peptides, remain in the host. The infection may also be a latent infection, in which the microorganism is present in a subject, however the subject does not exhibit symptoms of disease associated with the organism.

The present invention is further illustrated by the following examples, which should not be considered as limiting in any way.

### EXAMPLES

### Example 1: Identification of Sero-Reactive Antigens for the Early Diagnosis of Johne's Disease in Cattle

Johne's disease (JD) is a chronic granulomatous intestinal inflammatory disease that results from infection with Mycobacterium avium subspecies paratuberculosis (MAP) [1]. JD results in more than $200 million in annual losses to the US dairy industry each year [2]. Despite considerable control efforts, JD remains a major problem for producers and the industry due to high prevalence rates (68% of all US dairy herds and 95% of those with over 500 cows have at least one JD positive animal) [3]. Although animals are infected early in life through ingestion of bacilli via the fecal-oral route or from colostrum, JD takes several years to manifest [4, 5]. During this extremely long sub-clinical phase, infected animals are continuously or intermittently shedding the pathogen into the environment and spreading the disease. However, it is very difficult to reliably identify infected from non-infected animals during early infection, especially in animals that are intermittently shedding. Hence, the development of highly sensitive and specific diagnostics has the potential to be transformative in the field and is key for control of JD and enhancement of animal health.

Due to low sensitivity of current serological assays (particularly ELISAs) which use relatively crude cellular extracts, several studies focused on identification of individual antigens soon after the complete genome sequence of MAP was published [6]. These include studies that used bioinformatics' screens to predict function and localization of proteins, followed by proteomic analyses of cell wall associated proteins [7], MAP culture filtrates [8]; surface proteins expressed in macrophage [9]; proteins that respond to stress during in vitro culture [10]; proteomic comparison of MAP with Mycobacterium aviam subspecies avium [11]; as well as a dot-blot based protein arrays of recombinant proteins representing secreted or cell wall associated proteins [12] to identify MAP antigens of potential diagnostic utility with varying degrees of success. For instance, studies have shown that sera from experimentally infected cattle recognized specific MAP proteins at a very early stage of the infection, or with either mild or paucibacillary infections that were presumably from subclinical animals and well before antibodies were detected by using commercial ELISA assays [13-15], suggesting that a subset of MAP proteins may be seroreactive during early (subclinical) infection. However, none of these candidates have proved of clinical utility or have shown potential to replace the extant whole-cell antigen based commercially available ELISAs.

To date, more than 200 recombinant proteins have been tested for antigenicity and more than 800 recombinant proteins have been overexpressed for antigen discovery [12-16]. However, this represents only approximately 20% of predicted proteins in the MAP proteome (n=4,350) [6]. Given the significant time and financial costs associated with cloning, expressing and purifying additional proteins from MAP, we have recently explored the possibility of leveraging the commercially available whole proteome microarray from Mycobacterium tuberculosis (MTB), a closely related pathogen [17]. The MTB proteome array contains ˜4,000 features (3,864 unique MTB genes) covering 97% of the genome and has previously been successfully used to identify biomarkers of active TB infection from a global collection of human and non-human primate serum and plasma samples [18, 19]. Our preliminary pairwise comparison of amino acid sequence between orthologous proteins in MAP and MTB showed an average of 62% identity (range 19% to 100%) with more than half sharing >75% identity [17]. Further bioinformatic analyses confirmed that the MTB proteome array contains ˜800 MAP orthologs that have previously been expressed and an additional ˜1,900 having significant levels of homology with their MAP orthologs that have not been expressed.

Our pilot studies conducted using serum samples from 9 MAP-infected cows (6 clinical and 3 subclinical) and 3 uninfected control and MTB full-proteome chips revealed more than 700 MTB reactive antigens [17], less than 200 of which represent orthologs that were already represented among the expressed MAP proteins. Probing the MTB array with serum from MAP-infected animals resulted in the identification of more than 500 antigens, for which several of these proteins displayed greater reactivity with serum from subclinical animals as compared to clinical stage animals. This suggests that the MTB protein array has considerable potential to identify a significant number of new candidate antigens detectable during early stages of disease. However, only a very small number of serum samples were used in this preliminary screen, and hence these results needed to be corroborated with an expanded set of well-characterized samples, and further validated for use in immunoassays. We here report immune profiling using a large collection of well-characterized serum samples from MAP-infected cows and negative controls with the MTB protein microarray, as well as the development of specific and sensitive ELISA assays using defined MAP antigens.

### Materials and Methods

### Bovine Serum Samples

All serum samples were collected as part of the Johne's Disease Integrated Program (JDIP, mycobacterialdiseases.org) diagnostic standards sample collection project. In brief, the 180 samples used in these studies were collected from cows housed in 13 dairy farms from 4 states: California, Georgia, Minnesota, and Pennsylvania. The herd size ranged from 66 to 1,400 and prevalence of JD ranged from 0 to 53.30% based on serum ELISA tests conducted prior to sample collection. All herds were negative for bovine TB. As JDIP diagnostic standards sample collection study designed, each cow was tested for level of MAP shedding in feces as well as serological reactivity. MAP shedding was determined by fecal culture using Herrold's solid medium (HEYM) and two different liquid culture medium systems, BACTEC MGIT and Trek (Becton, Dickinson and Company, Franklin Lakes, N.J.); all fecal cultures were confirmed by acid fast staining and PCR. Fecal qPCR was performed for each animal with the LT TaqMan (ThermoFisher, Waltham, Mass.) and Tetracore (Tetracore, Rockville, Md.) assays. Serum and milk ELISA tests were performed using both IDEXX kit (IDEXX Laboratories, Inc., ME) and ParaChek (ThermoFisher, Waltham, Mass.) according to the manufacturers' instructions. Based on the result of fecal and serological tests, cows were stratified into three groups: both fecal and serological tests negative (n=60), fecal test positive and serological test negative (F+E−, i=60) and both fecal and serological tests positive (F+E+, n=60). Based on the previously observed prevalence of JD in each originating farm (according to serological tests conducted one year before above samples collected), cows in the negative group were further stratified into two groups: negative from low-exposure herds (NL, n=30) if they were from farms that had no recent evidence of JD prevalence (0%) and negative from high-exposure herds (NH, n=30) if the farm had evidence of previous JD prevalence (0.60 to 53.30%).

All serum samples were collected as part of the Johne's Disease Integrated Program (JDIP, mycobacterialdiseases.org) diagnostic standards sample collection project number 2008-55620-18710. Animal use protocols were approved by the Pennsylvania State University IACUC numbers 34625 and 43309.

### Microarray Fabrication and Probing

The MTB microarray fabrication and probing were conducted in Antigen Discovery Inc. (ADI, Irvine, Calif.) as described previously [18, 19]. The microarrays carried 3,963 MTB protein spots, which corresponded to more than 97% of the ORFs in the MTB H37Rv genome [18]. Briefly, using genomic DNA as a template, all open reading frames in the MTB H₃₇Rv genome were amplified using custom PCR primers. Genes >3 kb in length were amplified as overlapping fragments. PCR products were cloned into a linearized T7 vector using in vivo recombination cloning. Using individually purified plasmids, MTB proteins were expressed in an E. coli-based in vitro transcription and translation system (IVTT) (5 Prime, Gaithersburg, Md.). The resulting IVTT reactions were printed as single spots without further purification into custom 3-pad nitrocellulose-coated Oncyte Avid slides (Grace Bio-Labs, Bend, Oreg.) using an Omni Grid 100 microarray printer (Digilabs, Inc., Marlborough, Mass.) in 4×4 sub-array format, with each subarray comprising 18×18 spots. Each sub-array included negative control spots carrying IVTT reactions without DNA templates, purified proteins spots of previously identified MTB biomarkers, as well as positive control spots for the hybridization. Quality control was carried out by probing a sample of chips from each print run using a monoclonal antibody against the N-terminal polyhistidine tag, the C-terminal HA tag and selected reference serum. Cryopreserved serum samples were thawed on ice and pre-incubated with E. coli lysate to absorb anti-E. coli and cross-reactive antibodies. Prior to incubation with serum, slides were re-hydrated and blocked for 30 minutes using Blocking Buffer (Main Manufacturing, Sanford, Me.). Serum samples were diluted 1:200 and incubated on arrays at 4° C. overnight with gentle agitation. Bound IgG antibodies were detected with a biotinylated anti-bovine IgG secondary antibody (Jackson ImmunoResearch, West Grove, Pa.), followed by incubation with Surelight-P3 fluorochrome conjugated to streptavidin (Columbia Biosciences, Columbia, N.Y.). Slides were then dried and scanned in a Genepix 4300A microarray scanner (Molecular Devices, San Diego, Calif.). The scanner laser power and PMT gain were calibrated daily to intensities obtained from reference sera to control for day-to-day variation. Fluorescence intensity values for each spot were quantified using GenePix Pro software, and data were exported in comma separated values (CSV) format (intensity data accessible via scholarsphere.psu.edu/concern/generic_works/hhm50ts37m).

### Data Analysis

The intensity data files in CSV format were read, processed and analyzed using an automated data analysis pipeline developed at ADI that was implemented in R (r-project.org). Spot intensity measurements were converted into a single data matrix of local background-subtracted intensities. The row names of the data matrix are unique spot identifiers that link to a spot annotation database, and the column names are unique sample identifiers that link to a sample information database. For each sample, quality checks were performed for possible missing spots, contaminations and unusual background variation. The data were also inspected for the presence of subtle systematic effects and biases (probing day, slide, pad, print order, etc). Once the data passed quality assurance, the final dataset utilized for analysis was obtained by the following steps: (1) log₂ transformation of raw intensities; (2) for each sample, calculation of the median of the IVTT negative control spots; and finally (3) subtraction of the sample-specific IVTT negative control medians. An antigen is classified as highly reactive to a given sample if its normalized intensity value is greater than 0.5 (the raw intensity is at least approximately 1.4× the sample's median IVTT negative control). An individual's antibody breadth scores are determined by its count of reactive antigens. Antibody breadth profiles were compared between groups using Poisson regression. Normalized data were modeled using parametric and non-parametric tests for between-group comparisons. For complex data sets, comparisons were made using multivariate linear regression or linear mixed models with random effects for longitudinal data. All p-values were adjusted for the false discovery rate as previously described [20].

### ELISA Assay for Selected MAP Recombinant Proteins

ELISA assays were conducted for selected MAP recombinant proteins (their MTB orthologs were identified as significantly reactive antigens) with serum samples from NL and F+E+ groups. The procedure was adapted from our previously described protocol [17] with a minor modification. ELISA 96-well microplates were coated with 50 μl/well of 1 μg/ml recombinant MAP protein or 0.5 μg/ml MBP/LacZ (fusion protein from cloning vector) in carbonate/bicarbonate buffer 0.1 M pH 9.6. Plates were sealed and incubated overnight at 4° C., then washed three times with 1×PBS, pH 7.4 containing 0.1% Tween 20 (PBS-T). Wells were blocked by adding 200 μl/well of PBS-T containing 1% bovine serum albumin (PBS-T-BSA) and incubated at room temperature for 1 hour before washing the plate three times with PBS-T. Serum samples diluted 1:250 in PBS-T-BSA were added to each well (100 μl/well) and incubated at room temperature for 1 hour before washing six times with PBS-T. Then 100 μl/well of anti-goat IgG peroxidase conjugate (Vector Labs, Buringame, Calif., USA) diluted 1:10,000 in PBS-T-BSA was added to all wells and incubated at room temperature for 1 hour before the plates were again washed six times with PBS-T. Finally, 100 μl/well of tetra methylbenzidine (TMB) SureBlue solution (KPL, Gaithersburg, Md., USA) was added and the reaction incubated for 10-15 minutes at room temperature with no light, before the reaction was stopped with 100 μl/well of 1.0 N HCl solution. The spectrophotometric reading of all wells was performed at 450 nm using a PowerWave XS2 microplate reader (BIoTek, Winooski, Vt., USA). The OD value of each sample was normalized by sample OD-MBP/LacZ OD to eliminate the non-specific background produced by anti-MBP/LacZ in each serum sample. The group t test was performed using GraphPad software (graphpad.com) and the significance of correlation of coefficient was determined using an online statistical computation tool (vassarstats.net).

### Logistic Regression Analysis

To determine which antigens had significantly different normalized intensities values among the 4 groups (NL, NH, F+E−, F+E+), ordinal logistic regression models were fitted, using PROC LOGISTIC in SAS (version 9.2, 2009: SAS Institute Inc., Cary, N.C.). Such models are appropriate for outcomes with more than two categories, as in this study, where the outcome was group with 4 categories (NL, NH, F+E−, F+E+). Each antigen was included in a model one at a time; all models also included lactation number of the cow, day-in-milk, and herd size. In each model, the generalized logit function was specified; each nonbaseline category is compared to the baseline category. In each model run, 180 observations were read in, but only 167 were used in the analysis, due to missing values for some covariates. Statistical significance was considered at alpha=0.05.

The output produced was in the form of odds ratios and their 95% confidence limits, for each category of group within a covariate (antigen, lactation number, day-in-milk, herd size). The baseline category varied with model, as it was desirable to have the baseline odds ratio value for each antigen be 1.0, and all comparisons made to that, within each antigen of interest, such that all comparison values were greater than 1.0. Therefore, each comparison (odds ratio for a particular group) gave the odds of belonging to a particular group compared to the odds of belonging to the baseline group. The odds ratio indicates how likely a certain antigen is associated with a particular group, compared to being associated with the baseline group. Another way to view the findings is thus: if, for a particular antigen, the odds ratio for NL is 1.0 (baseline group) and the odds ratio for F+E+ is 2.5, then for each unit increase in the normalized intensity value of the antigen, a cow is 2.5 times more likely to be classified as F+E+ than as NL.

### Results

### Identification of Highly Reactive Proteins

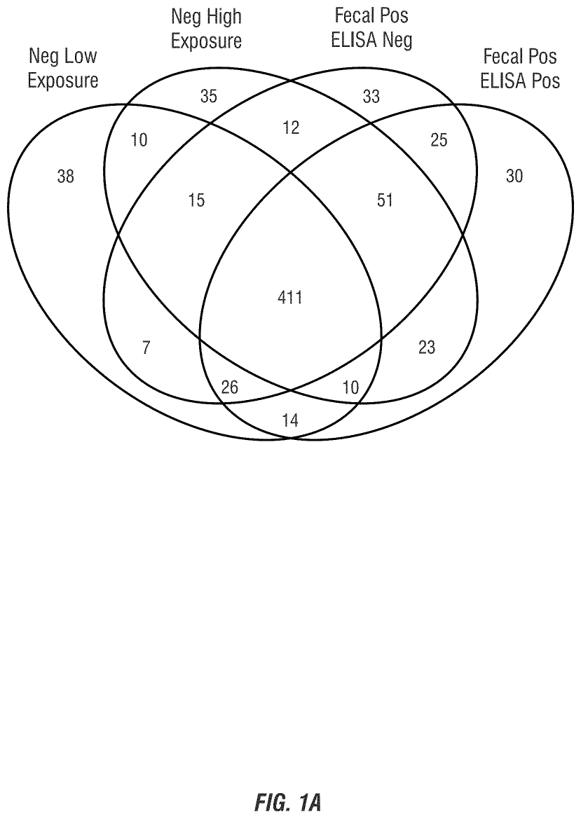

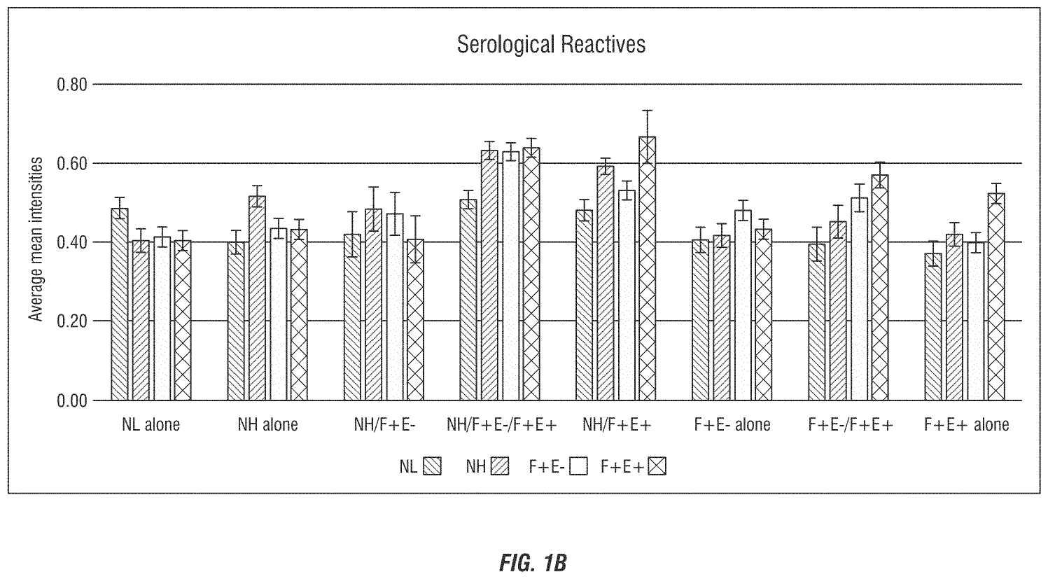

A total of 740 highly reactive antigens were identified based on normalized intensities at a 10% threshold with a distribution amongst the NL, NH, F+E−, and F+E+ groups as shown in the Venn diagram (FIG. 1A). In brief, the four ellipses show the total number of hits from the four groups of animals, with the majority of reactive proteins sharing cross-reactivity. If a highly reactive protein was identified in one group only, the protein was categorized as a unique protein. If a reactive protein was identified in two or more groups, the protein was categorized as a shared protein. Proteins were divided into 15 categories based on their unique or shared status among the groups. Unique proteins were identified in each of the 4 groups as: 38 in NL (5.1%), 35 in NH (4.7%), 33 in F+E− (4.5%) and 30 in F+E+ (4.1%) group respectively. There were a total of 411 proteins shared among all 4 groups, accounting for 55.5% of the total reactive proteins identified. The remaining proteins were shared within two (12.3%) or three (13.8%) of the groups. The average normalized intensities of proteins shared by all 4 groups were highest (>1.0), while the average intensities of the other groups were between 0.37 and 0.67 (FIG. 1B).

### Identification of Significantly Reactive Proteins

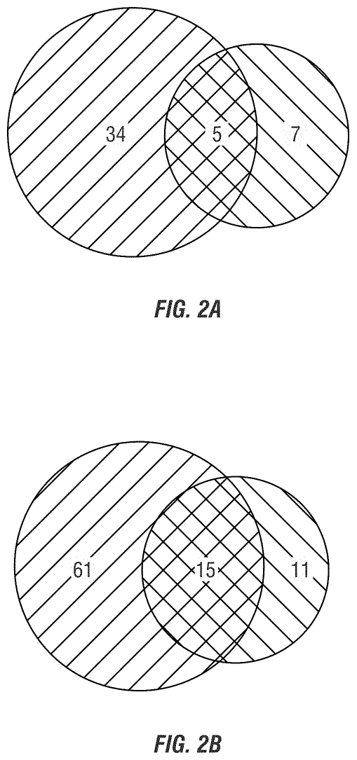

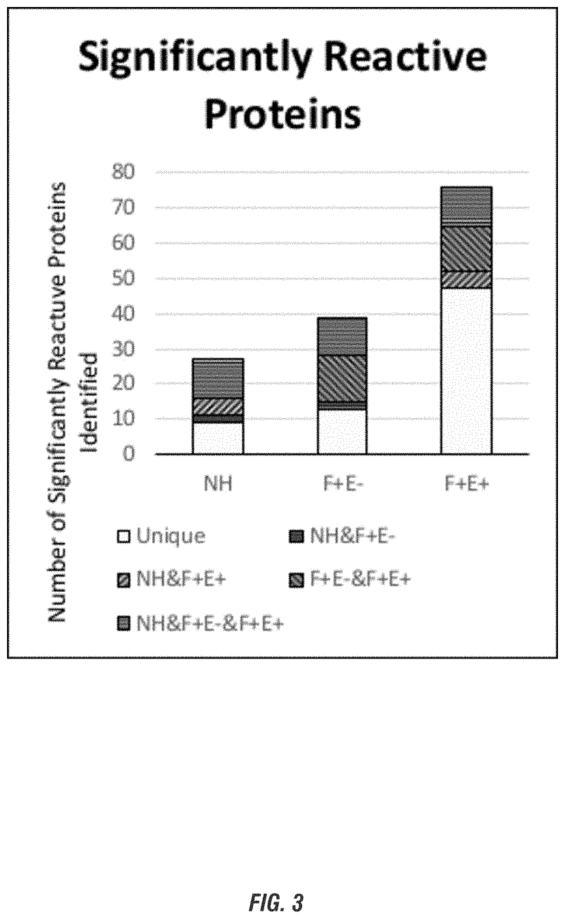

To determine which of the two groups of negative samples should be used as reference for group comparisons (NL or NH), we compared the mean intensities of the infected groups (F+E− and F+E+) with that of NL and NH individually as a reference. When mean intensities of the NL group were used as reference, 39 and 76 proteins were identified as significantly reactive proteins (P<0.05, based on group t test) in the F+E− and F+E+ groups, respectively. However, when the mean intensities of the NH group were used as reference, the number of significantly reactive proteins was reduced to 12 and 26 in the F+E− and F+E+ groups, respectively (FIG. 2). There were only 5 proteins shared in F+E− and 15 in F+E+ groups when mean intensities of NL and NH were used as a reference, respectively. In light of these observations, we chose to use NL alone as a reference for two reasons: 1) antigen identification was very reference-dependent and 2) samples from animals early in infection may contain antibodies recognizing MTB antigens in NH, and therefore candidate antigens may not be recognized if the mean intensities of NH are used as a reference. Mean normalized intensities in each group were compared to NL with a two-tailed t-test using ap-value <0.05 for significance. Of the 740 highly reactive proteins from the MTB array, approximately 130 were identified as significant (100 proteins) using this test. Among the 100 identified MTB proteins, there were a total of 69 unique proteins in the groups (9 in NH, 13 in F+E−, and 47 in F+E+) and 31 shared among groups (FIG. 3). On the other hand, if the mean intensities of proteins were significantly higher in the NL-alone group or groups shared with NL compared to the other three groups, these proteins were not considered as significant antigens, or “hits” (FIG. 1B). Significant antigens were identified in the following groups (number of significant antigens/total in group; percentage of significant antigens in the group): NH alone (5/35, 13.8%), NH/F+E− (1/12, 8.3%), NH/F+E+ (8/23, 34.8%), NH/F+E−/F+E+ (21/51, 41.2%), F+E− alone (6/33, 18.2%), F+E−/F+E+ (10/25, 40.0%) and F+E+ alone (11/30, 36.7%).

### Patterns of Intensity Changes Among Three Groups

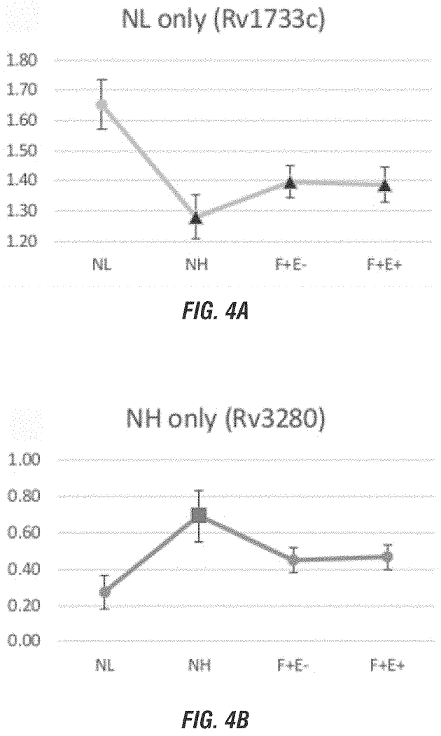

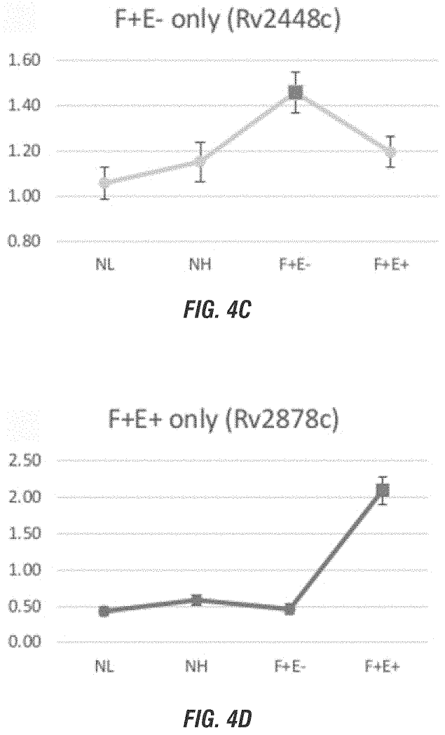

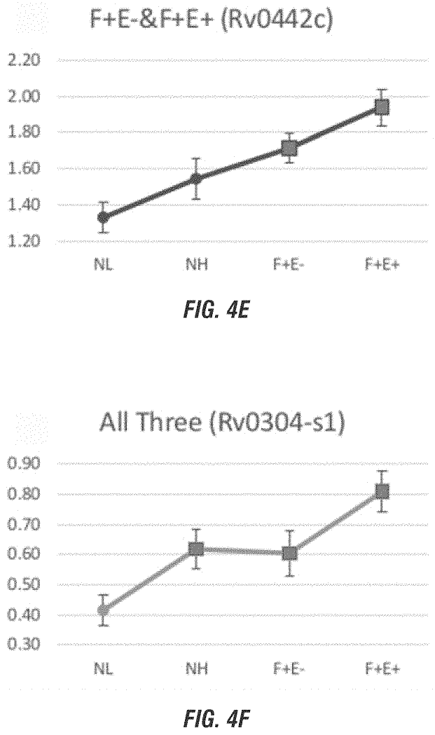

Compared to the normalized mean intensity of each protein in NL, there were 27 proteins with significantly higher and 15 with significantly lower intensities identified in the NH group (P<0.05). For the majority of proteins, the trend of intensity changes in the NH group was consistent with the changes in infected groups. For example, up to two thirds of proteins identified in NH were also found to have significantly higher (or lower) intensities in F+E− or F+E+ or both groups (FIG. 3) when compared with NL. Similar to NH, two thirds of the proteins identified in F+E− group were also shared with other groups, while in F+E+ group, more than 60% of proteins were unique. There were 6 patterns of intensity changes among three groups in comparison with NL (FIG. 4). The first pattern shows mean intensities are significantly higher only in NL. Among the 15 proteins with significantly lower intensities in NH, 14 were also found with lower intensities in F+E− and F+E+ groups. Only one protein, Rv0040c (ortholog MAP0047c), showed significant lower intensities in NH and F+E−, but significantly higher intensities in F+E+. Compared to intensities in NL, the proteins with lower intensities in infected groups were not considered reactive antigens, while proteins with significantly higher intensities in the other three groups were considered reactive antigens following the described 5 patterns. Proteins with significantly higher intensities only in NH group were considered to be antigens recognized only during the early stage of infection. Proteins with significantly higher intensities only in F+E− or only in F+E+ indicate that the antigen is recognized only in the middle or late stages of infection, while proteins with significantly higher intensities in both F+E− and F+E+ groups or in all three groups including NH, F+E− and F+E+, indicate antigens that can be recognized throughout the course of infection.

### Orthologs in MAP

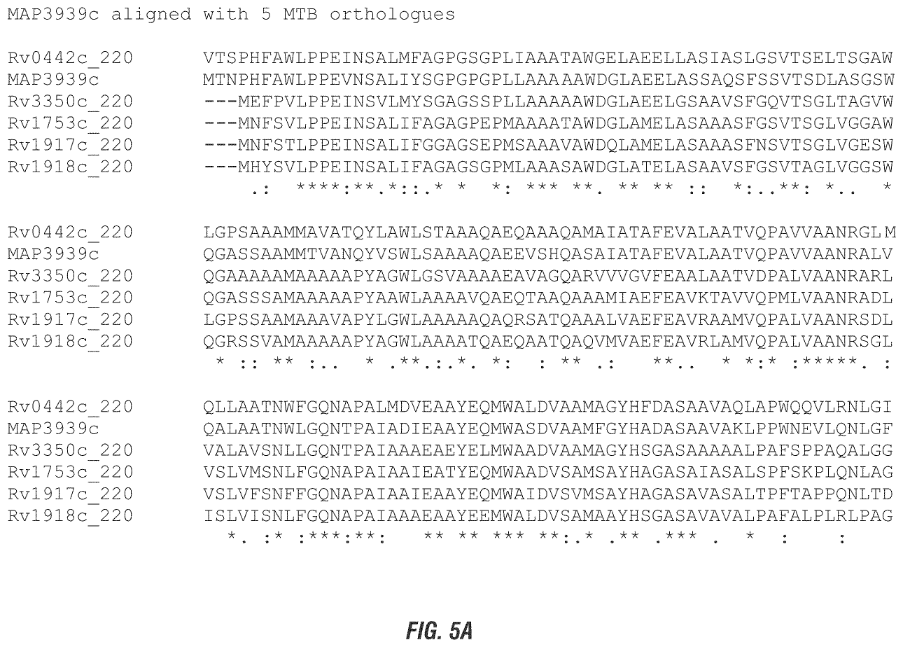

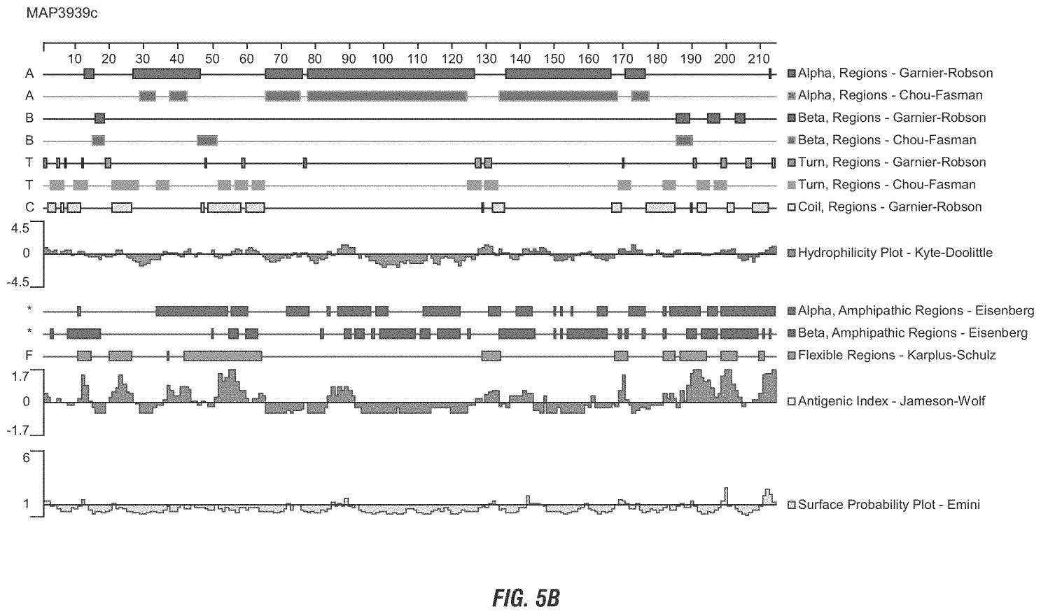

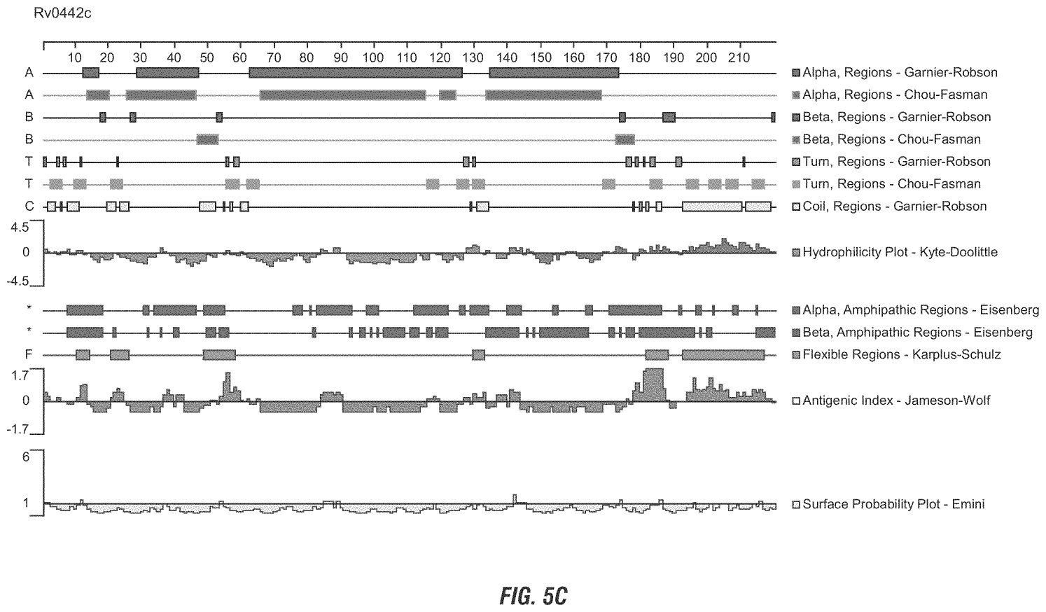

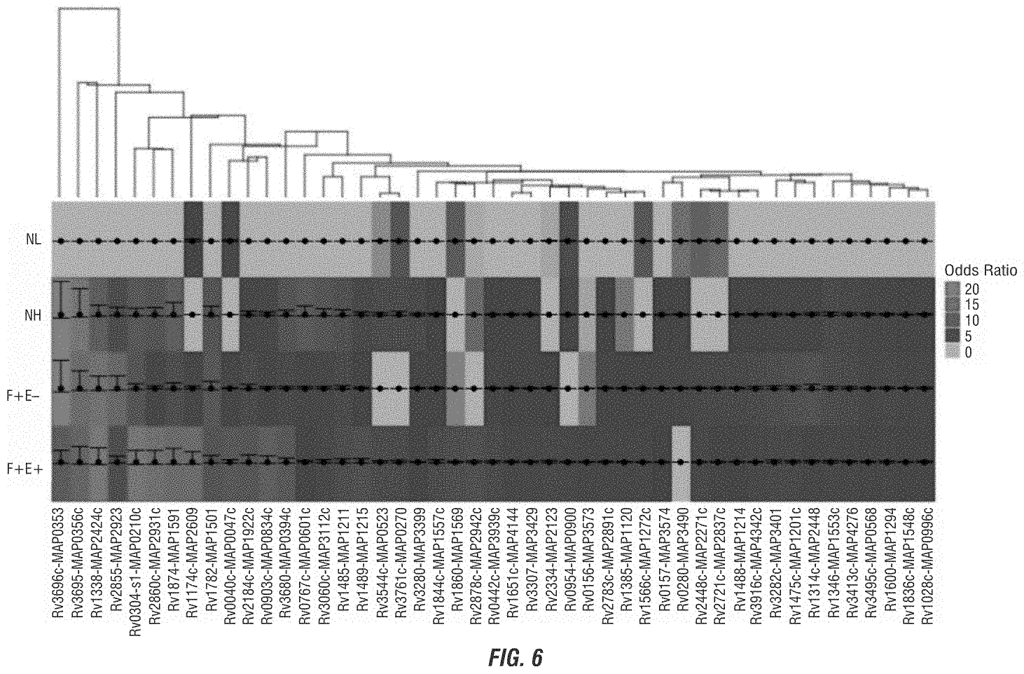

Among the 100 significantly reactive MTB proteins, there were 91 proteins with mean intensities close to or higher than 0.5 and 9 proteins with intensities lower than 0.5. Normalized intensities at 0.5 indicated an approximately 41% higher signal than background where 0 represents the equivalence with background intensities. Among these 9 proteins, mean intensities in the NL group were near 0 and mean intensities in infected groups were more likely to be significantly higher even mean intensities are slightly increased when compared to NL. Therefore, these 9 proteins were excluded to avoid false positives. For the remaining 91 proteins identified in the MTB array, the MAP orthologs were determined based on the comparison of their amino acid sequences and the patterns of antigenicity between the MTB protein identified on the array and the corresponding MAP ortholog. Specifically, for a MAP protein to be considered an ortholog of the identified MTB protein, the amino acid sequence identity must be >40%. However, some proteins, such as Rv0304c-s1 and MAP0210c, which have an overall low identity but show a higher identity in the antigenic regions, are also considered to be MAP orthologs. While the majority of MTB proteins match one single MAP protein, in some cases there are two or more MTB proteins matching the same MAP ortholog, such as Rv0304c & Rv1004c to MAP0210c; Rv1677 & Rv2878c to MAP2942c; Rv1651c & Rv2328 to MAP4144. MAP orthologs were selected from the infected groups based on percent sequence identity and mean intensity values of corresponding MTB proteins on microarrays. For instance, 5 MTB proteins (Rv1753c, Rv0442c, Rv1918c, Rv1917c, and Rv3350c) match MAP3939c with identities ranging from 58.2% to 72.2% at the amino acid level (FIG. 5). These 5 MTB orthologs are PPE family proteins with an identity between 49% and 71% between each other. However, Rv0442c is the most closely related ortholog with an amino acid sequence identity of 72.2% and the highest mean intensity. The MAP3939c and Rv0442c also showed similar antigenicity patterns (FIG. 5 and FIG. 6). A total of 73 MAP orthologs were determined from initial 100 significant MTB antigens identified from MTB array. The logistic regression analysis was applied to 73 MTB orthologs and ordinal logical regression models were fit. In each model the baseline has an odds ratio of 1.0, and all the other categories have odds ratios greater than 1.0, compared to the baseline. Among 73 proteins, there are 47 proteins having significantly different normalized intensity values in at least one group (p<0.05). The remaining 26 antigens did not significantly differ in any of the 4 groups and were excluded as antigens. The 47 antigens were visualized in the heatmap showing the odds ratios for serum reactivity to each antigen among 4 groups (FIG. 6).

### Recognition of Identified Reactive Antigens in Previous Studies

Several MAP orthologs that were identified in the MTB microarray were also recognized in previous studies by other researchers. For instance, the orthologs MAP2609, MAP2942, and MAP0210c were previously characterized as secreted 9, 15, and 34 kDa MAP antigens, which were recognized by antibodies from naturally infected cattle at both clinical and subclinical stages [21]. The ortholog MAP1569 (ModD) was also identified as a secreted protein that was recognized by sera collected from naturally infected cows [22, 23]. The ortholog MAP0834c, a two component system transcriptional regulator, was recognized by sera from naturally MAP infected sheep as a significantly reactive antigen [24]. Another ortholog MAP1272c, an invasion-associated protein, has been identified in several studies as one a promising antigen [24, 25] and recently further characterized on crystal structures, combined with functional assays [26]. The ortholog MAP0900 (P35), a conserved membrane protein, was recognized by 100% of animals including cattle, goats and sheep with Johne's disease in the clinical stage and 75% of cattle in the sub-clinical stage [27], as well as 75% of patients with Crohn's disease [28]. One protein, Rv1411c (ortholog MAP1138c), significantly reactive in F+E+ group but not listed as identified MAP orthologs due to low mean intensities (<0.5), was also recognized in previous studies as immunogenic [29]. Antibody to expressed recombinant protein MAP1138c (P22) was detected in sheep vaccinated by a MAP strain and also in clinical/subclinical cows with Johne's disease [29]. The recombinant P22 (MAP1138c) was able to stimulate significant IFN-γ production in blood of P22-immunized sheep [30]. It needs to be noted that all of the above proteins in previous studies were tested in a relatively small number of infected animals and the majority of animals were tested positive with commercially available ELISA tests. About 90% of identified orthologs with the MTB microarray assays in this study have never been tested for their serological reactivity on a large scale set of serum samples.

### Sensitivity and Specificity of Identified Top Antigens

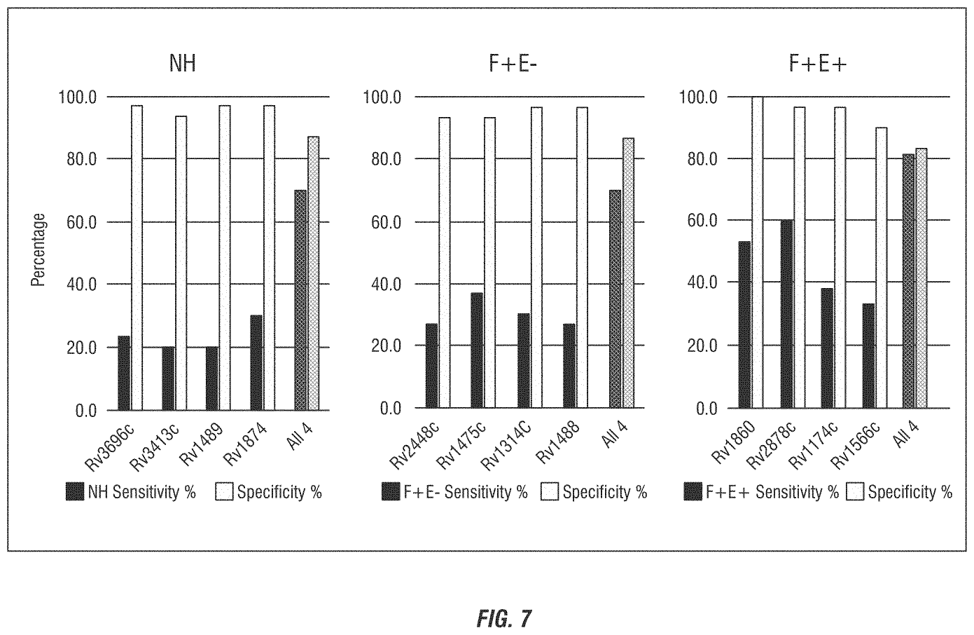

Our goal was to establish a collection of antigens that could be used as a multiplex set to accurately distinguish MAP-infected animals from non-infected animals. To do this, we compared the sensitivity and specificity for each of the 73 identified proteins at both mean+1 standard deviation (1SD) and mean+2SD level. Specificity at the M+1SD cutoff is between 63.3% and 93.3% with a median of 83.3%, and increased to 73.3% to 100.0% with a median of 96.7% at the M+2SD cutoff. Sensitivities for the majority of single proteins were low with median sensitivities of 33.3%, 28.3%, and 30.5% at M+1SD cutoff in NH, F+E−, and F+E+ groups, respectively, and further reduced to 16.7%, 16.7%, and 15.0% at the M+2SD cutoff. Based on comparison of odds ratio and sensitivity/specificity for each protein, we focused on proteins with relatively high sensitivity/specificity and compared different combinations of several proteins to find the best combination with high sensitivity without significantly lowering specificity. For each of group NH, F+E−, and F+E+, we selected a combination of 4 proteins. At the M+1SD cutoff, the sensitivity with the 4 combined proteins significantly increased and reached 80.0%, 85.0%, and 88.3% in the NH, F+E−, and F+E+ groups respectively, however, the specificity dropped from above 90.0% with a single protein to 43.3% and 73.3%, respectively. To avoid false positives, we chose a cutoff at M+2SD level and the sensitivity at each group significantly increased with specificities all above 80.0% (FIG. 7). These results indicate that using a combination of antigens greatly increases the sensitivity in detecting MAP with only a relatively small reduction in specificity.

### Reactivity of MAP Orthologs Confirmed on ELISA

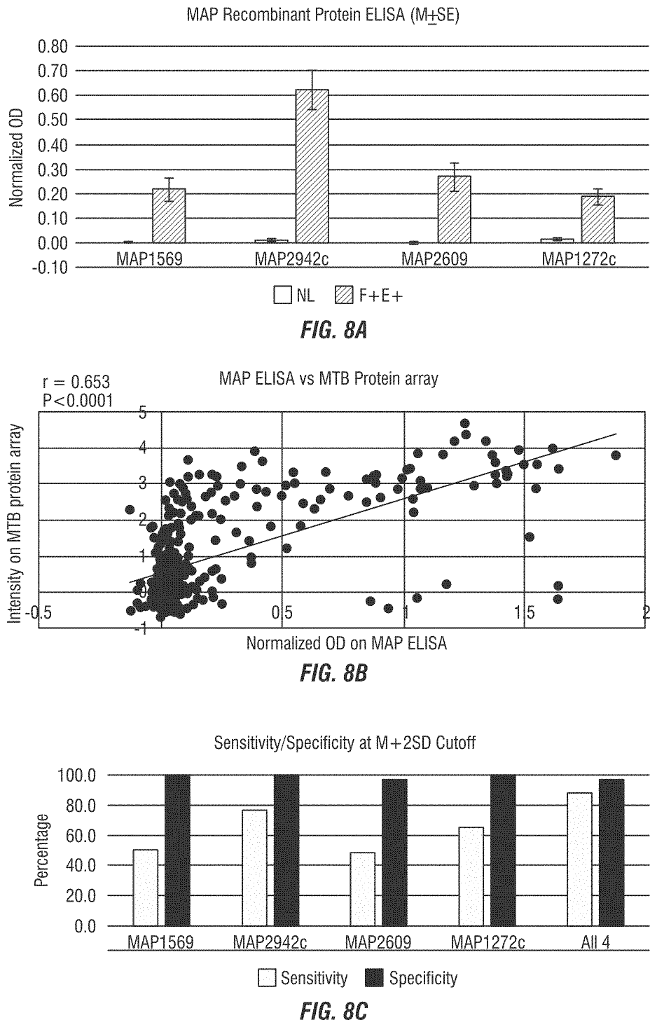

To evaluate if antigens identified with the MTB protein microarray are reactive in infected cows, four recombinant proteins of MAP orthologs (MAP1569, MAP2942c, MAP2609, and MAP1272c corresponding to Rv1860, Rv2878c, Rv1174c and Rv1566c) were selected for ELISA with 90 serum samples including 30 from NL and 60 from F+E+. The identities of these four orthologs between MAP and MTB are from 61.8% to 77.6%. The normalized OD values in two groups were compared and OD values in F+E+ group were significantly higher than that in NL group with p<0.01 for all 4 antigens (FIG. 8A). This result was consistent with the group comparison in MTB protein array, but the background was much lower in NL group, and the ratio of positive/negative was greatly increased in the MAP ELISA. Correlation between the seroreactivity of antigens on the MAP ELISA and orthologs on the MTB array was also examined. For each serum sample, the normalized OD on MAP ELISA was compared to intensity on MTB array and the correlation coefficient, Pearson's rho, was from 0.395 to 0.796 with the lowest in MAP1569 and the highest in MAP2942c (p value <0.0001 in each of the antigens). FIG. 8B showed correlation among all 4 proteins (rho=0.653, p<0.0000001), indicating strong correlation between serological reactivity of infected cows to MTB antigen and MAP orthologs. These data suggest that MTB orthologs on the MTB arrays react to serum from MAP-infected cows in a manner similar to MAP ELISA with MAP recombinant proteins. Based on ELISA data, the sensitivity and specificity for detection of infection was examined and compared with that in MTB protein array at M+1SD and M+2SD cutoff levels. At M+1SD cutoff, the sensitivity on each individual antigen ranged from 55.0% to 81.7% with specificity 83.3% to 96.7%. With 4 antigens combined, the sensitivity increased to 96.7%, but specificity was reduced to 70.0%. At M+2SD cutoff, although sensitivity of each individual antigen was reduced (48.3%-76.7%), the specificity ranged from 96.7% to 100%. With 4 antigens combined, sensitivity increased to 88.3% with specificity 96.7%. Compared to the MTB array on these 4 antigens, MAP ELISA displayed higher sensitivity and specificity. The consistency of group comparison and strong correlation between MTB array and MAP ELISA indicate that antigenic orthologs identified on MTB protein array with serum samples from cows are capable of distinguishing infected cows from uninfected cows.

### Discussion

Generally, determination of significantly reactive antigens for recombinant proteins is based on the comparison of serological reactivity of infected animals to uninfected animals. Usually, when an animal tests MAP negative for both fecal (culture or PCR) and ELISA (serum or milk), we consider the animal to be not infected. However, in this case, the uninfected status may not be true because MAP infection at the tissue level is unknown. Several studies have shown that cattle determined not to be shedding based on either fecal culture or PCR were later found to be MAP-infected in their tissues at the slaughterhouse. Whitlock et al. reported that more than 30% of fecal culture negative cattle from moderately infected herds (fecal culture positive ranging between 5% and 15%) have infected tissues taken at the time of slaughter [31]. Another study comparing MAP culture and PCR in fecal and tissue samples from intestine and the mesenteric lymph node found that MAP was detected by PCR and isolated from tissues in some cattle testing fecal negative [32]. A recent study compared the lymphatic fluid, fecal material, and antibodies from serum and milk samples (ELISA) for detection of MAP infection in cows. The results showed that more than two thirds of animals with a positive lymph result were negative in all fecal and ELISA tests and only 7% of the animals with positive lymph-PCR were also positive in all other tests [33]. Taken together, these results indicate that some animals with negative fecal and ELISA tests are not a true negative.

In this study, 60 samples with both fecal and ELISA negative results were divided into two groups, NL and NH, according to the prevalence of the farms where the samples were collected. By comparing the means of normalized intensities between these two groups, we identified 27 proteins with significantly higher reactivity. Among the 27 identified proteins, two thirds were also shared with F+E−, F+E+, or both, indicating the proteins identified in NH are likely to be true antigens. We hypothesized that cows in the NH group may not be true negatives and were probably in early stage of infection. We found that if NH was used for reference, only 31% and 34% of reactive antigens were identified in the F+E− and F+E+ groups respectively, as compared when NL was used as a reference. Because it is important to select true negatives as a reference to identify reactive antigens in the infected groups of animals we analyzed our data set using NL as the reference.

We hypothesized the stages of infection in the cows as follows; NL=Uninfected; NH=Early; F+E−=Middle; and F+E+=Late stage of infection. There is no significant difference in average lactation number among the 4 groups: NL is 3.13 (SD=1.46), NH 2.93 (SD=+1.08), F+E− 2.95 (SD=1.06), F+E+ 3.32 (SD=+1.40). All infected cows are likely to be in the sub-clinical stage because there were no clinical signs of Johne's disease recorded. As mentioned above, NH showed a different profile of serological reactivity to recombinant proteins compared to NL despite the negative results from the fecal exam and commercial ELISA. Therefore, we speculated that cows in NH were infected with MAP at the early stage. At this stage, serological reaction with traditional commercial ELISA is unlikely to be detected according to experiments in cows with established MAP infection. The time required for seroconversion in experimentally infected calves detectable by commercially available ELISAs is between 10 and 28 months [34]; and it may take possibly longer in naturally infected animals. Although animals generally shed MAP in their feces before seroconversion, the chance of detecting MAP shedding at this stage is very low due to intermittent shedding as observed in many experimentally infected animals [35]. A comparative investigation on cows in slaughterhouses demonstrated viable MAP (or MAP DNA) isolated from mesenteric lymph nodes and intestinal tissues but not from feces in some cows [32], indicating that negative fecal tests could not exclude infection in gut tissue. The other two infected groups, F+E− and F+E+, were both positive in fecal testing, with or without positive ELISA, but the bacterial burden in feces was significantly different (P<0.001). According to two fecal qPCR tests, the average Ct values in F+E− were 35.6 (SD=±2.7) and 37.7 (SD=±2.5), compared to 26.7 (SD=4.1) and 29.8 (SD=4.2) in F+E+, indicating that the MAP burden in the F+E+ group was at least 100 times higher than the F+E− group. The cows in F+E− were considered to be low shedders while the F+E+ group contained high shedders. Based on the quantity of fecal MAP shedding and serological reactivity (ELISA) results, it is reasonable to assign cows in the F+E− group as middle stage infection and the F+E+ group as late stage infection. In previous studies, cows have usually been classified as negative, sub-clinical, and clinical. In this study, we further divided sub-clinical into early, middle, and late stages and identified unique and shared reactive antigens at these different stages of infection.

Currently available ELISA methods are not able to detect serological reactivity during early infection, as shown previously and confirmed in this study and ELISA results only appear as positive during the later stages of infection. With the completion of the genome sequence of MAP K10, it became possible to identify potentially antigenic proteins at a full proteome scale [6], and follow-up studies focusing on the ontogeny of the humoral response to MAP led to identification of antigens marking the early stages of infection. For instance, in experimentally infected cattle, some recombinant MAP proteins were identified on the basis of the humoral immune response as early as 70 days after infection [36]. These identified antigens were also recognized by sera from naturally infected cattle in the sub-clinical stage of Johne's disease. Other studies with MAP experimentally infected cattle showed that the antibody against the recombinant protein (MAP1197) was detected 2-7 months earlier than a commercially available ELISA kit and even earlier than shedding in some cattle [14]. In naturally infected sheep with mild histological lesions of paratuberculosis, more than half of the serum samples had detectable antibody responses against recombinant MAP proteins, but no response to the commercial ELISA [13]. Although promising, a comprehensive identification of the most promising antigens during early stages of MAP infection was limited by several factors. First, there was no well-characterized collection of serum samples from naturally infected animals available to validate recombinant proteins and naturally infected host animals since these were often not classified by different stages of sub-clinical infection. Second, it is difficult to screen large numbers of recombinant proteins using standard ELISA or western blotting techniques, as performed in previous studies. To overcome these limitations, during this investigation, we used a total 180 serum samples from well characterized animals for screening of ˜4,000 recombinant MTB proteins and identified reactive antigens at stages of early, middle, and late infection. A total of 12 and 23 MAP orthologs were identified in the NH and F+E− groups, respectively, although all cows in these two groups showed negative serological reaction based on commercial ELISA tests on both serum and milk samples. Fifty-three MAP orthologs were identified from F+E+. We compared the sensitivity and specificity of each identified ortholog and tested if the sensitivity increased without losing specificity. As a result, 4 proteins were selected from each group and combining these 4 antigens increased sensitivity without an appreciable loss in specificity. As shown in FIG. 7, sensitivity increased from 20.0-30.0% with a single antigen to 60.0% with the 4 combined in NH, 26.7-36.7% to 63.3% in F+E−, and 33.0-60.0% to 81.7% in F+E+. Compared to results with commercial ELISA methods, there is considerable advantage for detection of reactive antigens with recombinant proteins during the early and middle stages of infection because there was no detectable antibody response against a crude mixture of antigens with commercial methods. However, at the late stage of infection (F+E+) with high shedding levels, commercial ELISA methods showed higher sensitivity as compared to recombinant proteins. This is consistent with previous studies showing that ELISA has a higher sensitivity in animals with a heavier bacterial load (high shedders) compared to low shedders [31]. Combined recombinant proteins showed increased sensitivity for detection of infected cows in this study and we will plan to test more combination of different proteins to improve the detection of infected animals in the future study.

While eight of the significantly reactive antigens identified with the MTB protein array in our current investigation have also previously been reported to be recognized in sera from animals with subclinical and clinical infection [21-27, 29], a majority of the others have not, suggesting that the protein microarray approach has considerably utility for diagnostic antigen discovery. Further, our analyses suggest that the serological reactivity to MAP recombinant proteins with ELISA is consistent with reactivity to MTB orthologs on MTB arrays with a strong correlation between reactivity to MTB orthologs on the protein array and to MAP proteins on ELISA. These results are consistent with our earlier finding of concordance in scale and direction of serological reactivity between MTB and MAP arrays [17].

A majority of MAP proteins that were previously described as “non-antigenic” were also not reactive in the MTB array, having either very low mean intensities or no significant difference between the infected and control groups. On the other hand, some of the proteins previously recognized as sero-reactive failed to be recognized as significantly reactive on the MTB arrays. This could be due to the fact that: (i) the previously recognized MAP proteins had no homologs in MTB; (ii) identity of orthologs is too low for a MTB spot to be recognized by antibodies against MAP orthologs; (iii) since there was only a small number of samples tested in most of the previous studies, the results may not accurately reflect the true status; or (iv) some antigens may have been identified in experimentally infected animals and there might be differences in serological response between natural and experimentally infected animals. The utility of the MTB array is limited when MAP proteins are either not represented or have low levels of similarity to their MTB orthologs. For example, MAP2121c, a 35 kDa major membrane protein (MMP) was identified as a reactive antigen in several MAP studies [36-39], has no ortholog in MTB. Similarly, a cluster of MAP proteins from MAP0851-0865 have no orthologs in MTB and are thus not included on the array even though several proteins in the cluster were identified as antigenic in previous studies [12, 40]. Example 3 herein overcomes this potential issue of the MTB array by identifying additional antigens with a MAP protein microarray.

It is important to note that all 8 proteins identified both in this MTB array study and previous studies were only found in the F+E+ except for one (MAP0210c, Rv0304), which was also recognized in cows from the NH and F+E− groups. This is probably because the majority of infected animals used in previous studies were at clinical or late sub-clinical stages, and the majority of cows in this study (such as NH and F+E− groups) were at early or middle stages of infection. About 80% of identified orthologs with the MTB microarray in this study have never been tested in previous studies for their serological reactivity with a robust and representative serum bank, and many of these candidates will need to be expressed and added to the MAP protein array for future studies.

In conclusion, the results of our studies have led to the identification of a large number of promising candidate antigens that provide a strong framework for the future development of the next generation of highly sensitive and specific diagnostic assays for the diagnosis of early MAP infection in cattle and other susceptible hosts as further shown in Example 2.

### REFERENCES

1. Cocito C, Gilot P, Coene M, de Kesel M, Poupart P, Vannuffel P. Paratuberculosis. Clin Microbiol Rev. 1994; 7(3):328-45. Epub 1994/07/01.
2. Ott S L, Wells S J, Wagner B A. Herd-level economic losses associated with Johne's disease on US dairy operations. Prev Vet Med. 1999, 40(3-4):179-92.
3. USDA-APHIS. Johne's Disease on U.S. Dairies, 1991-2007. Ft. Collins, Colo.: Fort Collins: USDA-APHIS-VSCEAH. #N521.0408; 2008.
4. Clarke C J. The pathology and pathogenesis of paratuberculosis in ruminants and other species. J Comp Pathol. 1997; 116(3):217-61.
5. Stewart D J, Vaughan J A, Stiles P L, Noske P J, Tizard M L, Prowse S J, et al. A long-term study in Merino sheep experimentally infected with Mycobacterium avium subsp. paratuberculosis: clinical disease, faecal culture and immunological studies. Vet Microbiol. 2004; 104(3-4):165-78.
6. Li L, Bannantine J P, Zhang Q, Amonsin A, May B J, Alt D, et al. The complete genome sequence of Mycobacterium avium subspecies paratuberculosis. Proc Natl Acad Sci USA. 2005; 102(35):12344-9. Epub 2005/08/24.
7. He Z, De Buck J. Localization of proteins in the cell wall of Mycobacterium avium subsp. paratuberculosis K10 by proteomic analysis. Proteome Sci. 2010; 8:21. Epub 2010/04/10.
8. Leroy B, Roupie V, Noel-Georis I, Rosseels V, Walravens K, Govaerts M, et al. Antigen discovery: a postgenomic approach to paratuberculosis diagnosis. Proteomics. 2007; 7(7):1164-76.
9. McNamara M, Tzeng S C, Maier C, Zhang L, Bermudez L E. Surface proteome of “Mycobacterium avium subsp. hominissuis” during the early stages of macrophage infection. Infect Immun. 2012; 80(5):1868-80.
10. Cho D, Collins M T. Comparison of the proteosomes and antigenicities of secreted and cellular proteins produced by Mycobacterium paratuberculosis. Clin Vaccine Immunol. 2006; 13(10):1155-61.
11. Hughes V, Bannantine J P, Denham S, Smith S, Garcia-Sanchez A, Sales J, et al. Immunogenicity of proteome-determined Mycobacterium avium subsp. paratuberculosis-specific proteins in sheep with paratuberculosis. Clin Vaccine Immunol. 2008; 15(12):1824-33.
12. Bannantine J P, Paustian M L, Waters W R, Stabel J R, Palmer M V, Li L, et al. Profiling bovine antibody responses to Mycobacterium avium subsp. paratuberculosis infection by using protein arrays. Infect Immun. 2008; 76(2):739-49. Epub 2007/11/28.
13. Gumber S, Taylor D L, Whittington R J. Evaluation of the immunogenicity of recombinant stress-associated proteins during Mycobacterium avium subsp. paratuberculosis infection: Implications for pathogenesis and diagnosis. Vet Microbiol. 2009; 137(3-4):290-6. Epub 2009/02/03.
14. Nagata R, Kawaji S, Mori Y. Use of enoyl coenzyme A hydratase of Mycobacterium avium subsp. paratuberculosis for the serological diagnosis of Johne's disease. Vet Immunol Immunopathol. 2013; 155(4):253-8. Epub 2013/08/28.
15. Waters W R, Miller J M, Palmer M V, Stabel J R, Jones D E, Koistinen K A, et al. Early induction of humoral and cellular immune responses during experimental Mycobacterium avium subsp paratuberculosis infection of calves. Infection and Immunity. 2003; 71(9):5130-8.
16. Bannantine J P, Stabel J R, Bayles D O, Geisbrecht B V. Characteristics of an extensive Mycobacterium avium subspecies paratuberculosis recombinant protein set. Protein expression and purification. 201072(2):223-33.
17. Bannantine J P, Campo J J, Li L, Randall A, Pablo J, Praul C A, et al. Identification of Novel Seroreactive Antigens in Johne's Disease Cattle Using the Mycobacterium tuberculosis Protein Array. Clin Vaccine Immunol. 2017.
18. Kunnath-Velayudhan S, Salamon H, Wang H Y, Davidow A L, Molina D M, Huynh V T, et al. Dynamic antibody responses to the Mycobacterium tuberculosis proteome. Proc Natl Acad Sci USA. 2010; 107(33):14703-8. Epub 2010/07/30.
19. Kunnath-Velayudhan S, Davidow A L, Wang H Y, Molina D M, Huynh V T, Salamon H, et al. Proteome-scale antibody responses and outcome of Mycobacterium tuberculosis infection in nonhuman primates and in tuberculosis patients. J Infect Dis. 2012; 206(5):697-705. Epub 2012/06/27.
20. Benjamini Y, Hockberg Y. Controlling the false discovery rate: a practical and powerful approach to multiple testing. Journal of the Royal Statistical Society, Series B 1995; 57(1):289-300.
21. Willemsen P T, Westerveen J, Dinkla A, Bakker D, van Zijderveld F G, Thole J E. Secreted antigens of Mycobacterium avium subspecies paratuberculosis as prominent immune targets. Vet Microbiol. 2006; 114(3-4):337-44. Epub 2006/01/18.
22. Cho D, Shin S J, Talaat A M, Collins M T. Cloning, expression, purification and serodiagnostic evaluation of fourteen Mycobacterium paratuberculosis proteins. Protein expression and purification. 2007; 53(2):411-20. Epub 2007/02/14.
23. Cho D, Sung N, Collins M T. Identification of proteins of potential diagnostic value for bovine paratuberculosis. Proteomics. 2006; 6(21):5785-94.
24. Gurung R B, Begg D J, Purdie A C, Bannantine J P, Whittington R J. Antigenicity of Recombinant Maltose Binding Protein-Mycobacterium avium subsp paratuberculosis Fusion Proteins with and without Factor Xa Cleaving. Clinical and Vaccine Immunology. 2013; 20(12):1817-26.
25. Bannantine J P, Lingle C K, Stabel J R, Ramyar K X, Garcia B L, Raeber A J, et al. MAP1272c encodes an NpC/P60 protein, an antigen detected in cattle with Johne's disease. Clin Vaccine Immunol. 2012; 19(7):1083-92. Epub 2012/05/18.
26. Bannantine J P, Lingle C K, Adam P R, Ramyar K X, McWhorter W J, Stabel J R, et al. NlpC/P60 domain-containing proteins of Mycobacterium avium subspecies paratuberculosis that differentially bind and hydrolyze peptidoglycan. Protein science: a publication of the Protein Society. 2016; 25(4):840-51.
27. El-Zaatari F A, Naser S A, Graham D Y. Characterization of a specific Mycobacterium paratuberculosis recombinant clone expressing 35,000-molecular-weight antigen and reactivity with sera from animals with clinical and subclinical Johne's disease. Journal of clinical microbiology. 1997; 35(7):1794-9.
28. Shafran I, Piromalli C, Decker J W, Sandoval J, Naser S A, E I-Zaatari F A. Seroreactivities against Saccharomyces cerevisiae and Mycobacterium avium subsp. paratuberculosis p35 and p36 antigens in Crohn's disease patients. Digestive diseases and sciences. 2002; 47(9):2079-81.
29. Dupont C, Thompson K, Heuer C, Gicquel B, Murray A. Identification and characterization of an immunogenic 22 kDa exported protein of Mycobacterium avium subspecies paratuberculosis. J Med Microbiol. 2005; 54(Pt 11):1083-92. Epub 2005/09/30.
30. Rigden R C, Jandhyala D M, Dupont C, Crosbie-Caird D, Lopez-Villalobos N, Maeda N, et al. Humoral and cellular immune responses in sheep immunized with a 22 kilodalton exported protein of Mycobacterium avium subspecies paratuberculosis. J Med Microbiol. 2006; 55(Pt 12):1735-40. Epub 2006/11/17.
31. Whitlock R H, Wells S J, Sweeney R W, Van Tiem J. ELISA and fecal culture for paratuberculosis (Johne's disease): sensitivity and specificity of each method. Vet Microbiol. 2000; 77(3-4):387-98.
32. Pribylova R, Slana I, Kralik P, Kralova A, Babak V, Pavlik I. Correlation of Mycobacterium avium subsp. paratuberculosis counts in gastrointestinal tract, muscles of the diaphragm and the masseter of dairy cattle and potential risk for consumers. International journal of food microbiology. 2011; 151(3):314-8.
33. Khol J L, Pinedo P J, Buergelt C D, Neumann L M, Rae D O. Lymphatic fluid for the detection of Mycobacterium avium subsp. paratuberculosis in cows by PCR, compared to fecal sampling and detection of antibodies in blood and milk. Vet Microbiol. 2014; 172(1-2):301-8.
34. Milner A R, Lepper A W, Symonds W N, Gruner E. Analysis by ELISA and Western blotting of antibody reactivities in cattle infected with Mycobacterium paratuberculosis after absorption of serum with M. phlei. Research in veterinary science. 1987; 42(2):140-4.
35. Plattner B L, Chiang Y W, Roth J A, Platt R, Huffman E, Zylstra J, et al. Direct inoculation of Mycobacterium avium subspecies Paratuberculosis into ileocecal Peyer's patches results in colonization of the intestine in a calf model. Veterinary pathology. 2011; 48(3):584-92.
36. Bannantine J P, Bayles D O, Waters W R, Palmer M V, Stabel J R, Paustian M L. Early antibody response against Mycobacterium avium subspecies paratuberculosis antigens in subclinical cattle. Proteome Sci. 2008; 6:5. Epub 2008/01/30.
37. Shin S J, Yoo H S, McDonough S P, Chang Y F. Comparative antibody response of five recombinant antigens in related to bacterial shedding levels and development of serological diagnosis based on 35 kDa antigen for Mycobacterium avium subsp. paratuberculosis. J Vet Sci. 2004; 5(2):111-7. Epub 2004/06/12.
38. Bannantine J P, Huntley J F, Miltner E, Stabel J R, Bermudez L E. The Mycobacterium avium subsp. paratuberculosis 35 kDa protein plays a role in invasion of bovine epithelial cells. Microbiology. 2003; 149(Pt 8):2061-9. Epub 2003/08/09.
39. Bannantine J P, Radosevich T J, Stabel J R, Berger S, Griffin J F, Paustian M L. Production and characterization of monoclonal antibodies against a major membrane protein of Mycobacterium avium subsp. paratuberculosis. Clin Vaccine Immunol. 2007; 14(3):312-7. Epub 2007/02/03.
40. Paustian M L, Amonsin A, Kapur V, Bannantine J P. Characterization of novel coding sequences specific to Mycobacterium avium subsp. paratuberculosis: implications for diagnosis of Johne's Disease. Journal of clinical microbiology. 2004; 42(6):2675-81.

### Example 2: Early Detection of Mycobacterium avium Subsp. paratuberculosis Infection in Cattle with Multiplex-Bead Based Immunoassays

Johne's disease (JD) is a chronic granulomatous intestinal inflammatory disease that results from infection with Mycobacterium avium subspecies paratuberculosis (MAP) [1]. Although animals are infected early in life through ingestion of bacilli via the fecal-oral route or from colostrum, JD takes several years to manifest [2,3]. During this extremely long sub-clinical phase, infected animals are continuously or intermittently shedding the pathogen into the environment and spreading the disease. JD is recognized as a serious animal health problem in domesticated ruminants including dairy and beef cattle, sheep, and goats, resulting in more than $200 million in annual losses to the US dairy industry with additional losses incurred in other species [4]. The current diagnostic methods of MAP infection including fecal tests and serological immunoassays (ELISA) have been limited in detection of infected from non-infected animals during early infection because it is very difficult to reliably identify infected animals that are intermittently shedding with fecal tests and currently available ELISA assays have low sensitivity in detecting animals with subclinical infection, and only about one third of MAP-infected cows are detected by current ELISA assays in longitudinal studies [5,6].

Current ELISA assays use relatively crude cellular extracts that share antigens with other common mycobacteria and need cumbersome pre-absorption steps in order to ensure specificity [7]. However, this also results in a considerable decrease in analytical and diagnostic sensitivity [8], highlighting the need for more sensitive, high-throughput screening assays to identify MAP-infected animals during the early, subclinical phase. Since the first complete MAP genome sequence was published [9], many studies with recombinant MAP proteins have been conducted to identify potential candidates for use as diagnostic antigens that could distinguish animals with mild or early MAP infection from those uninfected [10-16]. We recently screened a set of well-characterized serum samples using a whole proteome microarray from Mycobacterium tuberculosis (MTB), and several promising candidate antigens were identified from these studies as immunogenic during MAP infection [17, Example 1]. These antigens need to be further evaluated for the development of a high-throughput, diagnostic immunoassay.

One commonly used high-throughput screen technique is fluorescent bead-based multiplex immunoassay that involves 100 distinctly color-coated bead sets created by the use of two fluorescent dyes (internal dye and reporter dye) at distinct ratios (e.g. LUMINEX@, luminexcorp.com). Each bead set can be coated with an antigen specific to a particular assay, allowing the capture and detection of a specific analyte from a given sample [18]. Such multiplex immunoassays have been successfully applied to quantify antibodies to pathogens such as Borrelia burgdorferi, Chlamvdia trachomatis, Streptococcus pneumoniae, Haemophilus influenza, Moraxella catarrhalis, and equine herpesvirus in human and animal serum samples [19-22].

The aim of this study was to evaluate candidate antigens that can be used to develop a bead-based multiplex immunoassay which reliably identifies diagnostic markers in both serum and milk samples from MAP infected animals. To our knowledge, no bead-based multiplex assay has yet been developed for detection of MAP infection. Here, we describe the development of a multiplex immunoassay for simultaneous detection of antibodies specific to six candidate recombinant MAP proteins. Five of these proteins (MAP1272c, MAP1569, MAP2609, MAP2942c and MAP1201c+2942c fusion protein) were selected because they displayed the highest levels of sensitivity and specificity in our previous protein array studies [17; Example 1]. Additionally, MAP2121c was selected based on previous studies that showed significant reactivity to samples from infected animals in previous ELISA studies [10,23] although it was not shown in the MTB array due to the absence of an ortholog in MTB [17; Example 1]. The results show that multiplex bead-based assays reliably identify cows with MAP infection using both serum and milk samples, even during early stages of infection in animals that were fecal test positive but negative based on widely used commercial ELISAs.

### Materials and Methods

### Bovine Serum and Milk Samples

All serum and milk samples were collected as part of the Johne's Disease Integrated Program (JDIP, mycobacterialdiseases.org) diagnostic standards sample collection project and have been previously assayed for fecal and ELISA, as described [17; Example 1]. Animal use protocols were approved by the Pennsylvania State University ISCUC under numbers 34626 and 43309. In brief, the serum and milk samples used in these studies were collected from cows housed in 13 dairy farms from 4 states: California, Georgia, Minnesota, and Pennsylvania. The herd size ranged from 66 to 1,400, and prevalence of JD ranged from 0 to 53.30% based on serum ELISA tests conducted prior to sample collection. All herds were negative for bovine TB. Each cow was tested for level of MAP shedding in feces as well as serological reactivity. MAP shedding was determined by fecal culture using Herrold's solid medium (HEYM) and two different liquid culture medium systems, BACTEC MGIT and Trek (Becton, Dickinson and Company, Franklin Lakes, N.J.); all fecal cultures were confirmed by acid fast staining and PCR tests. Fecal qPCR assays were performed for each animal with the LT TaqMan (ThermoFisher, Waltham, Mass.) and Tetracore (Tetracore, Rockville, Md.) assays. Serum and milk ELISA tests were performed using both the IDEXX kit (IDEXX Laboratories, Inc., ME) and the ParaChek (ThermoFisher, Waltham, Mass.) according to the manufacturers' instructions. Samples were selected from 180 cows that were stratified into 3 groups as listed in the table: both fecal and ELISA tests negative, and collected from the herds with previously observed JD prevalence of 0% (NL, n=60); fecal tests positive and ELISA test negative (F+E−, n=60); and both fecal and serological tests positive (F+E+, n=60). Serum samples from all 180 cows and milk samples from 90 out of 180 cows (n=30 per group) were tested in this study.

### Preparation of Recombinant Proteins

The 6 recombinant MAP proteins selected in this study were expressed as maltose binding protein (MBP) fusion proteins because previous studies demonstrated higher yields as compared to six-His tag clones [24]. The full-length coding sequences for 5 of the 6 genes were amplified from MAP K-10 genomic DNA with 5′ primer containing an XbaI and 3′ primer a Hind III restriction site and cloned into the pMAL-c5 translational fusion expression vector (New England Biolabs, Beverly, Mass., USA). The MAP1201c+2942c was chemically synthesized, amplified and cloned in a manner similar to the other 5 genes. The vector and amplification products were each digested with XbaI and HindIII, followed by overnight ligation at 4° C. The products were transformed into E. coli DH5a and selected on LB agar plates containing 0.10 mg/ml ampicillin. Drug-resistant colonies were screened by PCR and plasmid DNA was sequenced to confirm the presence of the correct insert in each clone [24]. These MBP-tagged recombinant proteins were expressed by induction of 1.0-liter LB broth cultures with 0.3 mM isopropyl-β-d-thiogalactopyranoside (Sigma Chemical Company, St. Louis, Mo.) for 2.5 h with shaking at 37° C. E. coli cells were harvested by centrifugation at 4,000×g, re-suspended and subjected to a freeze-thaw cycle at −20° C. and sonication. The resulting extracts were purified by affinity chromatography with an amylose resin as per the manufacturer's instructions (New England Biolabs). Purified protein yields are determined from eluted fractions with a NanoDrop spectrophotometer set at 280 nm. The most concentrated fractions were pooled and dialyzed with three exchanges of PBS at 4° C. Purified protein aliquots were stored at −20° C. after protein yield was reassessed by a modified Lowry assay using bovine serum albumin (BSA) as the standard. Each recombinant protein was further evaluated by using GelCode blue (Pierce Biotechnology Inc., Rockford, Ill.)-stained SDS-PAGE gels to assess purity and expected sizes [24].

### Coupling of Recombinant MAP Proteins to Fluorescent Beads

A total of 100 μg of each purified recombinant MAP protein was coupled to fluorescent beads (Luminex, Austin, Tex.) at room temperature according to the manufacturer's instructions. MAP1272c was coupled to bead 33, MAP1569 to 34, MAP2121c to 35, MAP2942c to 36, MAP2609 to 37, and MAP1201c+2942c to 38. All centrifugation steps were performed at 14,000×g for 4 minutes (min). In brief, the beads were resuspended by vortexing and sonication for 20 seconds. For activation, 5×106 beads were washed once in deionized H₂O. Beads were resuspended in 80 μl of 100 mM sodium phosphate buffer, pH 6.2 and 10 μl of Sulfo-NHS (50 mg/ml) and 10 μl 1-ethyl-3-[3-dimethylaminopropyl] carbodiimide hydrochloride (EDC, 50 mg/ml, both from Pierce Biotechnology Inc., Rockford, Ill.) were added and incubated for 20 min. The beads were then washed twice with 50 mM 2-[N-morpholino] ethanesulfonic acid pH 5.0 (MES) and resuspended in MES solution. These activated beads were used for MAP antigen coupling using 100 μg of each antigen. The coupling of the MAP antigens was performed for three hours with rotation. After coupling, the beads were resuspended in blocking buffer (PBS with 1% (w/v) BSA and 0.05% (w/v) sodium azide) and incubated for 30 min. The beads were washed three time in PBS with 0.1% (w/v) BSA, 0.02% (v/v) Tween 20 and 0.05% (w/v) sodium azide (PBS-T), counted and stored in the dark at 2-8° C.

### Luminex Multiplex Assay

Beads coupled with MAP antigens were sonicated, mixed and diluted in blocking buffer to a final concentration of 1×10⁵ beads/ml each. For the assay, 5×10³ beads/antigen were used per microtiter well. Serum samples were diluted 1:400 and milk samples were diluted 1:2 in blocking buffer. In addition to the samples, a set of three previously determined (NL, F+E− and F+E+) serum and milk samples were run on each plate together with a buffer control. These standard and blank samples were used as inter-assay and background controls. Millipore Multiscreen HTS plates (Millipore, Danvers, Mass.) were soaked with PBS-T using a ELx50 plate washer (Biotek Instruments Inc., Winooski, Vt.) for 2 min. The solution was aspirated from the plates and 50 μl of each diluted standard serum or milk samples were applied to the plates. Then, 50 μl of bead solution was added to each well and incubated for 30 min on a shaker at room temperature. Then, the plate was washed with PBS-T, and 50 μl of biotinylated goat anti-bovine IgG (H+L) detection antibody (Jackson Immunoresearch Laboratories, West Grove, Pa.) diluted 1:1,000 in blocking buffer was added to each well and incubated for 30 min as above. After washing, 50 ml of streptavidin-phycoerythrin (Invitrogen, Carlsbad, Calif.) diluted 1:100 in blocking buffer was added. Plates were incubated for 30 min as above and washed. The beads were resuspended in 100 ml of blocking buffer and the plate was placed on the shaker for 15 min. The assay was analyzed in a Luminex 200 instrument (Luminex Corp., Austin, Tex.). The data were reported as median fluorescent intensities (MFIs).

### Recombinant MAP Protein ELISA

Assays were conducted with serum samples from NL (n=30) and F+E+ groups (n=60) using 6 recombinant MAP proteins that were applied in the multiplex assays. The procedure was adapted from the previously described protocol [25] with a minor modification. ELISA 96-well microplates were coated with 50 μl/well of MBP-tagged recombinant MAP protein (1 μg/ml) or MBP/LacZ fusion protein (0.5 μg/ml) in carbonate/bicarbonate buffer [0.1 M pH 9.6]. Plates were sealed and incubated overnight at 4° C., then washed three times with 1×PBS, pH 7.4 containing 0.1% Tween 20 (PBS-T). Wells were blocked by adding 200 μl of PBS-T containing 1% bovine serum albumin (PBS-T-BSA) and incubated at room temperature for 1 hour before washing the plate three times with PBS-T. Serum samples diluted 1:250 in PBS-T-BSA were added to each well (100 μl) and incubated at room temperature for 1 hour before washing six times with PBS-T. Then anti-goat IgG peroxidase conjugate (Vector Labs, Burlingame, Calif., USA) diluted 1:10,000 in PBS-T-BSA was added to all wells (100 μl) and incubated at room temperature for 1 hour before the plates were again washed six times with PBS-T. Finally, 100 μl/well of tetra methylbenzidine (TMB) SureBlue solution (KPL, Gaithersburg, Md., USA) was added and the reaction incubated for 10-15 minutes at room temperature with no light, before the reaction was stopped with 100 μl/well of 1.0 N HCl solution. The spectrophotometric reading of all wells was performed at 450 nm using a PowerWave XS2 microplate reader (BioTek, Winooski, Vt., USA). The OD value of each sample was normalized by [sample OD−MBP/LacZ OD] to eliminate the background produced by the non-specific binding.

### Statistical Analysis

The group comparison was conducted using one-tailed Mann-Whitney U tests with a significance level at p<0.05 (also called the Wilcoxon Rank-Sum test) to compare MFI values in serum and milk assays in F+E− and F+E+ groups as compared to the NL (socscistatistics.com/tests/mannwhitney/). P-value adjustments were made because multiple statistical tests were performed on the same sample set (e.g. set 1=NL vs. F+E−, set 2=NL vs. F+E+): a Bonferroni correction was applied to alpha (0.05/(number of tests performed)). To determine the sensitivity and specificity for each antigen within the multiplex assay, a Receiver Operating Characteristic (ROC) curve was generated using the ROCR package in the R program (R-project.org/). The cutoffs for sensitivity and specificity were based on maximum Youden Index (J=Se+Sp−1) [26]. The agreement between serum and milk reactivity to each antigen (MFI) was analyzed with Spearman rank correlation (socscistatistics.com/tests/spearman/Default.aspx). The concordance correlation was generated using the Agreement package in R. The Strength of agreement was estimated by Covariance R and the concordance correlation coefficient (CCC) with <0.65 as poor, 0.65-0.8 moderate, 0.8-0.9 substantial, and >0.9 almost perfect.

### Results

### Immunological and Microbiological Assessment of MAP Infection Status

The samples used in our current studies were from animals tested for MAP infection status using ELISA kits (2 for serum and 1 for milk), five fecal assays including three cultures (1 solid and 2 liquid) and two commercial qPCR assays as part of the JDIP diagnostic standards sample collection project (Table 1). All samples from cows in the NL group (from uninfected herds) were negative in each of the eight assays, while 70% of those in the F+E+ group tested positive in all 8 assays, 23.3% positive in 7, and 6.7% in at least 6 of the assays. For animals in the F+E− group, ELISA tests were negative in all cows; while 70% of animals tested positive in at least two of the three fecal culture assays, and the remaining 30% were positive for at least one. The results also showed that 60% of all cows in the F+E− group cows tested positive only with one or more qPCR assays while the remaining 40% had at least 1 positive in culture tests with or without qPCR positive. The fecal qPCR Ct values were significantly lower in the F+E+ group compared to the F+E− group (P<0.001), indicating a considerably higher level of shedding in F+E+ cows (Table 1). The number of lactations and the days in milk (DIM) were comparable in all three groups, and although the values were slightly higher for lactation number and DIM for the F+E− and F+E+ groups compared with the NL group, they were not significant (Table 1).

TABLE 1
Assessment of MAP infection status in 180 samples in this study

| Tests | Statement | NL | F+ E− | F+ E+ |
|---|---|---|---|---|
| Seram ELISA | Pos (%) | 0.00 | 0.00 | 100.00 |
| (IDEXX) | | | | |
| Seram ELISA | Pos (%) | 0.00 | 0.00 | 93.33 |
| (ParaChek) | | | | |
| Milk ELISA | Pos (%) | 0.00 | 0.00 | 91.38 (Susp |
| (IDEXX) | | | | 3.45) |
| Fecal culture | Pos (%) | 0.00 | 10.00 | 85.00 |
| (HEYM) | | | | |
| Fecal culture | Pos (%) | 0.00 | 15.00 | 98.33 |
| (MGIT) | | | | |
| fecal culture | Pos (%) | 0.00 | 33.33 | 100.00 |
| (Trek) | | | | |
| qPCR (LT | Pos (%) | 0.00 | 85.00 | 100.00 |
| TaqMan) | | | | |
| qPCR | Pos (%) | 0.00 | 47.00 (Susp | 98.30 (Susp |
| (Tetracore) | | | 23.00) | 1.70) |
| LT TaqMan Ct | M ± SD | >40 | 35.55 ± | 26.70 ± |
| value | | | 2.74 | 4.05 |
| P value | | | vs NL | vs F+ E− |
| | | | (<0.0001) | (<0.0001) |
| Tetracore Ct | M ± SD | >40 | 37.69 ± | 29.77 ± |
| value | | | 2.52 | 4.16 |
| P value | | | vs NL | vs F+ E− |
| (unpaired, 2 | | | (<0.0001) | (<0.0001) |
| tails) | | | | |
| Lactation | M ± SD | 2.90 ± | 2.95 ± | 3.32 ± |
| number | | 1.27 | 1.06 | 1.40 |
| P value | | | 0.815 | 0.088 |
| (vs NL) | | | | |
| Days in Milk | M ± SD | 166.08 ± | 181.52 ± | 195.72 ± |
| | | 128.45 | 150.39 | 135.97 |
| P value | | | 0.547 | 0.222 |
| (vs NL) | | | | |

### Serum and Milk Multiplex Immunoassays

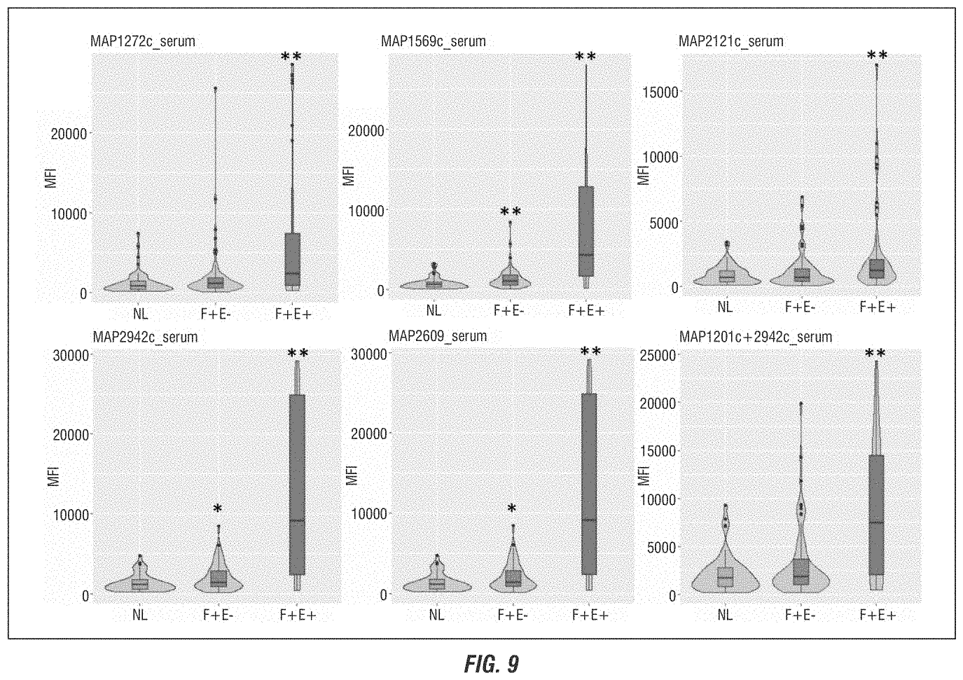

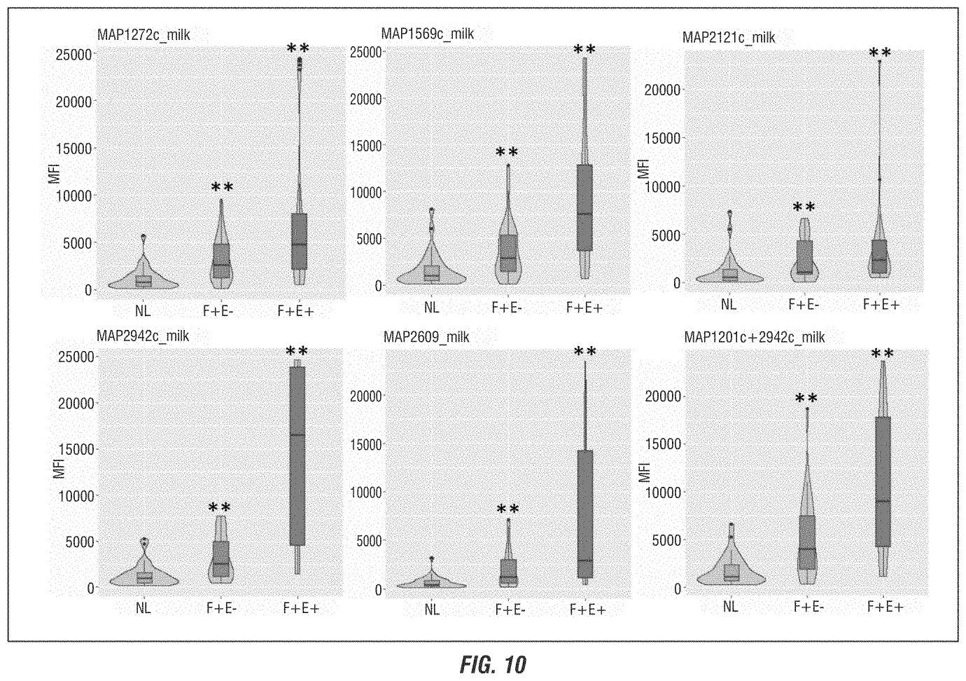

Samples from animals in all three groups, NL, F+E−, and F+E+, were analyzed for all six antigens, for both serum (FIG. 9) and milk (FIG. 10). To assess the immunogenicity of each antigen, the MFI values of samples from animals in the infected groups (F+E+ and F+E−) were compared with those from the control group (NL). The results show that, when considering the 60 serum samples from each of the NL, F+E−, and F+E+ groups, the immunoreactivity of serum from animals in the F+E− group was significantly higher for only 3 of the antigens, MAP1569, MAP2609, and MAP2942c, when compared with the NL (FIG. 9, Table 2). In contrast, immunoreactivity of serum from animals in the F+E+ group was significantly higher for all the six antigens as compared with the NL group (p<0.001). Interestingly, for the milk samples, the results show that the immunogenicity of all six antigens was significantly higher in both F+E− and F+E+ groups (p<0.01) as compared with the NL (FIG. 10, Table 2). The ratio of average MFIs in the F+E− to that in the NL for each of the antigens ranged from 1.4 to 1.7 (median 1.6) in serum and 2.0 to 3.1 (median 2.6) in milk. The highest ratio in serum was for MAP1569 and MAP1272c, and for MAP2609 in milk; the lowest ratio in both serum and milk was MAP2121c. The median ratio for F+E+/NL was 7.3 in serum and 6.5 in milk, MAP1569, MAP2942c, and MAP2609 showed the highest (9.8-9.9) in serum while MAP2942c and MAP2609 showed the highest (11.2-11.5) in milk. Again, MAP2121c showed the lowest ratio in the F+E+ for both serum and milk (Table 2).

TABLE 2
Group comparison of serum and milk MFI values (Mann-Whitney test)

| | | | | | | MAP1201c + |
|---|---|---|---|---|---|---|
| Sample Type | MAP1272c | MAP1569 | MAP2121c | MAP2942c | MAP2609 | 2942c |
| Serum, n = 480 | | | | | | |
| NL (M ± SD) | 1217.8 ± | 836.3 ± | 891.0 ± | 1336.1 ± | 740.2 ± | 2129.4 ± |
| | 1327.7 | 705.2 | 723.4 | 1047.5 | 455.2 | 1906.1 |
| F+E− (M ± SD) | 2086.2 ± | 1410.0 ± | 1246.9 ± | 2086.9 ± | 1207.2 ± | 3257.7 ± |
| | 3674.0 | 1353.2 | 1402.1 | 1696.0 | 1132.1 | 3762.1 |
| F+E+ (M ± SD) | 5877.7 ± | 8168.3 ± | 2343.7 ± | 13113.7 ± | 7334.6 ± | 8683.5 ± |
| | 8035.5 | 8530.8 | 3133.9 | 10854.7 | 7839.0 | 6921.8 |
| Ratio (F+E−/NL) | 1.7 | 1.7 | 1.4 | 1.6 | 1.6 | 1.5 |
| P (F+E− vs NL) | 0.03005 | 0.00042 | 0.1423 | 0.00621 | 0.02275 | 0.11123 |
| Ratio (F+E+/NL) | 4.8 | 9.8 | 2.6 | 9.8 | 9.9 | 4.1 |
| P (F+E+ vs NL) | <.00001 | <.00001 | 0.0002 | <.00001 | <.00001 | <.00001 |
| Milk, n = 90 | | | | | | |
| NL (M ± SD) | 1122.5 ± | 1633.2 ± | 1156.6 ± | 1320.4 ± | 621.5 ± | 1746.5 ± |
| | 1172.3 | 1824.8 | 1619.2 | 1234 | 629.7 | 1538.9 |
| F+E− (M ± SD) | 3168.9 ± | 3588.6 ± | 2350.0 ± | 3220.1 ± | 1900.7 ± | 5160.0 ± |
| | 2331 | 2915.2 | 2105.2 | 2405 | 1873.3 | 4055.4 |
| F+E+ (M ± SD) | 7466.1 ± | 9374.5 ± | 3440.4 ± | 14749.0 ± | 7162.6 ± | 10942.8 ± |
| | 7998.8 | 7042.6 | 4031.8 | 9321.9 | 7564.9 | 7310.4 |
| Ratio (F+E−/NL) | 2.8 | 2.2 | 2.0 | 2.4 | 3.1 | 3.0 |
| P (F+E− vs NL) | 0.00003 | 0.00056 | 0.00154 | 0.00016 | 0.0004 | <.00001 |
| Ratio (F+E+/NL) | 6.7 | 5.7 | 3.0 | 11.2 | 11.5 | 6.3 |
| P (F+E+ vs NL) | <.00001 | <.00001 | <.00001 | <.00001 | <.00001 | <.00001 |

### ROC Analysis of Each HAP Antigen for Serum and Milk Samples

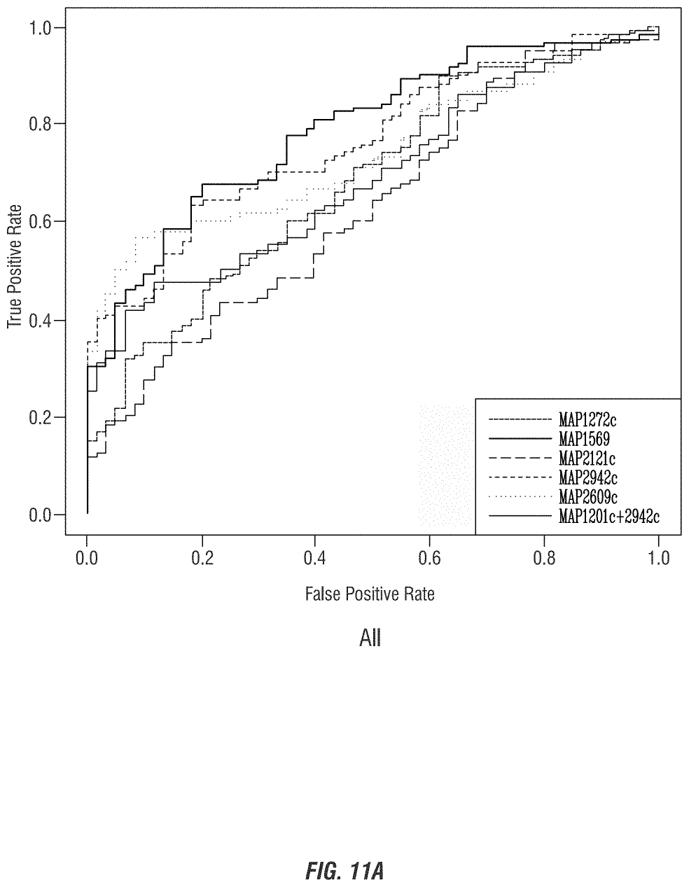

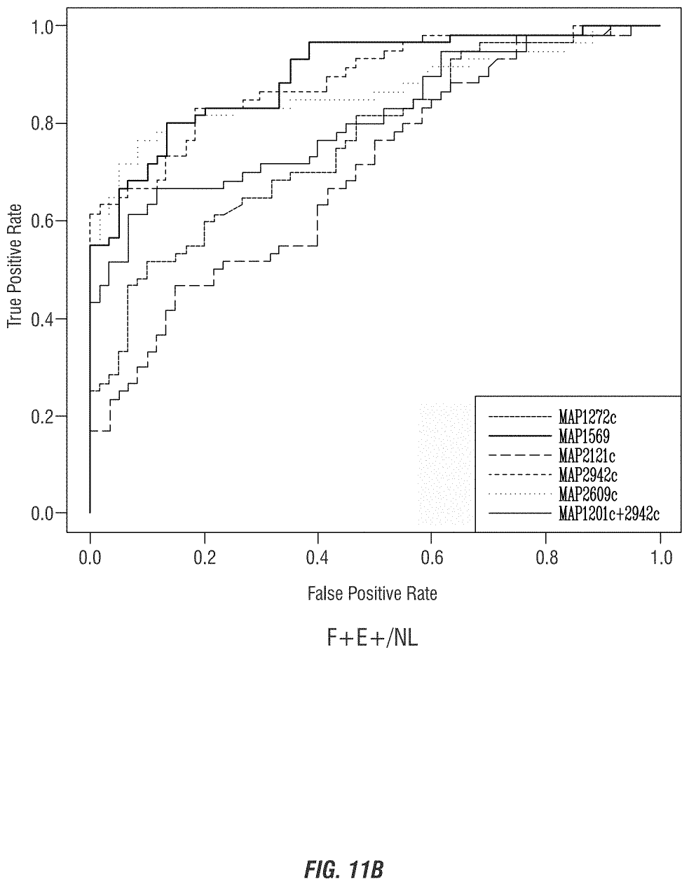

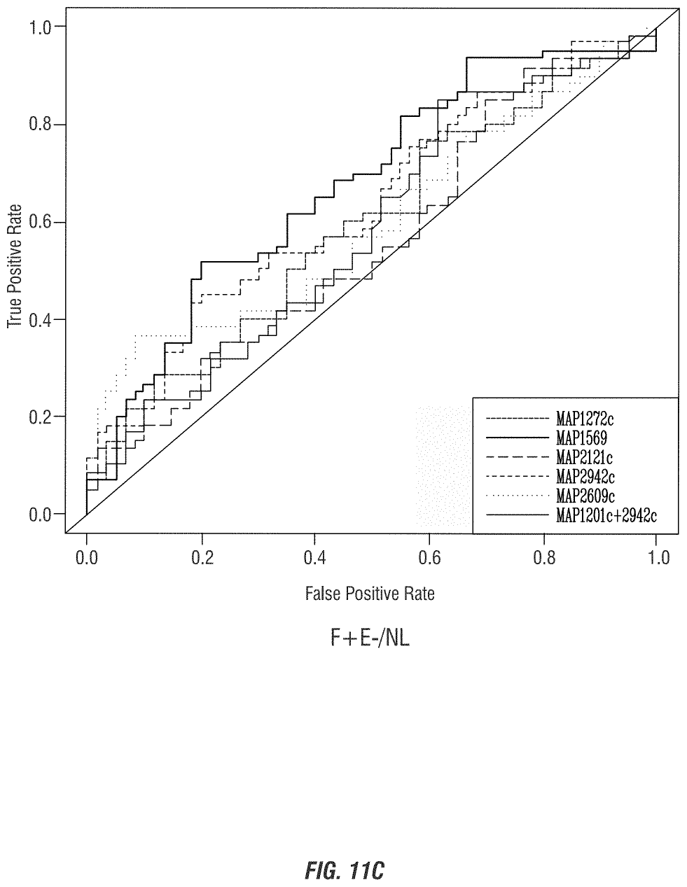

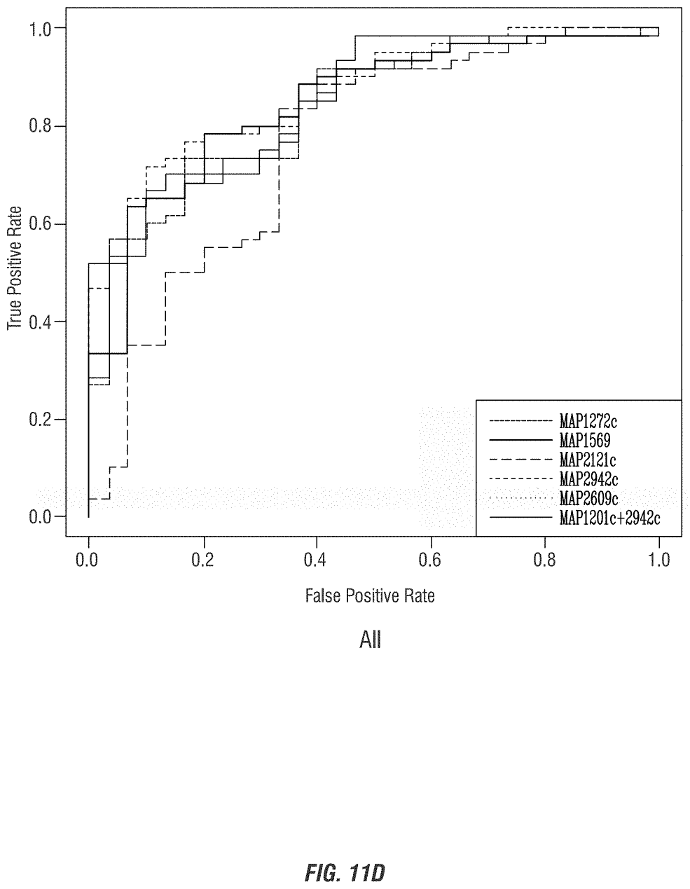

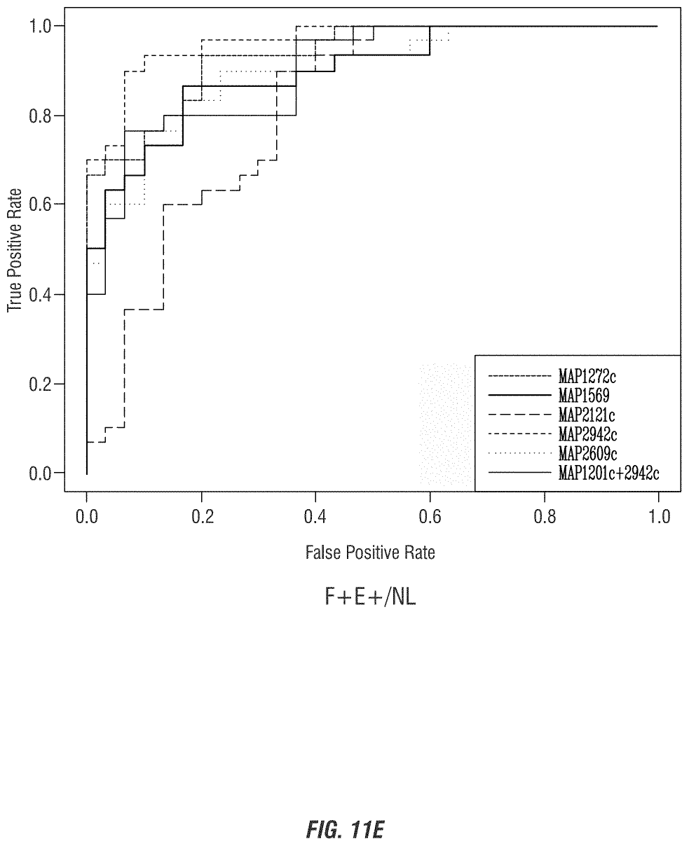

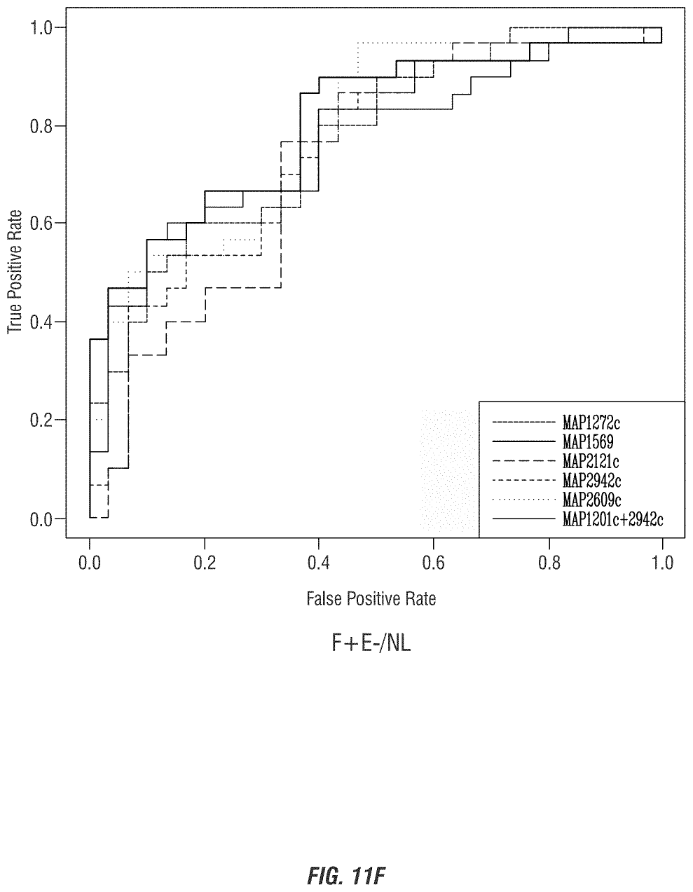

ROC analysis for the 6 antigens was performed with the 180 serum samples and the 90 milk samples (FIG. 11) and the area under curve (AUC), preliminary sensitivity and specificity were determined for each antigen individually as well as in combination based on cutoff values at maximum Youden Index (Table 3). The AUCs for serum in all samples for each antigen ranged from 0.63 (MAP2121c) to 0.79 (MAP1569) with median 0.71. The AUCs for milk generally were higher than the corresponding values for serum and ranged from 0.77 (MAP2121c) to 0.87 (MAP1201c+2942c) with a median 0.828 (Table 3). We also calculated ROC curves for each of the F+E+, F+E−, and Overall (F+E+ and F+E−) groups individually (Table 3), and AUCs ranged from 0.70 (MAP2121c) to 0.90 (MAP1569) with median 0.839 in serum in the F+E+ group, and 0.81 (MAP2121c) to 0.97 (MAP2942c) in milk. As expected, these were lower for the F+E− group, ranging from 0.56 (MAP2121c) to 0.68 (MAP1569) in serum and 0.723 (MAP2121c) to 0.811 (MAP1201c+2942c) in milk.

TABLE 3
ROC analysis of MAP recombinant proteins

| | AUC | | | |
|---|---|---|---|---|
| | | | | Overall (F+ E+ |
| Antigen | | F+ E+ | F+ E− | and F+ E−) |
| MAP1272c | Serum | 0.7667 | 0.5994 | 0.6831 |
| | Milk | 0.8406 | 0.8011 | 0.8011 |
| MAP1569 | Serum | 0.9001 | 0.6768 | 0.7884 |
| | Milk | 0.8944 | 0.7456 | 0.8200 |
| MAP2121c | Serum | 0.6974 | 0.5567 | 0.6270 |
| | Milk | 0.8122 | 0.7233 | 0.7678 |
| MAP2942c | Serum | 0.8911 | 0.6322 | 0.7617 |
| | Milk | 0.9656 | 0.7711 | 0.8683 |
| MAP2609 | Serum | 0.8704 | 0.6061 | 0.7383 |
| | Milk | 0.9189 | 0.7522 | 0.8356 |
| MAP1201c + | Serum | 0.8083 | 0.5645 | 0.6865 |
| 2942c | Milk | 0.9367 | 0.8111 | 0.8739 |

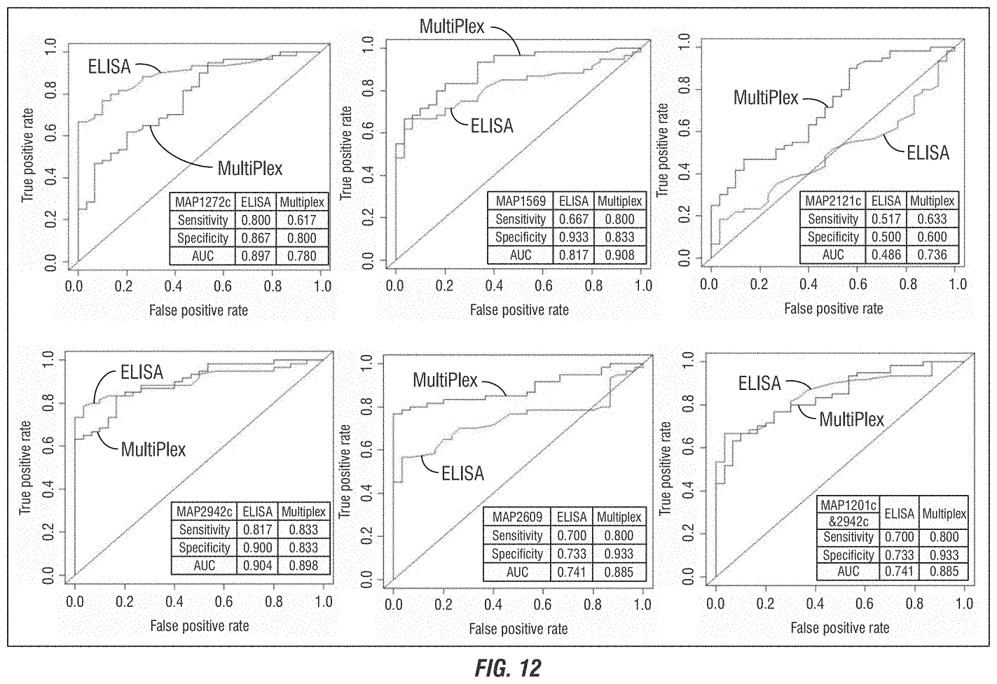

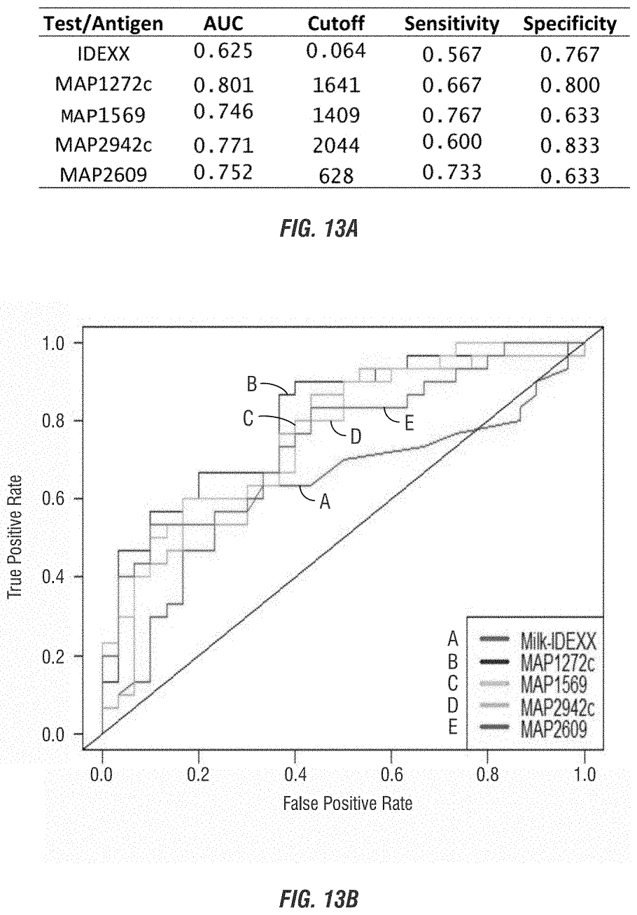

Next, we compared the ROC curves of serum samples generated from the multiplex assays with those from the ELISA using the same recombinant MAP antigens and noted higher multiplex AUCs in MAP1569, MAP2121c and MAP2942c and similar AUCs in the other three proteins (FIG. 12). This suggests that the multiplex test has higher sensitivity and specificity compared to using the same antigens in regular ELISA tests. We also compared ROC curves of milk multiplex results with those of milk ELISA using commercial IDEXX kits in the F+E− group. Multiplex AUCs of recombinant proteins were all higher compared to that obtained using IDEXX kits (FIG. 13), indicating an advantage of the multiplex assay in detection of early infection compared to commercial ELISA kits (FIG. 13).

### Concordance Between Serum and Milk Assays to Individual MAP Antigens

The agreement of serum and milk antibody reactivity was analyzed using the Spearman rank correlation and concordance correlation. The Spearman covariance R value ranged from 0.572 (MAP2121c) to 0.756 (MAP2942c) with median 0.661 (Table 4). The correlation between serum and milk for all antigens was significant (p<0.01). The concordance correlation coefficient (CCC) ranged from a relatively poor 0.55 (MAP2121c) to a moderate 0.79 (MAP2942c) with median CCC of 0.69 (Table 4). As noted earlier, the highest levels of precision and accuracy for both serum and milk were observed for MAP2942c.

TABLE 4
Concordance correlation between MFI
values from serum and milk assays

| | Spearman correlation | | Concordance | | |
|---|---|---|---|---|---|
| Antigens | covariance R | p value | CCC | Precision | Accuracy |
| MAP1272c | 0.587 | <0.01 | 0.6183 | 0.6475 | 0.9549 |
| MAP1569 | 0.673 | <0.01 | 0.6174 | 0.7049 | 0.8758 |
| MAP2121c | 0.572 | <0.01 | 0.5529 | 0.6115 | 0.9041 |
| MAP2942c | 0.756 | <0.01 | 0.7947 | 0.8104 | 0.9807 |
| MAP2609 | 0.649 | <0.01 | 0.6839 | 0.6926 | 0.9874 |
| MAP1201c + | 0.726 | <0.01 | 0.6971 | 0.7353 | 0.948 |
| 2942c | | | | | |
| 6 Ags | 0.677 | <0.01 | 0.6917 | 0.7207 | 0.9598 |
| combined | | | | | |
| 4 Ags | 0.668 | <0.01 | 0.6923 | 0.7172 | 0.9653 |
| combined | | | | | |

### Increased Sensitivity by Using a Combination of Recombinant Antigens

With the caveat that these are preliminary studies with a selected group of samples that preclude robust estimates of sensitivity and specificity, we noted from the ROC curves, the sensitivity of a single antigen assay was low, especially for the F+E− group. Therefore we tested whether using a combination of antigens increases the sensitivity. With the ROC cutoff (at maximum Youden Index), we calculated the sensitivity with a combination of all 6 antigens and the 4 most reactive antigens. In serum samples from the F+E+ group, the assay sensitivity increased from 0.63-0.81 using single antigens to 0.95 and 0.97 with 4- and 6-combined antigens, respectively. However, the assay specificity was reduced to 0.70 and 0.53 with 4- and 6-combined antigens. The four-antigen combination increased the specificity without obvious loss of sensitivity as compared to the combination of 6 antigens. To explore alternative approaches to increase assay specificity, we applied a cut-off using the mean+2SD of the NL, and re-estimated the sensitivity and specificity each antigen individually and in combination (Table 5). This increased (for the 4-antigen combination) predicted specificities of the assay in serum and milk to 0.87 and 0.90, respectively, and the sensitivity increased to 0.90 for serum and 0.93 for milk in the F+E+ group. As expected, although higher than single antigen (0.1-0.217 in serum, 0.27-0.47 in milk), the sensitivity of the combined 4-antigen assay is still lower in the F+E− group with 0.38 in serum and 0.57 in milk.

TABLE 5
Sensitivity and Specificity for serum and milk at M + 2SD cutoff

| | | | | | | MAP1201c + | *4- | **6- |
|---|---|---|---|---|---|---|---|---|
| Sample Type | MAP1272c | MAP1569 | MAP2121c | MAP2942c | MAP2609 | 2942c | combined | combined |
| Serum (n = 180) | | | | | | | | |
| Specificity | 0.950 | 0.950 | 0.967 | 0.950 | 0.983 | 0.933 | 0.867 | 0.783 |
| Overall Sensitivity | 0.200 | 0 408 | 0.183 | 0.408 | 0.417 | 0.350 | 0.642 | 0.675 |
| Sensitivity_F+E− | 0.100 | 0.167 | 0.133 | 0.183 | 0.217 | 0.150 | 0.383 | 0.433 |
| Sensitivity_F+E+ | 0.300 | 0.650 | 0.233 | 0.633 | 0.617 | 0.550 | 0.900 | 0.917 |
| Milk (n = 90) | | | | | | | | |
| Specificity | 0.967 | 0.933 | 0.933 | 0.933 | 0.967 | 0.933 | 0.900 | 0.867 |
| Overall Sensitivity | 0.517 | 0.417 | 0.300 | 0.583 | 0.467 | 0.583 | 0.750 | 0.783 |
| F+E− Sensitivity | 0.433 | 0.267 | 0.300 | 0.367 | 0.333 | 0.467 | 0.567 | 0.633 |
| F+E+ Sensitivity | 0.600 | 0.567 | 0.300 | 0.800 | 0.600 | 0.700 | 0.933 | 0.933 |

*4-combined: combination of MAP1272c, MAP1569, MAP2942c, and MAP2609
**6-combined: combination of all 6 antigens

### Discussion

Fluorescent bead-based multiplex assays have been rapidly gaining popularity for use in clinical microbiology and diagnostic laboratories due to their enhanced sensitivity and greater dynamic quantification range [27]. Despite these advantages, bead-based multiplex assays have not been tested for clinical diagnostic use in Johne's disease in animals. The results of our investigation demonstrate the feasibility of developing sensitive and specific immunoassays for the simultaneous detection of antibodies to selected MAP recombinant proteins in serum and milk samples from infected cows, especially during early infection in animals that are fecal test positive but negative with traditional commercial ELISA kits.

The results show that when used in combinations of up to 4 recombinant MAP antigens, more than 90% of infected cows in the F+E+ group were recognized (90% with serum and 93.3% with milk) with a specificity of 0.867 and 0.900. In the F+E− group in which all animals tested negative with two independent serum and one milk ELISA test kits, 38.3% and 56.7% of infected animals were successfully identified in serum and milk respectively, suggesting a higher sensitivity of the multiplex assay format for detection of cows during early stages of infection compared to all currently available ELISA tests. Importantly, with the exception of MAP1272c, the serum multiplex bead-based assays consistently showed higher sensitivity and specificity than the corresponding values for the ELISA (FIG. 12). We acknowledge that the sample set in this study is small and selected and that a robust determination of sensitivity and specificity must be based on a large collection of unbiased field samples in our future studies. Nevertheless, the existing data support the advantages of recombinant MAP antigen-based multiplex testing for improving specificity and sensitivity of serological Johne's assays and also suggest the feasibility of a multiplex Johne's assay for identifying infection in many animal before it can be reliably detected by current commercial ELISA kits.

Commercial milk ELISAs based whole MAP antigen preparations are commonly used for diagnosis of MAP infection in dairy cows. Antibody reactivity to individual MAP proteins in milk has not been evaluated in previous studies. This study demonstrated that individual MAP proteins are recognized by antibodies in milk samples during early MAP infection. Moreover, the milk assay using the same MAP antigens showed even higher sensitivity and specificity than the respective serum assay. Compared to the NL group, elevated amounts of antibodies were seen in the F+E− group with all 6 recombinant MAP proteins (p<0.05) in milk while only 3 recombinant proteins were recognized using sera. This suggests that multiplex assays could be easily adapted to the milk sampling format and demonstrates that the antigens are adequate for the purposes of the invention, although further validation in a larger number of milk samples needs to be performed in future studies. In contrast to human milk, where IgA is the dominant antibody class, IgG is typically greater than 75% of total immunoglobulin content in cow (or goat, sheep) colostrum and milk [28,29]. Therefore, in the current multiplex immunoassays in milk, most of the reactivity can likely be associated with IgG.

Previous studies investigating factors that influence the outcome of MAP ELISA in milk have suggested the role of a number of factors including milk yield (concentration of MAP-specific antibodies, mainly related to days in milk, DIM), herd (prevalence of JD), and parity (related to number of lactation) were mainly attributed [30,31]. In our investigation, days in milk (DIM) and lactation numbers were considered for animals in each group, and the results show no significant difference for DIM and lactation number between groups (Table 1). Considering that milk from a cow is easily obtained in a non-invasive manner with lower cost compared with the collection of serum, our studies suggest that it may be feasible to develop milk-based rapid and sensitive multiplex assays for the early detection of MAP infection in dairy animals.

Of the six candidate antigens tested in the multiplex assays, three antigens (MAP1569, MAP2942c, and MAP2609) showed significantly increased MFIs on group comparison and higher AUC on their ROC curves in the F+E− group, indicating higher sensitivity for detecting antibody responses in cows with early-infection. MAP1569, a secreted protein, was also identified from MAP culture filtrates and previously shown to be recognized by sera from MAP-infected cows [32]. The recombinant MAP1569 (ModD) protein was evaluated as an antigen with serum samples from infected and control cattle (infected n=444, control n=412) by ELISA, and ROC analysis showed AUC 0.533 in cows that were fecal culture-positive for MAP and control negative cows [16]. This is significantly lower than the AUC 0.788 in all serum samples with multiplex assay in this study, and even lower than AUC 0.677 in the F+E− group (Table 3). Similarly, secreted proteins MAP2942c and MAP2609, were also investigated in previous studies and shown to be recognized by sera from infected cows, though only a small number of sera (n=11) were tested [33]. The other 3 candidate antigens evaluated in this study (MAP1272c, MAP2121c, and MAP1201c+2942c) were not able to detect infection in the F+E− group with serum assay, but were able to detect infection in the milk assay. Although the response to MAP1272c was not significantly higher in F+E− than in the control (NL), its addition to the combination of antigens increased the sensitivity. MAP2121c in both serum and milk ROC analysis showed the lowest specificity (serum 0.583 and milk 0.667), suggesting it may not be a good candidate for use in an immunodiagnostic setting. Curiously, the results suggest that the fusion protein MAP1201c+2942c did not exhibit increased antibody reactivity as compared with MAP2942c alone. Additionally, higher background was seen in this fusion protein compared to MAP2942c alone, suggesting that careful attention will need to be paid for reducing specificity when using fusion proteins for assays of this nature, particularly since it is relatively easy to include or exclude specific antigens to increase sensitivity or discriminatory power using the bead-based multiplex assays.

The studies show that despite the fact that the new multiplex assays are more sensitive than the existing ones in the F+E− group and have proven adequate for the purposes of the invention, the specificity and sensitivity values still need further improvement for reliable early serological diagnostic of Johne's disease. While there are many potential biological factors that could contribute to this finding, we note that one simple explanation for the low specificity values may also be that cows that are actually exposed and infected were not recognized as such with the existing low sensitivity assays, and hence treated as “negative” when they were actually “positive”, considering several studies have previously reported that MAP was recovered from tissues of cattle during slaughter despite negative fecal culture or PCR tests and being from “low” prevalence herds [34-36]. We carefully analyzed the cows in the NL group considered as the “true negatives” in our study. These cows were all from two herds, 33 were from herd A (herd size 222) and 27 from herd G (size 287), and both herds were categorized as uninfected based on a prevalence (rate 0%) with ELISA tests one year before sample collection. Samples, including serum, milk, and feces, were collected from 136 cows in herd A and 175 cows in herd G, and examined with serum and milk ELISAs, fecal cultures, and fecal PCRs. If a cow with any one positive of the 8 tests is considered as infected, there were 10 from herd A and 5 from herd G, which indicates infected cows possibly existed in these two “uninfected” herds, and the results of the specificity and sensitivity analyses have to be considered in this light.

An additional source of non-specific reactivity may have resulted from the inclusion of MBP as part of the MAP fusion protein to facilitate proper folding and solubilization of the expressed proteins [24,37]. Since MBP has previously been shown to be recognized by sera from a small number of cattle and sheep, and antigenicity after cleavage and removal of MBP has been shown to be marginally enhanced [24,38], future studies may need to consider the inclusion of controls with beads-coupled with MBP or use recombinant proteins without the MBP tag [38] to help reduce non-specific binding. Finally, taken together in context of the fact that the candidate proteins evaluated in this study represented only a small subset of those that were found to be immunogenic using sera from our previous MTB and MAP protein array studies [17; Example 1], it is quite likely that the screening of additional recombinant MAP proteins in future studies. Although the MAP antigens disclosed herein have proven adequate for the purposes of the invention, antigens that are able to better discriminate the F+E− group, may provide considerable potential to further enhance the sensitivity and specificity of the multiplex assay for detection of MAP infected animals during the early stages of infection and thereby help with disease control efforts.

### REFERENCES

1. Cocito C, Gilot P, Coene M, de Kesel M, Poupart P, Vannuffel P (1994) Paratuberculosis. Clin Microbiol Rev 7: 328-345.
2. Clarke C J (1997) The pathology and pathogenesis of paratuberculosis in ruminants and other species. J Comp Pathol 116: 217-261.
3. Stewart D J, Vaughan J A, Stiles P L, Noske P J, Tizard M L, Prowse S J, et al. (2004) A long-term study in Merino sheep experimentally infected with Mycobacterium avium subsp. paratuberculosis: clinical disease, faecal culture and immunological studies. Vet Microbiol 104: 165-178.
4. Ott S L, Wells S J, Wagner B A (1999) Herd-level economic losses associated with Johne's disease on U S dairy operations. Prev Vet Med 40: 179-192.
5. Collins M T, Wells S J, Petrini K R, Collins J E, Schultz R D, Whitlock R H (2005) Evaluation of five antibody detection tests for diagnosis of bovine paratuberculosis. Clin Diagn Lab Immunol 12: 685-692.
6. Sweeney R W, Whitlock R H, McAdams S, Fyock T (2006) Longitudinal study of ELISA seroreactivity to Mycobacterium avium subsp. paratuberculosis in infected cattle and culture-negative herd mates. J Vet Diagn Invest 18: 2-6.
7. Yokomizo Y, Yugi H, Merkal R S (1985) A method for avoiding false-positive reactions in an enzyme-linked immunosorbent assay (ELISA) for the diagnosis of bovine paratuberculosis. Nihon Juigaku Zasshi 47: 111-119.
8. McKenna S L, Keefe G P, Barkema H W, Sockett D C (2005) Evaluation of three ELISAs for Mycobacterium avium subsp. paratuberculosis using tissue and fecal culture as comparison standards. Vet Microbiol 110: 105-111.
9. Li L, Bannantine J P, Zhang Q, Amonsin A, May B J, Alt D, et al. (2005) The complete genome sequence of Mycobacterium avium subspecies paratuberculosis. Proc Natl Acad Sci USA 102: 12344-12349.
10. Shin S J, Yoo H S, McDonough S P, Chang Y F (2004) Comparative antibody response of five recombinant antigens in related to bacterial shedding levels and development of serological diagnosis based on 35 kDa antigen for Mycobacterium avium subsp. paratuberculosis. J Vet Sci 5: 111-117.
11. Leroy B, Roupie V, Noel-Georis 1, Rosseels V, Walravens K, Govaerts M, et al. (2007) Antigen discovery: a postgenomic approach to paratuberculosis diagnosis. Proteomics 7: 1164-1176.
12. Gumber S, Taylor D L, Whittington R J (2009) Evaluation of the immunogenicity of recombinant stress-associated proteins during Mycobacterium avium subsp. paratuberculosis infection: Implications for pathogenesis and diagnosis. Vet Microbiol 137: 290-296.
13. Nagata R, Kawaji S, Mori Y (2013) Use of enoyl coenzyme A hydratase of Mycobacterium avium subsp. paratuberculosis for the serological diagnosis of Johne's disease. Vet Immunol Immunopathol 155: 253-258.
14. Waters W R, Miller J M, Palmer M V, Stabel J R, Jones D E, Koistinen K A, et al. (2003) Early induction of humoral and cellular immune responses during experimental Mycobacterium avium subsp paratuberculosis infection of calves. Infection and Immunity 71: 5130-5138.
15. Paustian M L, Amonsin A, Kapur V, Bannantine J P (2004) Characterization of novel coding sequences specific to Mycobacterium avium subsp. paratuberculosis: implications for diagnosis of Johne's Disease. J Clin Microbiol 42: 2675-2681.
16. Cho D, Shin S J, Talaat A M, Collins M T (2007) Cloning, expression, purification and serodiagnostic evaluation of fourteen Mycobacterium paratuberculosis proteins. Protein Expr Purif 53: 411-420.
17. Li L, Bannantine J P, Campo J J, Randall A, Grohn Y T, Katani R, et al. (2017) Identification of sero-reactive antigens for the early diagnosis of Johne's disease in cattle. PLoS One 12: e0184373.
18. Wagner B, Freer H (2009) Development of a bead-based multiplex assay for simultaneous quantification of cytokines in horses. Vet Immunol Immunopathol 127: 242-248.
19. Wagner B, Freer H, Rollins A, Erb H N, Lu Z, Grohn Y (2011) Development of a multiplex assay for the detection of antibodies to Borrelia burgdorferi in horses and its validation using Bayesian and conventional statistical methods. Vet Immunol Immunopathol 144:374-381.
20. Baud D, Zufferey J, Hohlfeld P, Greub G (2014) Performance of an automated multiplex immunofluorescence assay for detection of Chlamydia trachomatis immunoglobulin G. Diagn Microbiol Infect Dis 78: 217-219.
21. Andrade D C, Borges I C, Laitinen H, Ekstrom N, Adrian P V, Meinke A, et al. (2014) A fluorescent multiplexed bead-based immunoassay (FMIA) for quantitation of IgG against Streptococcus pneumoniae, Haemophilus influenzae and Moraxella catarrhalis protein antigens. J Immunol Methods 405: 130-143.
22. Wagner B, Goodman L B, Babasyan S, Freer H, Torsteinsdottir S, Svansson V, et al. (2015) Antibody and cellular immune responses of naive mares to repeated vaccination with an inactivated equine herpesvirus vaccine. Vaccine 33: 5588-5597.
23. Leite F L, Reinhardt T A, Bannantine J P, Stabel J R (2015) Envelope protein complexes of Mycobacterium avium subsp. paratuberculosis and their antigenicity. Vet Microbiol 175: 275-285.
24. Bannantine J P, Hansen J K, Paustian M L, Amonsin A, Li L L, Stabel J R, et al. (2004) Expression and immunogenicity of proteins encoded by sequences specific to Mycobacterium avium subsp. paratuberculosis. J Clin Microbiol 42: 106-114.
25. Bannantine J P, Campo J J, Li L, Randall A, Pablo J, Praul C A, et al. (2017) Identification of Novel Seroreactive Antigens in Johne's Disease Cattle Using the Mycobacterium tuberculosis Protein Array. Clin Vaccine Immunol.
26. Greiner M, Pfeiffer D, Smith R D (2000) Principles and practical application of the receiver-operating characteristic analysis for diagnostic tests. Prev Vet Med 45: 23-41.
27. Christopher-Hennings J, Araujo K P, Souza C J, Fang Y, Lawson S, Nelson E A, et al. (2013) Opportunities for bead-based multiplex assays in veterinary diagnostic laboratories. J Vet Diagn Invest 25: 671-691.
28. Gapper L W, Copestake D E, Otter D E, Indyk H E (2007) Analysis of bovine immunoglobulin G in milk, colostrum and dietary supplements: a review. Anal Bioanal Chem 389: 93-109.
29. Hurley W L, Theil P K (2011) Perspectives on immunoglobulins in colostrum and milk. Nutrients 3: 442-474.
30. Nielsen S S, Grohn Y T, Enevoldsen C (2002) Variation of the milk antibody response to paratuberculosis in naturally infected dairy cows. J Dairy Sci 85: 2795-2802.
31. Eisenberg S W, Veldman E, Rutten V P, Koets A P (2015) A longitudinal study of factors influencing the result of a Mycobacterium avium ssp. paratuberculosis antibody ELISA in milk of dairy cows. J Dairy Sci 98: 2345-2355.
32. Facciuolo A, Kelton D F, Mutharia L M (2013) Novel secreted antigens of Mycobacterium paratuberculosis as serodiagnostic biomarkers for Johne's disease in cattle. Clin Vaccine Immunol 20: 1783-1791.
33. Willemsen P T, Westerveen J, Dinkla A, Bakker D, van Zijderveld F G, Thole J E (2006) Secreted antigens of Mycobacterium avium subspecies paratuberculosis as prominent immune targets. Vet Microbiol 114: 337-344.
34. Whitlock R H, Wells S J, Sweeney R W, Van Tiem J (2000) ELISA and fecal culture for paratuberculosis (Johne's disease): sensitivity and specificity of each method. Vet Microbiol 77: 387-398.
35. Pribylova R, Slana I, Kralik P, Kralova A, Babak V, Pavlik 1(2011) Correlation of Mycobacterium avium subsp. paratuberculosis counts in gastrointestinal tract, muscles of the diaphragm and the masseter of dairy cattle and potential risk for consumers. Int J Food Microbiol 151: 314-318.
36. Khol J L, Pinedo P J, Buergelt C D, Neumann L M, Rae D O (2014) Lymphatic fluid for the detection of Mycobacterium avium subsp. paratuberculosis in cows by PCR, compared to fecal sampling and detection of antibodies in blood and milk. Vet Microbiol 172: 301-308.
37. Sachdev D, Chirgwin J M (2000) Fusions to maltose-binding protein: control of folding and solubility in protein purification. Methods Enzymol 326: 312-321.
38. Gurung R B, Begg D J, Purdie A C, Bannantine J P, Whittington R J (2013) Antigenicity of Recombinant Maltose Binding Protein-Mycobacterium avium subsp paratuberculosis Fusion Proteins with and without Factor Xa Cleaving. Clinical and Vaccine Immunology 20: 1817-1826.

### Example 3

TABLE 6
Additional antigens identified in MAP protein microarray

| | Identified | | Predicted |
|---|---|---|---|
| | in | | Subcellular |
| MAP ID | group | Functional description | Localization |
| MAP0019c | F+E− only | penicillin-binding protein | Membrane |
| MAP0117 | NH only | hypothetical protein | Membrane |
| MAP0123 | NH only | hypothetical protein | Cytoplasmic |
| MAP0357 | NH_F+E− | conserved membrane protein | Membrane |
| MAP0433c | NH only | hypothetical protein | Membrane |
| MAP0616c | F+E− only | hypothetical protein | Membrane |
| MAP0646c | NH only | hypothetical protein | Membrane |
| MAP0858 | NH_F+E− | hypothetical protein (MAP unique) | Cytoplasmic |
| MAP0953 | NH_F+E−_F+E+ | hypothetical protein | Membrane |
| MAP1152 | F+E−_F+E+ | PPE-family protein | Cytoplasmic |
| MAP1224c | F+E−_F+E+ | conserved membrane protein | Membrane |
| MAP1298 | F+E− only | inositol-monophosphatase | Cytoplasmic |
| MAP1506 | F+E−_F+E+ | PPE family protein | Cytoplasmic |
| MAP1525 | NH only | hypothetical protein | Membrane |
| MAP1561C | NH_F+E− | probable NADH dehydrogenase | Cytoplasmic |
| MAP1651c | F+E+ only | hypothetical protein | Cytoplasmic |
| MAP1761c | NH only | hypothetical protein | Extracellular |
| MAP1782c | F+E− only | cytochrome P450 | Cytoplasmic |
| MAP1960 | F+E−_F+E+ | hypothetical protein | Membrane |
| MAP1968c | NH_F+E− | hypothetical protein | Extracellular |
| MAP1986 | NH_F+E−_F+E+ | conserved transmembrane protein | Membrane |
| MAP2093C | NH only | arginine/ornithine transporterv RocE | Membrane |
| MAP2100 | NH only | ABC transporter | Membrane |
| MAP2117c | F+E− only | conserved integral membrane protein | Membrane |
| MAP2158 | F+E− only | hypothetical protein (MAP unique) | Cytoplasmic |
| MAP2187C | NH_F+E− F+E+ | hypothetical protein | Cytoplasmic |
| MAP2195 | F+E−F+E+ | hypothetical protein | Cytoplasmic |
| MAP2288c | NH only | hypothetical protein | Cytoplasmic |
| MAP2447c | NH_F+E−_F+E+ | hypothetical protein | Cytoplasmic |
| MAP2497c | NH_F+E−_F+E+ | lipoprotein | Extracellular |
| MAP2694 | NH only | hypothetical protein | Cytoplasmic |
| MAP2875 | F+E−_F+E+ | hypothetical protein | Cytoplasmic |
| MAP3039c | F+E−_F+E+ | hypothetical protein | Cytoplasmic |
| MAP3305c | NH_F+E−_F+E+ | Inpolhetical protein | Cytoplasmic |
| MAP3527 | NH only | Probable serine protease PepA | Membrane |
| MAP3531c | F+E+ only | secreted fibronectin-binding protein C | Membrane |
| MAP3540c | F+E−_F+E+ | hypothetical protein | Cytoplasmic |
| MAP3762c | F+E− only | glycosyltransferase | Cytoplasmic |
| MAP3773c | NH only | hypothetical protein | Cytoplasmic |
| AUP3852c | F+E−_F+E+ | hypothetical protein | Cytoplasmic |
| MAP4074 | F+E−_F+E+ | hypothetical protein | Cytoplasmic |
| MAP4143 | NH only | elongation factor Tu | Cytoplasmic |
| MAP4225c | NH_F+E−_F+E+ | dTDP-glucose 4,6-dehydratase | Cytoplasmic |
| MAP4231 | F+E− only | 30S ribosomal protein | Cytoplasmic |
| MAP4339 | NH only | hypothetical protein | Cytoplasmic |

>MAP0019c (SEQ ID NO: 1) pbpA-4808476: 4809954 MW: 51806.145
MNASLRRISVTVMALIVLLLLNATMTQVFAADSLRADPRNQRVLLDEYSRQRGQIVAGGQ
LLAYSVATDNRFRFLRVYPNPAQYAPVTGFYSLRYSSTGLERAEDPLLNGSDERLFGRRL
ADFFTGRDPRGANVDTTIRPRVQQAAWDGMQQGCGGPPCKGAVVALEPSTGKILAMVSSP
SYDPNLLSSHDPEVQAQAWQRLRDDPDNPMTNRAISETYPPGSTFKVITTAAALQAGASD
TEQLTAAPSIPLPNSTATLENYGGQACGNDPTVSLQQAFALSCNTAFVQLGILTGADALR
SMARSFGLDSTPSVIPLQVAESTIGIIPDAAALGMSSIGQKDVALTPLQNAEIAATIANG
GVTMQPYLVDSLKGPDLTTISTTTPYEQRRAVSPQVAAKLTELMVGAEKVAQQKGAIPGV
QIASKTGTAEHGSDPRHTPPHAWYIAFARAQTPKVAVAVLVENGADRLSATGGAIAAPIG
RAVIEAALQGGP
>MAP0047c (SEQ ID NO: 2)-4777324: 4778547 MW: 41082.03
LVSVALRTDQGFIPAVFRACSPPLTCSYSQPLSTASGRQPWEGPTQMIEIVPGHRALLGG
MVAGLIGLAVAAGGTASADPLPPAPAPVPAPAPANLGPELVPPSRYLAAPQATTAATQVT
PATPGTPGPAPAPAPAPAPAPAPATSGTIREFLQSKGVKFEAQKPQGFKALDITLPMPAR
WTQVPDPNVPDAFAVIADRHGSSIYSSNAQVVVYKLVGNFDPREAITHGYVDSQKLPAWQ
PTNASMADFGGFPSSIVEGTYRDGDLTLNTSRRHVIATSGPDKYLVSLAVTTDRAVAVAD
APATDAIVNGFRVTVPGASAPAPTAAPVALPAQAPAVAPVAPAPVAPAAPTAPAPAAAAP
LVPLAQTAPAAPAGLPAQPLPNQQHTPSLLAMVPGLPPLPNFSFLQH
>MAP0117 (SEQ ID NO: 3)-128027: 128251 MW: 8091.8164
MLSTIRKVLDYQLTIAELLGLGILLGTPYLIVGVIWSSTHTAHLHDMHGVDLWSFLGSI
VSWPVLLFANVCMT
>MAP0123 (SEQ ID NO: 4)-131103: 132008 MW: 30962.29
MTAPVWMALPPEVHSTLLSSGPGPGPLLAAAATWTGLSTQYDSAATELTAVLTGSMPVWD
GPTADRYVAAHMPYLAWLQLAGALSAEAAAQHQGVATAYTAALAAMPTLPELAAMPTLPE
LAANHATHAALVATNFFGVNTIPIAVNEADYARMWTQAATTMTTYQATTEAVQMSSVAGS
GTGGRPAAAAGPERERARGPERAPALGPEPAPVREPEVAPAVGPAPAAAAVRVPFSCPPQ
KRSGRCCSGPTVSRSPVRASRTGARRSTCRISGISSTATLRPWPGSSRTFRACSTRPSSRR
>MAP0210c (SEQ ID NO: 5) pirG-4613953: 4614963 MW: 30672.818
VPNRRRRKLSTAMSAVAALAVASPCAYFLVYESTAGNKAPEHHEFKQAAVMSDLPGELMG
ALSQGLSQFGINLPPVPALSGGATSTPGLASPGLGSPGLGTPGLGTPGLTNPGLTSPGAT
SPGLTSPGLTSPGLTSPGLTSPGAAPTTPGLTAPGALPTTPGGGVATPGAGLNPALSNPG
LTSPAGTAPGLGSPTVAPSEVPIDSGAGLDPGAGGTYPILGDPSTFGNASPIGGGGTGLG
GGSSSGGSGGLVNDVMQAANQLGAGQAIDLLKGLVMPAITQGMHGGAAAGALPGAAGALP
GAAGALPGAAGALPGAAGAAGALPAAAGAAPALPPV
>MAP0270 (SEQ ID NO: 6) fadE36-289434: 290486 MW: 38355.88
VTSADQLEGLDLAALDSYLRSLGIGRDGELRAEFISGGRSNLTFRVYDDATSWLVRRPPL
HGLTPSAHDMAREYRVVAALQDTPVPVARTIGLCEDESVLGAPFQIVEFVAGQVVRRRAQ
LESFSHTVIEGCVDSLIRVLVDLHSVDPDAVGLADFGKPSGYLERQVRRWGSQWALVRLP
EDRRDADVERLHSGLGQAIPQQSRTSIVHGDYRIDNTILDADDPTKVRAVVDWELSTLGD
PLSDAALMCVYRDPALDLIVNAQAAWTSPLLPTADSLADRYSLVAGIPLAHWEFYMALAY
FKLAIIAAGIDFRRRMSDQARGLGDAAEHTPEWAPLISRGLAELAKLPG
>MAP0353 (SEQ ID NO: 7) Converts glycerol and ADP to
glycerol-3-phosphatE−377404: 378951 MW: 55870.137
VSPNRRAVAEFAEFIAAIDQGTTSTRCMIFDHQGAEVARHQLEHEQILPRAGWVEHDPIE
IWERTSSVLTSVLNRANLSAENLAALGITNQRETTLVWNRKTGRPYYNAIVWQDTRTDRI
ASALDRDGRGQVIRRKAGLPPATYFSGAKLQWILDNVDGVREAAERGDALFGTADSWVLW
QLTGGPRGGVHATDVTNASRTMLMDLETLDWDDELLSFFTIPRAMLPEIGPSSSPRPFGV
TSDTGPAGGRIPITAVLGDQHAAMVGQVCLAEGEAKNTYGTGNFLLLNTGESIVRSEHGL
LTTVCYQFGDAKPVYALEGSIAVTGAAVQWLRDQLGIISGAAQSESLARQVDDNGGVYFV
PAFSGLFAPYWRSDARGAIVGLSRFNTNAHLARATLEAICYQSRDVVDAMAADSGVRLEV
LKVDGGITGNDLCMQIQADVLGVDWRPWAETTALGAAYAAGLAVGFWADPGELRANWR
EDKRWTPAWSDEQRTAGYAGWHKAVQRTLDWADVT
>MAP0356c (SEQ ID NO: 8)-4448623: 4449492 MW: 30473.078
MSEVVTGDAVVLDVQIAQLPVRALSALIDIAVIVVGYLLGLMLWAATLTQFDTALSNAIL
LIFTVLVIVGYPLILETATRGRSVGKIALGLRVVSDDGGPERFRQALFRALASLVEIWML
FGSPAVICSILSPKAKRIGDIFASTVVVNERGPRLGPPPAMPPSLAWWASSLQLSGLSSG
OAEVARQFLSRAAQLDPGLRLQMAYRIAGDVVARIAPPPPGAPPELVLAAVLAERHRREL
ARLRPPAPWPAPGYPPAWPGSGPAPQWPAPGPANPGPPEGFSAGFTPPR
>MAP0357 (SEQ ID NO: 9)-381240: 382232 MW: 34973.83
VDVDAFVLAHRPTWDRLDRLVGRRRSLSGAEIDELVELYQRVSTHLSMLRSASSDSMLVG
RLSSLVARARSAVTAAHAPLSSTFVRFWTVSFRVVAYRSWRWWVATGAAFFAVVVIVALW
VAGNPEVQSALGTPSDIDQLVNHDVESYYSEHPAAAFALQIWVNNSWVSAQCIALSVVLG
LPIPLVLFENAANLGVIAGLMFPAGKGGLLLGLLAPHGLLELTAVFLAGATGMRLGWSVI
SPGDRPRGQVLAEQGRAVVSVAVGLVAVLLVSGLIEALVTPSPLFTFVRVGIGVVAEAAF
LCYIGYFGRRGVKAGESGDIEEAPDWPAG
>MAP0394c (SEQ ID NO: 10)-4410101: 4411243 MW: 40815.445
MSTTPKQLDMAAILADTTNRVVVCCGAGGVGKTTTAAAIALRAAEYGRNVCVLTIDPAKR
LAQALGVNDLGNTPQRVPLAAEVPGELHAMMLDMRRTFDEMVVQYSGPGRAQAILDNQFY
QTVASSLAGTQEYMAMEKLGQLLAEDRWDLVVVDTPPSRNALDFLDAPKRLGSFMDSRLW
RLLLAPGRGIGRLVTGAMGLAMKAMSTILGSQMLADAAAFVQSLDATFGGFREKADRTYA
LLKRRGTQFVVVSAAEPDALREASFFVDRLSQEGMPLAGLVLNRTHPPLCSLPAERAIDG
TEMLEHDGDPETTSLAAAVLRIHADRAQTAKREIRLLSRFTGANPHVPVIGVPSLPFDVS
DLEALRALADQITSNQATAR
>MAP0433c (SEQ ID NO: 11)-4367917: 4369233 MW 44380.13
VGRLLFSNCGDTSGQRAESAAPMTEISASRGPVARGSMARVGTATAVTALCGYAVIYLAA
RDLAPGGFSVFGVFWGAFGLVTGAANGLLQETTREVRVMPYLEVAPVKRTHPLRVAMLLG
AAAAVVIAGSSPLWSGRVFVEARPLSVLLLSVGLAGFCVHATLLGMLAGTNEWTRYGALM
VTDAVIRVMVAAATVVLGWRLVGFLWATVAGAVAWLILLAASPATRATARLLTPGGTATF
LRGAAHSITAAGASAILVMGFPVLLKLTSAELGAQGGVIILAVTLTRAPLLVPLTAMQGN
LIAHFVDERSDRVRALIGPAAIVGAIGAVGVLAAGVLGPWVLRVVFGPQYQAGSALLAWL
TAAAVAIAMLTLTGAAAVAAALHRAYALGWVGATVASGLLLALPLSLQTRTVVGLLCGPL
VGIGVHLVALSRAARLTG
>MAP0523 (SEQ ID NO: 12) fadE28-549244: 550332 MW: 37458.664
MASETTMDFDPSPTQQAVADVVTSVLDRELSWEALVDGGVTALPVPERLGGDGVGLPEVA
TVLTEVGRRGAITPALATLGFAVLPLLELASEEQQDRFLAGVARGGVLTAALNEPGTPLP
DRPATTFADGRLSGTKIGVGYAAQADWMIVTADSAVVVVSPKADGVQVVQTPTSNGSDEY
TVSFTGVAVADSDVLAGATAARVNQLALAAVGAYADGLVSGALRLTADYVANRKQFGKPL
STFQTVAAQLAEVYIASRTIDLVAKSVVWGLSEGRDVDHDLGVLGYWVASQAPPAMQLCH
HLHGGMGMDITYPMHRYYSTIKDLTRLLGGPSHRLDLVAIASAAQPGAAGRHADDLVGAQ
CS
>MAP0568 (SEQ ID NO: 13) IprN-592824: 593978 MW: 41107.117
MSRMWLRAGGLATGSMLLAGCQFGGLNSLAMPGTAGHGSGAYSITVELPDVATLPQNSPV
MVDDVTVGSVAGISAEQRSDGSFYAAVKLALDKNVVLPANSTATVAQTSLLGSMHIDLNR
PKDRPAVGRLTDGSKIAEANTGRYPTTEEVLSALGVVVNKGNVGALEEITDETYRAVAGR
QDQFVDLVPRLAELTSGLNRQVNDIIDAVDGLNRFSASLARDKDNLGRALDTLPEAIRVL
NKNRDHIVEAFSALHKLADVTSHILAKTKVDFAADLKDLYAAVKALNDNRRNFVTSLQLL
LTFPFPNFGIKQAVRGDYLNVFTTFDLTLRRLGETFFTTAYFDPNMAHMNEILNPPDFLV
GEMANLSGQAADPFKIPPGTASGQ
>MAP0601c (SEQ ID NO: 14)-4203192: 4203839 MW: 23157.254
VSSDALVTITSDAGGETGQPPRNRRQEETFRKVLAAGIETLREKSYSDLTVRAVAARAKV
APATAYTYFSSKNHLIAEVYLDLVRQVPYFTDVNDPMPTRVEQVLRHLALVVADEPEVSA
ACTTALLSGGADPAVRAARDRIGVEIHRRITSAMGPDADPTTVSALEMSFFGALVQAGSG
EFSYREIADRLAYWRLILTGTTQASPETEAGDTR
>MAP0616c (SEQ ID NO: 15)-4187181: 4187627 MW: 15098.719
MAPLITLVVGSLVAWVVGRLGVAYVDGWAPALAVGLAAMFVLTGIAHFAPPLRADLVAIV
PPRLPAPGLLVSLTGVLELLGALGLLLPATRAAAAGCLLVLMLAMFPANIHASRMPDPPK
SMTTRLPLRIGMEIVFLAAAVAVALGGR
>MAP0646c (SEQ ID NO: 16)-4161040: 4161510 MW: 15856.8955
LPSSNTTTQPDLVDVRGPRFAAWVTTAVLVLALAVSAVSPAAAAVILAVQAVVFAIGAVG
GPRKHPYGRVFAAVVAPRLGPVREREPIPPLKFAQLVGLIFAVLGAAGFAAGASLFGLVA
TAAALAAAFLNAAFGICLGCQLYPLVARFRRPARST
>MAP0834c (SEQ ID NO. 17)-3977173: 3977874 MW: 25024.936
MDTAASSPRVLVVDDDSDVLASLERGLRLSGFEVSTAVDGAEALRSATETRPDAIVLDIN
MPVLDGVSVVTALRAMDNDVPVCVLSARSSVDDRVAGLEAGADDYLVKPFVLAELVARVK
ALLRRRGATATSSSETITVGPLEVDIPGRRARVNGVDVDLTKREFDLLAVLAEHKTAVLS
PAQLLELVWGYDFAADTNWDVFIGYLRRKLEANGGPRLLHTVRGVGFVLRMQ
>MAP0858 (SEQ ID NO: 18)-881207: 881755 MW: 19892.914
VRWTRRKPRSQTLTFAIEARCRECHYKATERAKVTTYPAERVADQLRPTPPAVPSKFGGL
WILAVVSASNSSTPAISPSAKCSRSAAVCQSSSTAPCIRLRSSRPSWSRADCSLAPLTSH
SAPGYRAVHDRSSYSAVCGTNAKALPVVRMKSSKFVLRSSVFAISCPLRHPCDLSELTRR
SR
>MAP0900 (SEQ ID NO: 19)-928761: 929657 MW: 29565.197
MTYSPGSPGYPPAQSGGTYAGATPSFAKDDDGKSKLPLYLNIAVVALGFAAYLLNFGPTF
TIGADLGPGIGGPAGDAGTAVVVALLAALLAGLGLLPKAKSYVGVVAVVAVLAALLAITE
TINLPAGFAIGWAMWPLVACVVLQAIAAVVVVLLDAGVITAPAPRPKYDPYAQYGQYGQY
GQYGQQPYYGQPGGQPGGQPGGQQHSPQGYGSQYGGYGQGGAPTGGFGAQPSPQSGPQQS
AQQQGPSTPPTGFPSFSPPPNVGGGSDSGSATANYSEQAGGQQSYGQEPSSPSGPTPA
>MAP0953 (SEQ ID NO: 20)-986288: 987778 MW: 52193.24
VTPIPYLRARHRLAVDGVLLAMFVFGCFVFGVLSVRRTTEGVLLTAALFCVVVYWVKPEG
MVGVTLFGAFAA1PEGLHVGKVFGPLTIYAYHLAAFLAICYLIPAAKPRSSDFLLPGILA
VTAVCSTVTGFLVGNSALVVTRESTTMLEMALGFVLALFVVYSGHVIWSIRVMIAILWFS
AGMAIVSSLYSIRLAGRAESLEGTTGAGQAMRIILSTQTPATAVLSALVAAPIVGRVRPR
LYLALGPPALSISLLSFSRNTLISMGVAAAVALLGSLSWAAVRRTIVAATVGATLVAVTV
PGSLFLLQRSKTGAWLADQYVAFSQRVLGGVTSSALAVDDSALERLREINLLKETIASAP
LFGHGLGYVYQPPTGDDEFHRYLYPAYSHNFYLWWLAKAGAVGMAAFVLFALTPVILALR
CTSGPAKIAAAVAAGLLAISAWPLPEMPMDALGLGMALGAAMGYAGLRRRERQLDDRCA
APGPTSNPVGVGTSS
>MAP0996c (SEQ ID NO: 21) kdpD-3791324: 3793900 MW: 92381.914
MMVDVTDVRDHHFKRGELRIYLGAAPGVGKTYSMLGEAHRRLERGTDLVAGVVETHGRAK
TAELLEGIEIIPPRYIEYRGGRFPELDVPAVLARHPQVVLVDELAHTKTPGSKNPKRWQD
VEELLDAGITVISTVNVQHLESLNDVVAQITGIEQKETVPDSVVRQASQVELIDITPEAL
RRRLSHGNVYAPDRIDAALSNYFRRGNLTALRELVLLWLADQVDTALAKYRAENKITDTW
EARERVVVAVTGGPESETLVRRASRIASKSSAELMVVHVIRGDGLAGLSESRMAKIRELA
SSLDASLHTIVGDEVPAALLEFAREMNATQLVIGTSRRSRWARLFEEGIGPRIVELSGKI
DVHLVTHEESKRGFRASSLAPRERRVASWLAALIVPSVICAVTVTWLDPYLDTGGESALF
FVGVLLVGLLGGIAPAALSAVLSGLLLNYYLIAPRHSFTIAEPNSAITELVLLLIAVAVA
VLVDFAAKRTREARRASQEAELLTLFAGSVLRGADLETLLERVRETYAQRSVSMLRESED
ARAGGTKTQVVACVGRDPCVSVDAADTAIEVGGPDSSEFQMLLAGRKLSARDRRVLSAVA
RQAAGLIRQRELAEEASRTEAIVRALELRRSLLSAVSHDLRTPLAAAKVAVSSLRAEDVA
FSPTDTAELLATIEESIDQLTALVGNLLDSSRLAAGAIHPDLRRVYLEEAVQRALVSIGK
GATGFFRSAIDRVKVDVGDAMVMADAGLLERVLANLIDNALRYAPNCVVRVNAGQVGDRV
LISVIDEGPGIPHGAEEQIFEAFQRLGDHDNTTGVGLGMSVARGFVEAMGGTITATDTPG
GGLTVMVDMAAPQSEGAA
>MAP1120 (SEQ ID NO: 22) OMP decarboxylase; OMPDCase;
OMPdecase; type 2 sub 1174477: 1175301 MW: 27487.605
VTGFGARLAAAKAQRGPLCVGIDPHPELLRAWDLPTTADGLAAFCDICVEAFAGFAVVKP
QVAFFEAYGAAGFAVLERTIAALRSAGVLVLADAKRGDIGTTMAAYAAAWAGDSPLAADA
VTASPYLGFGSLRPLLEAAAAHDRGVFVLAATSNPEGATVQRAAFDGRTVAQLVVDQAAV
VNRSTNPAGPGYVGVVVGATVLQPPDLSALGGPVLVPGLGVQGGRPEALAGLGGAEPGQL
LPAVAREVLRAGPDVAELRAAADRMLDAVAYLDA
>MAP1152 (SEQ ID NO: 23)-1207452: 1208702 MW: 40806.375
MDFGSLPPEINSGRIYSGPGSAPLLAAAAAWHGLAAEMHSAAASYGSAIAELRTLWHGPS
STAMAAAAAPFIAWLGGTAAQAEQTAAQATAAAAYDSVFAATVPPPVIAANPALLASLIA
TNVLGQNTPAIAATEAHYAEMWAQDAAAMYAYAGASAVATRLTPFGAPPQSADANAAADQ
SAAAASALQLSTASSVESALSOGVSQVPVAAQVNATAVTAAAQLPLSLTDITGILKTFNS
VMGTISGPYTPLGVANLAKNWYQIALSIPSVGTGIQGIGPLLHPKALTGVLAPLLRSDLL
TGSTALSSAGTVSASAGRAGLVGSLSVPANWASAVPAVRTVAAELPETMLDAAPAMAVNG
QQGMFGPTALSSLAGRAVGGTATRAVAGSTVRVPGAVAVDDLATTSTVIVIPPNAK
>MAP1201c (SEQ ID NO: 24) Catalyzes the conversion of
citrate to isocitratE−3566846: 3569659 MW: 101515.92
VTDSVNSFGARNTLKVGDKSYQIYRLDAVPNTEKLPYSLKVLAENLLRNEDGSNITKDHI
EAIANWDPKAEPSIEIQYTPARVVMQDFTGVPCIVDLATMREAIADLGGNPEKVNPLAPA
DLVIDHSVIADLFGTADTFERNVEIEYQRNGERYQFLRWGQGAFSDFKVVPPGTGIVHQV
NIEYLARVVMERDGVAYPDTCVGTDSKTTMVNGLGVLGWGVGGIEAEAAMLGQPVSMLIP
RVVGFKLTGEIQPGVTATDVVLTVTEMLRKHGVVGKFVEFYGEGVAEVPLANRATLGNMS
PEFGSTAAIFPIDEETIDYLKFTGRNAEQVALVETYAKEQGLWHDPAHEPAFSEYLELDL
SQVVPSIAGPKRPQDRIALSQAKSVFREQIPSYVGDGDGQQGYSKLDEVVDSTFPASDPG
APSNGHADDLPAVQSAAAHANGRPSNPVTVRSDELGEFVLDHGAVVITAVTSCTNTSNPE
VMLGAALLARNAVEKGLASKPWVKTTMAPGSQVVHDYYDKAGLWPYLEKLGFYLVGYGCT
TCIGNSGPLPEEISKAINDNDLSVTAVLSGNRNFEGRINPDVKMNYLASPPLVVAYALAG
TMDFDFEKQPLGKDKDGNDVYLKDIWPSQKDVSDTIASAINSEMFTKNYADVFKGDERWR
NLPTPSGNTFEWSPDSTYVRKPPYFEGMPAEPEPVADISGARVLALLGDSVTTDHISPAG
SIKPGTPAAQYLDEHGVDRKDYNSFGSRRGNHEVMIRGTFANIRLRNLLLDDVAGGYTRD
FTQDGGPQAFIYDAAQNYAAQNIPLVVLGGKEYGSGSSRDWAAKGTRLLGVRAVIAESFE
RIHRSNLIGMGVIPLQFPDGKSAKDLGLDGTEVFDITGIEELKKGKTPKTVHVKASKNGS
DAVEFDAVVRIDTPGEADYYRNGGILQYVLRNMLKSG
>MAP 1211 (SEQ ID NO: 25) protoheme ferro-lyase; catalyzes
the insertion of-1272041: 1273051 MW: 36321.484
MDFDAVLLLSFGGPEGPEQVRPFLENVTRGRGVPPERLDHVAEHYLHFGGVSPINGINRA
LIEQLRAAQDLPVYFGNRNWEPYVEDTVKVMRDNGIRRAAVFTTSAWSGYSSCTQYVEDI
ARARTAAGTGAPELVKLRPYFDHPLFVEMFAGAIADAAAKVPAGARLVFTAHSVPVAADE
RLGPRLYSRQVAYAARLVAAAAGYAEHDLVWQSRSGPPQVRWLEPDVADHLRALAESGTR
AVIVCPIGFVADHIEVVWDLDEELRAQAESAGMLMARASTPNAQPRFARLAADLIDELRC
GRTPARVTGPDPVPGCLASVNGAPCRPPHCAAQATG
>MAP1214 (SEQ ID NO: 26)-1274506: 1275639 MW: 40802.19
MQGAVAGLVLLAVLVIFAIVVVAKSVALIPQAEAAVIERLGRYSRTVSGQLTLLVPFIDR
IRARVDLRERVVSFPPQPVITEDNLTLNIDTVVYFQVTVPQAAVYEISNYIVGVEQLTTT
TLRNVVGGMTLEQTLTSRDQINGQLRGVLDEATGRWGLRVARVELRSIDPPPSIQASMEK
QMKADREKRAMILTAEGMRESATKEAEGQKQAQILAAEGAKQAAILAAEADRQSRMLRAQ
GERAAAYLQAQGQAKAIEKTFAAIKAGRPTPEMLAYQYLQTLPEMARGDANKVWVVPSDF
SAALQGFTKLLGTPGQDGVFRFQPSPVEDVPKHSADDDADVADWFSTETDPAIAQAVAKA
EAIARQPADGPTGELTQ
>MAP1215 (SEQ ID NO: 27)-1275658: 1276014 MW: 12292.815
MSALTSPKTYAALGVFHAVDAVACGVQVAPIRKTLDNLGVPDNIRPVLPVVKAAAAVGLL
SVTRFPGLARLTTAMLTLYFVLAVGAHVRVRDKVVNGLPAALFVALFAAMTVRGPERS
>MAP1224c (SEQ ID NO: 28)-3546401: 3547177 MW: 27161.354
LEGVTGSATSKIAETLRDLGCAIGAAARGVSRSRIAWTVAGITALVVLASLIPLPSPVQM
RDWAQSVGPWFPLAFLLAHIVVTVVPVPRTAFTLAAGLLFGPLLGVAIAVAASTASAMIA
MLLVRAAGWRLTRLVRHRSMDTVEERLRQRGWLAIVSLRLIPAVPFSALNYAAGASSVRV
LPYGLATLAGLLPGTAAVVILGDALAGHPSSLLYLVSALTSALGLTGLVIEIRHFRRHHR
RAHRHRDDEPSPEPATIG
>MAP1272c (SEQ ID NO: 29)-3469658: 3470608 MW: 33404.617
VRSQRGGPRPVHEPGRTREVTAPRPDECRRGQERPGKMKRIYAFAIGLALLGAPAAPMVV
PPVATADPGVRAMDYQQATDVVIARGLSQRGVPFSWAGGGINGPTRGTGTGANTVGFDAS
GLMQYAYAGAGIKLPRSSGAMYRVGQKILPQQARKGDLIFYGPEGTQSVAMYLGNNQMLE
VGDVVQVSPVRTAGMAPYMVRVLGTTAPTQQVPQQAPLQQTPAQQAPLQQTPGQQAPLQQ
TPGQQLPTQQAPLQQVPGQQVPGQQLPTQQAPQQAPLQLAPTQQAPLQQLPTQQSPLQQL
PVOQSPLQPAGAGLTR
>MAP1294 (SEQ ID NO: 30) catalyzes the formation of
L-histidinol phosphatE−1383574: 1384770 MW: 42180.035
VTGQRATPQPTLDDLPLRDDLRGKSPYGAPQLAVPVRLNTNENPHPPSRALVDDWRSVA
RAAADLHRYPDRDAVQLRSDLARYLTAQTGVQLGVENLWAANGSNEILQQLLQAFGGPGR
SAIGFVPSYSMHPIISDGTRTEWLQAARADDFSLDVDAAVAAVTERTPDVVFVASPNNPS
GQSVSLSGLRRLLDAAPGIVIVDEAYGEFSSQPSAVQLVGEYPTKLVVTRTMSKAFAFAG
GRLGYLIATPAVIEAMLLVRLPYHLSSVTQAAARAALRHADDTLGSVAALIAERERVSTA
LTGMGFRVIPSDANFVLFGEFTDAPASWQRYLDAGVLIRDVGIPGYLRATTGLAEENDAF
LRASAQLAATELAPVNVGAIANAAEPRAAGRDRVLGAP
>MAP1298 (SEQ ID NO: 31)impA-1386766: 1387566 MW: 27735.555
MDLDALVARASAILDDASKPFLAGHRADSAVRKKGNDFATDVDLAIERQVVAALVEATGI
GVHGEEFGGSAVDSEWVWVLDPVDGTFNYAAGSPMAGILLALLHHGDPVAGLTWLPFLDQ
RYTAVTGGPLRKNEIPRPPLTSIDLADALVGAGSFSADARGRFPGRYRMAVLENLSRVSS
RLRMHGSTGLDLAYVADGILGAAVSFGGHVWDHAAGVALVRAAGGVVTDLAGRPWTPASD
SALAAGPGAHAEILDILRNIGRPEDY
>MAP1501 (SEQ ID NO: 32)-1643809: 1645326 MW: 53338.29
VAEESRGQRGSGYGLGLSTRTQVTGYQFLARRTAMALTRWRVRMEVEPGRRQNLAVVASV
SAALVTCLGALLWSFISPAGQVGDSPIIADRDSGALYVRVGDRLYPALNLASARLITGRP
DNPHLVKSNQIASLPRGPMVGIPGAPSNFHPTGPSTSSWLVCDTVSNSTGAGAPSGVTVT
VIDAAPDLSNHRKVLTGSDAVVLNYGGDAWVIRDGRRSRIDATNRSVLLPLGLTPEQVSM
AKPMSRALYDALPVGPELTVPQIQNAGGAASFPGAPGPIGTVLVTPQISGPQQYSLVLAD
GVQTLPPLVAQILQNAGPGNTKPVTVEPSALAKMPVVNKLDLSSYPDAPLNVMDIRENPA
TCWWWQKTSGENRARVQVVSGATIPVAQKDVNKVVSLVKADTTGREADQVFFGPDYANFV
AVTGNDPGAKTTESLWWLTDAGARFGVDDTRDVREALGLKTKPSVAPWALRLLPQGPTL
SRADALVQHDTLPMDMSPAELAVPK
>MAP1506 (SEQ ID NO: 33)-1653138: 1654361 MW: 39695.39
MLDYGAFPPEFNSARIYSGPGSGSLVAAASAWSSLAAELNAAALSYDKVVTALASEEWLG
SASASMASAVAPYVGWMSTTAAQAEEAASQARAAAAAFEAALAASVPPPVIAANRMQVSQ
LQATNVLGQNTPLIAQFEAQYGEYWAQDAAAMYSYAGQSASASKVTPFQKAPQVTNPSGQ
VAQSAAVSTATANSTSTNTTKALQSLAQPASSSTTATKAATTAASTTSTDPLSEIWFLLT
GQTTLPTSLGSAVNGYSPFASLFYNTEGLPYFSTGMANTFTQIAKSVGAIGGAAPAAAKA
LPGLGGLGGMLGGGGAAAAHPVAALGGAGSIGGKLSVPVAWSGAPAAPALGHAIPVSSIS
AAPEAAGGPGNLLGGMPLAGAGAGGHGAAGPKYGFRPTVMARPPFAG
>MAP1525 (SEQ ID NO: 34)-1675815: 1676699 MW: 33837.46
LRDPVLVAIPFFLLLLTLEWTAARKLEHLTARPAPGAHQTRDSLTSISMGLVSVATTAGW
KTLALFGYAAIYAYLAPWHLPATRWYTWAIAILGVDLLYYAYHRIAHRVRLIWATHQAHH
SSEYYNFATALRQKWNNSGEILMWLPLPLLGIPPWMVFFSFSVNLIYQFWIHTERIDKLP
RPFEFVFNTPSHHRVHHGMDKVYLDKNYGGILIVWDRLFGTFQAELFRPHYGLTKHVDTF
NWTLQTRESVAIARDWRSASRLRDRLGYVFGPPGWAPRSAGRTAAGAPWTSL
>MAP1548c (SEQ ID NO: 35)-3129292: 3131343 MW: 70798.34
MGRHSAPDPDDFLDEPSPDHPVDERDDAYAFDAQGAPDEGYYPDERRYPDADFVADDDYA
PEEFAPGEDLVDEDPDDYPEFPSRRPATSGPQESPASAPSLRARRLDWRGGHRSEGGRRG
VSIGVIVALVAVVVVVGSVILWRFFGDALSKRSHTAAGRCVGGQEQVPVVADPSIADAIG
QFAESFNKSAGPIGDHCMVVSVKPAGSDAVLNGFIGKWPAELGGQPALWIPGSSVSAARL
AGATAQKTITESHSLASSPVVLAVRPELLPALSGQNWAALPGLQTNPNALAGLNLPAWGS
LRLALPMTGNGDAAFLAGEAVAAASVPPGAPVTQGTGAVRTLLSAQPKLADNSLTEAMNT
LLKPGDSASAPVHAVVTTEQQLFQRGQSLPDAKGALASWLPPGAAAVADYPTVLLSGSWL
TREQASAASEFSRFMHKSDQLAKLAKAGFRVNGGKPPSSPVTTFPALPSTLSVGDDAMRA
TLAEAMASPSTGQATTIMLDQSMPGQEGGKSRLANVIGALQDKIKALPASAVVGLWTFDG
HEGRSEVTSGPLADPVNGQPRSAALSAALDKQYSSSGGAVSFTTLRMIYQDMQSNYHAGQ
TNSILVITAGPHTDQTLDGPGLQDFIRKSADPAKPIAVNVIDFGADPDRTTWEAVAQLSG
GGYQNLATSASPDLATAVNAFLS
>MAP1553c (SEQ ID NO: 36) fadE14-3122199: 3123365 MW: 41352.83
LSAATTADIDHYRTVLAGAFDDQVLEWTREAEARQRFPRELIEHLGARGVFSEKWCGGML
PDVGKLVELAPALGRLSSAGIGVGVSLHDSAIAVLRRFGKSDYLRDICERAIAGQAVLCI
GASEESGGSDLQIVRTEMSSRDGGFDIRGVKKFVSLSPIADHIMVVARSIDHDSASKHGN
VALIAVPTSQASVQRPYAKVGAGPLDTAAVHIDTWVPADALVARAGTGLAAISWGLAHER
MSIAGQIAASCQRAIGITLARMMTRRQFGRTLFEHQALRLRMADLQARVDLLQHGLNGIA
AQGRLDLRAAAGVKVTAARLGEEVMSECMHIFGGAGYLVEETPLGRWWRDMKLARVGGGT
DEVLWELVAAGMAADHGGYRSWGASSA
>MAP1557c (SEQ ID NO: 37) catalyzes the formation of D-ribulose
5-phosphate 3117306: 3118775 MW: 52787.16
MSSSVTPSRPTTGTAQIGVTGLAVMGSNIARNFARHGYTVALHNRSIAKTDALLKEHGDE
GKFVRCETIAEFLDALEKPRRVLIMVKAGDPTDAVINELADAMEPGDIIIDGGNALYTDT
IRREQAMRERGLHFVGAGISGGEEGALNGPSIMPGGPAESYRSLGPLLEEISAHVDGVPC
CTHIGPDGAGHFVKMVHNGIEYSDMQLIGEAYQLLRDALGKTAEQIADVFDEWNSGDLDS
FLVEITAQVLRQTDAKTGKPLVDLILDEAEQKGTGRWTVKSALDLGVPVTGIAEAVFARA
LSGSVAQRRATTGLASGRFGEKPSDAAQFTEDIRQALYASKTIAYAQGFNQIQAGSAEYG
WDITPGDLATIWRGGCIIRAKFLNRIKDAFDENPDLPTLIVAPYFRSAIEAAIDGWRRVV
VTATRLGIPIPGFSSALSYYDALRTERLPAALTQGLRDFFGAHTYGRIDEDPDKRFHTLW
SADRREVPA
>MAP1561c (SEQ ID NO: 38) ndh-3113489: 3114874 MW: 49592.51
MSPHSGSTAGPERRHQVVIIGSGFGGLNAAKKLKHANVDIKLIARTTHHLFQPLLYQVAT
GIVSEGDIAPPTRVVLRRQRNVQVLLGDVTHIDLAGKFVVSDLLGHTYETPYDTLIVAAG
AGQSYFGNDHFAEFAPGMKSIDDALEVRGRILSAFEQAERSRDPERRAKLLTFTVIGAGP
TGVEMAGQIAELATYTLKGSFRHIDPTKARVILLDAAPAVLPPFGDKLGKRAADRLEKMG
VEIQLGAMVTDVDRNGITVKDSDGTVRRIESACKVWSAGVSASPLGRDLAEQSTVELDRA
GRVKVLPDLSIPGHPNVFVIGDLAAVEGVPGVAQGAIQGAKYVANTIKAELGGADPAERE
PFQYFDKGSMATVSRFSAVAKIGPLEFSGLFAWFAWLVLHLVYLVGFKTKVSTLLSWTVT
FLSTRRGQLTITEQQAFARTRLEQLAVLAAETKRPAARRAS
>MAP1569 (SEQ ID NO: 39) modD-1723216: 1724322 MW: 36116.12
MDQVEATSTRRKGLWTTLAITTVSGASAVAIALPATSHADPEVPTPVPPSTATAPPAAPA
PNGQPAPNAQPAPGAPAPNGQPAPAAPAPNDPNAAPPPVGAPPNGAPPPPVDPNAPPPPP
ADPNAGRIPNAVGGFSYVLPAGWVESDASHLDYGSALLSKVTGPPPMPDQPPPVANDTRI
VMGRLDQKLYASAEANNAKAAVRLGSDMGEFFMPYPGTRINQDSTPLNGANGSTGSASYY
EVKFSDASKPNGQIWTGVIGSANGGNAQRWFVVWLGTSNDPVDKVAAKALAESIQAWTPP
AAPPAAPGGPGAPAPGAPGTPAAPGAPAAPAPAAPGAPAAPGAPAPGQAPAVEVSPTPTP
TPQQTLSA
>MAP1591 (SEQ ID NO: 40)-1748688: 3749389 MW: 25432.129
MEKVIAVLMRADSEEDWCARQRGVVADALLELGLPGLAVNVRDDAVRRSLMTLTTLDPPV
AAVVSMWTQQSYGEQVAAALRLLAAECEQLAAYLVTESVPLPAPQTEPASRTPGLANTAL
LRRPAGMDQETWLTRWQRDHTPVAIETQSTFGYTQNWVVRTLTPGAPEIAGIVEELFPAE
AITDLQAFFGAADEQDLQHRLGRMVASTTAFGANENIDTVPTSRYVVKTPFAQ
>MAP1651c (SEQ ID NO: 41)-3024459: 3025202 MW: 26097.928
MTQIAFLAYPGFTALDMIGPYEVLRNLPGAEVRFVWHETGPITADSGVLVIGATHSLAET
PSPDVILVPGGPGTAVHARDDALLDWLPAAHRTATWTTSVCTGSLILAAAGLLDGRRATS
HWLTIPALKAFGVTAVPDERIVHEDGIVTSAGVSAGLDIALWLAAQTGGDGRAKAIQLAL
EYDPQPPFDSGHLSKASASTKAAATALLSRDSLSPTYLKATALLAWDQALDRVRSRRRRR
QPDLSPA
>MAP1761c (SEQ ID NO: 42)-2905253: 2906506 MW: 43642.17
MVRRIAGATCRSRESAWPAAVLVATTMLSVTACGHSGDNANHAAQSKPGGGNAVKITLTN
SAGKDGCALDTTNVPAGPVTFTVANTNAPGISEVELLRDORIVGEKENLAPGLDPVSFTL
TLDGGSYQLYCPGASTEYQTLTVTGKAPATPTGTIATVLSQGTKDYAAYIVNQIGQLNDG
AKALDAAVQAGNLDAAKAAYAKARLYWERSESTVEGFVLPGFAVGDNAGNLDYLIDMRES
TPVDGKVGWKGFHAIERDLWQAGAITPGTKALSTELVGNVGKLHGIVATLQYKPEDLANG
ASDLIEEIQNTKITGEEEAFSHIDLVDFSGNVEGAQQAYASLRPGLEKIDNNLVHQIDQQ
FQNVLATLDGYRDPGALGGYRTYTPALKASDAPKLTAVIQPLHQSLSTVAQKVVSAG
>MAP1782c (SEQ ID NO: 43)-2884337: 2885575 MW: 45907.97
LETWVMSISFETSESRADAELPVLPMPRAAHCPLAPPPEFVDWRQQPGLRRALFQGNPVW
VVSRYHDIRAALVDPRLSAKTIPDSIMPTDADNKVPVMFARTDDPEHHRLRRMLTGNFTF
RRCESMRPQIQDTVDHYLDRMLDGGAPADLVREFALPVPSLVIALLLGVPPEDLELFQFN
TSKGLDQKSSDEEKGKAFGAMYAYIEELVQRKAREPGDDLISRLITEYVATGQLDHATTA
MNSVIMMQAGHETTANMISLGTVALLGNPEIYARLGQTDDSAVVANIVEELMRYLSIVHS
QVDRVATEDLTIAGQLIRAGEFVVMNLPAGNWDTEFVDNPESFDADRNTRGHLGFGYGVH
QCIGANLARVEMQVAFATLARRLPGLRLAVPPEQLKFKDANIYGMKELPVSW
>MAP1922c (SEQ ID NO: 44) - 2706005: 2707156 MW: 41258.727
VLVVSTDQAHSLGDVLGVPVPPSQAELVRVLADLETGRAEAGGGFLDALALDTLALLEAR
WRDVVATLDRRFPDSELSTIAPEELSALPGVQEVLGLHAVGELARSGRWDRVVVDCASTA
DALRMLTLPATFGLYVERAWPRHRRLSLTAEDARSAAVVELLERVSASVEALSALLTDGD
LVGAHLVLTPERVVAAEAARTLGSLALMGVRVEELIVNQVLLQDDSYEYRNLPEHPAFYW
YTERIAEQQSVLEELDAAIGEVALVLTPHLSGEPIGPKALGALLDAARRRGGAAPPGPLR
PTVDLESGTGLGSIYRMRLALPQLDPSALTLGRVDDDLIISAGGLRRRVRLASVLRRCTV
LDAHLRGSELTVRFRPDPEVWPK
>MAP1960 (SEQ ID NO: 45)-2163747: 2164499 MW: 26962.055
MAKSRSAADNKAARAQAQAARKAAARERRAQLWQAFNIQRQEDKRLLPYMIGAFLLVVGV
SVGVGVWAGGLTMITLIPFGVVLGALVAFIVFGRRAQKSVYRKAEGQTGAAAWALDNLRG
KWRVTPGVAATGHFDAVHRVTGRPGVTLVGEGSPTRVRPLLAQEKKRTARLIGDVPTYDT
IVGNGEDEVPLAKLERHLTRLPANITVKQMDTLESRLAALGSRAGAAVMPKGPLPNAGKM
RGVQRTVRRK
>MAP1968c (SEQ ID NO: 46)-2653728: 2655290 MW: 55454.46
VGMGLSRRGKSARTLLIWMSIAAVALLLAGCVRVVVGRAVMSGPKLGQAVEWTPCRAANP
KVKLPAGALCGKLAVPVDYDHLDGDVATLAMIRFPATGDKIGSLVINPGGPGESGIEAAL
GVVQSLPKRVRERFDLVGFDPRGVGASRPAVWCNSDADNDRLRTEPNVDYSPAGVAHIED
ETKQFVGRCVDKMGKKFLANVGTVNVARDLDAIRAALGDDKLTYLGYSYGTRIGSAYAEA
YPHNVRAMILDGAVDPNADQIEADLRQAKGFQDAFNNFAAECAKQPNCPLGTDPAKAVDV
YHSLVDPMVDPDNPMVGRPIPTNDPRGLSYSDAIVGTIMALYSPNLWHHLTDGLSELVDH
HGDTLLALADMYMRRDAHGHYTNATDARVAINCVDQPPITDRAKVIDEDRRSREIAPFMS
YGQFTGNAPLGTCAFWPVPPTSKPHTISAPGLAPTVVVSTTHDPATPYKAGVDLANELRS
SLLTYDGTQHTWFQGDGCIDNYVTAYLVGGTIPPSGAKC
>MAP1986 (SEQ ID NO: 47)-2191684: 2192511 MW: 29947.895
MPRWLRGLSFLLRPGWVVLALVVVAFAYLCFTVLAPWQLGKHSRTSQQNHQIEHSLTTPP
VPLKTLLPQQNSAAPAEQWRQVSATGHYLADVQVLARLRVIDSKPAFEVLAPFVVDGGPT
VLVDRGYVRPLEGSRVPPIPRPPADTVTITARLRNSEPAAGKDPFVGDGVRQVYSIDTEQ
IAVLTKVPLAGSYLQLVDGQPGGLGVVGVPQLDAGPFLSYGIQWIAFGILAPIGVGYFAY
SELRARRAERQPAAPAPEAPQSVQDKLADRYGRRR
>MAP2093c (SEQ ID NO: 48) rocE−2514169: 2515620 MW: 50562.71
LPATPIGLRAQLLRRRPVVGAHVAPGTADHLRRGIGTFQLTMFGVGSTIGTGIFFVMSQA
VPEAGPAVIVSFLLAGVAAGLAAVCYAELASAVPVSGSSYSYAYTTLGEVVAMGVAACLL
LEYGVATAAVSVNWSGYLNKLLSNVVGFQLPHALSAAPWDAQPGYVNLPAVMLIGMCALL
LIRGASESAKVNAIMVMIKLGVLVVFGILAFTAFDVHHLDDFAPFGVAGVGTAAGTIFFS
YIGLDAVSTAGDEVTNPQKTMPRALIAALSTVTGVYVFVALAALGTQPWQDFGGQQEAGL
ATILDHVTHGSWASTILAAGAVISIFSVTLVTMYGITRILFAMGRDGLLPPRFARVNPRT
MTPVNNTVIVAVAASTLAAFIPLQNLADMVSIGTLTAFVVVSVGVIVLRVREPDLPRGFR
VPGYPVTPVLSIMACGYILASLHWYTWIAFSGWVLLALIFYFVWGRHHSALNDAAVDPSG
QER
>MAP2100 (SEQ ID NO: 49)-2.322292: 2324052 MW: 62291.344
MITSKLRAQRPSFRTDEANSTHRLPLRTAARTTGVVAYQLGLSVDGHETLSGISFTAKPG
TMTAVIGPSPARNAALLALLAGTRTPSSGRVTVDGHDVHAEPAAMRARIGVVSREERLHR
RLTVEQALRYAAELRLPPETSAEQRDRVVGQVLDELDLTTHRDTRIRKLAPEVRRCTALA
IELVTRPSLLVVDEPTAGLNAAQQRHVMAVLRRQANLGCVVVAAISSRTSLTDVNMCDQV
LVLTAAGKVAYLGTPLQAESAMGSADWSAVLARVGADPDGAHRAFRARPQSAAPTIPPEV
AAPWAPPAALPVPRQVRCVARREIRLLLANRLYFAFLALLPFVLAGLTLLIPGDSGLARP
APSSANAHEAIEILALLNVAAVIIGTALTVPAMVGEHRVYRREQQVGLSAPAYLAAKIAV
YALAAAVWAAVMLAVVIAVKGAPVYGAVVLHDATFELYVAVAVTAMVSAVIGLALSALGK
SLGEVLPLLVPVILAAVLFNGSLVQLVSMWGLQQISWLIPARWGFAASASTVNLRRIDPL
AANAETWTHYSGWWVFDMVMLVLFGVAAAGVTLYRLRSPGKIRSAT
>MAP2117c (SEQ ID NO: 50)-2484475: 2485242 MW: 26255.744
VNATAIAKPMTALGQFFLLSAEALAAAVRGPWAWREILEQIWFVARVSIFPT1MLSIPYT
VLIVFVLNILLVEIGAGDLSGAGAGLASVTQVGPVVTAMVVSGAGSTAMCADLGARTIRE
EIDAMKVIGVNPVQALVVPRIIAATFVAVMLYAVVAVIGLTGSYIFVVFVQHVTPGAFVA
GMTLVTGLPQVVISLIKATLFGLSAGLIACYKGLSVGGGPTGVGNAVNETVVFSFMALFF
INILTTALGVKVTAK
>MAP2123 (SEQ ID NO: 51) cysK-2352623: 2353555 MW: 32346.6
MSIAENVTQLIGNTPLVRLNRVTEGAVADVVAKLEFFNPGNSVKDRIGVAMIDAAEQAGL
IKPDTIILEPTSGNTGIALALVAAARGYRCVLTMPETMSVERRMLLRALGASIVLTPGAD
GMPGAIAKAEELAKSDDRYFVPQQFENPANPAIHRSTTAEEVWRDTDGKVDIFVAGVGTG
GTITGVAQVIKERKPSAQFIAVEPAASPVLSGGQKGPHPIQGLGAGFVPPVLAMDLVDEV
IAVGNEESIALARRLAAEEGLLVGISSGAALVAALQVARRPENAGKLVVVVLPDFGERYL
STPLFADLAD
>MAP2158 (SEQ ID NO: 52)-2390352: 2390933 MW: 21032.867
MDQDDLPRTARVSIVAPSPEGELAEVALLFTNIVRRDTAAFREELQNLVNSLAETSETKP
VITESQTPYPGGGLAQYGIAFAVGLPTALAYNVIYDALKKLSHRFSWTAGSPPQERFLME
NANPLALGAIEQGFGVARDDLRPVVVDVQGLRAHVVYHAKDGSMFTVEMENTGQFAITSV
RKNWPNAGWGDES
>MAP2187c (SEQ ID NO: 53) - 2400196: 2401278 MW: 37727.773
LRVELLVKIEYGSTVTWYLGVVVTIVAEQRTYVAGRWVTGDEVVSVENPADSSHVADITV
TPLPEVQRAIAEARRSFDDGVWADMPPVERAQILHAFIDHIESERATLVPTLVAEAGQSA
RFAEMTQLGAGAAIARQTIDLYLSMSHEEASPVPVDDLVRGRVALSVRRHEPVGVVTAIT
PYNAALIMGFQKLIPALMAGNSVILRPSPLTPISSLIFGAAADAAGLPPGVLSVVVESGI
AGAELLTSDPSVDMVSFTGSTLAGRKILAQAAPTVKRVSLELGGKSAQIYLPDAVHRAVG
GAEVAVASTAGQACVAATRLLVPQDKKAEVLDAVSAMYQQIKVGPPSDETAMMGPVISAA
>MAP2195 (SEQ ID NO: 54)-2439276: 2440622 MW: 49261.3
MGLLGCRRIWKGPTRRLVLRRRWRSRSSGSVKHPCGPGRRRPGVADSEFVVASPAGDTVD
QIDTVPIDSGASVPPSGNPVSLIAAACCEHRTNVDPEVQTQVAIEEWMGASPNYTRRLRH
ALGVTGDTVEDIFKVLQFDVGAPPQFLDFRYSLTDPNHGEFRNDYCGALIDVEPMGDVWV
RAMCHTIQDFTFDATAIATNPKARFRPIHRPPRKPADRTPHCHWSVTIEDSREDLPIPAE
AVEVSRCELTALQFDPIDLSDDGLGDYTGPLFSDIRFDQWSRSALVRLAEEVAIQHHLLA
LAFERSVRRHGGEAKALGLLRRQFTGTAYVGSARIKAASGLASAQMTLLRSSICIPPCAR
SPTPEHPWSASGQERATRCGCASQATPPPSATAVGWRRCRRTMSVPSRSWPPVSIRTGRI
CTRPTRPATWSSTSGGRTPKRSAGRKSK
>MAP2271c (SEQ ID NO: 55) valine tRNA ligase; ValRS;
converts valine ATP an-2290752: 2293400 MW: 98912.45
VTASRSPATDLPKSWDPPAAEYAIYRQWVDAGYFTANPASDKPGYSIVLPPPNVTGSLHM
GHALEHTMMDALTRRKRMQGYEVLWQPGMDHAGIATQSVVEKQLAVDGKTKEDFGRELFI
EKVWDWKRESGGAIGGQMRRLGDGVDWSRDRFTMDEGLSRAVRTIFKRLYDAGLIYRAER
LVNWSPVLQTALSDIEVNYEEVEGELVSFRYGSLDDSGPHIVVATTRVETMLGDTAIAVH
PDDERYRHLVGSSLPHPFVDRQLLIVADEHVDPEFGTGAVKVTPAHDPNDFEIGLRHQLP
MISIMDTRGRIADTGTQFDGMDRFAARVAVREALAAQGRIVEEKRPYLHSVGHSERSGEP
IEPRLSLQWWVRVESLAKAAGDAVRNGDTVIHPTSMEPRWFAWVDDMHDWCVSRQLWWGH
RIPIWYGPNGEQRCVGPDETPPEGWEQDPDVLDTWFSSALWPF3TLGWPEKTPELEKFYP
TSVLVTGYDILFFWVARMMMFGTFVGDDDAITLDGRRGPQVPFTDVFLHGLIRDESGRKM
SKSKGNVIDPLDWVDMFGADALRFTLARGASPGGDLAIGEDHVRASRNFCTKLFNATRYA
LLNGAQIAELPPLDELTDADRWILGRLEEVRAEVDSAFDNYEFSRACESLYHFAKDEFCD
WYVELAKTQLAEGITHTTAVLATTLDTLLRLLHPVIPFITEALWQALTGNESLVIADWPR
SSGIDLDQVATQRITDMQKLVTEVRRFRSDQGLADRQKVPARLAGVTESDLDTQVSAVTS
LAWLTDAGPDFRPSASVEVRLRGGTVWELDTSGSIDVAAERRRLEKDLAAAHKELASTT
AKLANEDFLAKAPPHWDKIRDRQRLAQEESERINARLAVLQ
>MAP2288c (SEQ ID NO: 56) - 2269954: 2270430 MW: 16234.525
VAPVARGEVATREPAELPNGWVITTSGRISGVTEPGELSVHYPFPIKDLVAIDDALKFGS
RASKTRFAIYLGDLGTDTAARAREILADVPTPDNAVLLAVSPDQKVIEVVYGSAVRGRGA
ESAAPLGVAAASSAFQRGDLVDGLVSAIRVLSAGISPA
>MAP2424c (SEQ ID NO: 57) converts L-glutamate to D-glutamate,
a component o-2107951: 2108778 MW: 29114.562
MSSALAFVGIFDSGVGGLTVARAIIDQLPDEHIIYVGDTGHGPYGPLSIPEVRAHALAIG
DDLVGRGVKALVIACNTASAACLRDARERYEVPVVEVILPAVRRAVATTRNGRIGVIGTQ
ATINSHAYQDAFAAARDTEITAVAGPRFVDFVERGVTSGRQVLGLAEGYLEPLQRAQVDT
LVLGCTHYPLLSGLIQLAMGDNVTLVSSAEETAKEVLRVLAERDLLHPHPDDPRAAGPSR
VFEATGDPEAFTRLAARFLGPAVSGVRPVHKVRID
>MAP2447c (SEQ ID NO: 58) adds enolpvruvyl to UDP-N-
acetylglucosamine as a c-2075358: 2076611 MW: 43976.785
VAERFVVTGGNRLSGEVAVGGAKNSVLKLMAATLLAEGTSTITNCPDILDVPLMAEVLRG
LGATVELDGDVARITSPDEPKYDADFAAVRQFRASVCVLGPLVGRCKRARVALPGGDAIG
SRPLDMHQAGLRQLGATCNIEHGCVVAQADTLRGAEIQLEFPSVGATENILMAAVVAEGV
TTIHNAAREPDVVDLCTMLNQMGAQVEGAGSPTMTITGVPRLYPTEHRVIGDRIVAATWG
IAAAMTRGDISVTGVDPAHLQVVLHKLHDAGATVTQTDDSFRVTQYERPKAVNVATLPFP
GFPTDLQPMAIALASIADGTSMITENVFEARFRFVEEMIRLGADARTDGHHAVVRGLPQL
SSAPVWCSDIRAGAGLVLAGLVADGDTEVHDVFHIDRGYPLFVENLAILGAEIERVE
>MAP2448 (SEQ ID NO: 59)-2754503: 2755102 MW: 21289.205
LVMAVHLTRIYTRTGDDGTTGLSDFSRVSKNDPRLVAYADCDEANAAIGVAVAVGRPGPE
LAGVLRQIQNDLFDAGADLSTPVVEDPEYPPLRVTQPYIDRLEKWCDTYNESLPKLNSFV
LPGGSPLSALLHVARTVVRRAERSAWAAVDAAPEGVSALPAKYLNRLSDLLFILSRYANP
DGDVLWKPGGQQGGEPAPG
>MAP2497c (SEQ ID NO: 60) IprC-2024924: 2025493 MW: 20164.4
MMAMMRPGPRRSTARAAATVLFLALLVLTGCSRSIAGNAVKAGGNVPRNNNSQQQYPNLL
KECEVLTSDILAKTVGADPLDIQSTFVGAICRWQAANPAGLIDITRFWFEQGSLSNERKV
AEFLKYKIETRNIAGIDSIVMRPDDPNGACGVASDAAGVVGWWVNPQAPGIDACGQAIKL
MELTLATNS
>MAP2609 (SEQ ID NO: 61)-2941423: 2941755 MW: 11397.098
MRLSLSKLGVAVGSAAVALTAAAGVASADPMDAIINTTCNYGQVIAALNASDPAAAQQLN
SSPMAQSYIQRFLASPPAKRQQMAQQIQGMPAAQQYINDINQVAVTCNNF
>MAP2694 (SEQ ID NO: 62)-3029482: 3030537 MW: 34768.29
VTAVDDSKDGFSMTAPPGGIYGPGSYGSNPYGQEPNWGGQPPGGQPPGGQPQGGPYPQPG
QYPAGGPYPYPPPGGGYPYPGGPYPGGPYPGAPYPGPGQPFGPGGPYSPGPPPGGPGSKL
PWLIVAGLVVLAVIALVATLVVMKGGHGSKPSGATPSSTSTSVSQPKNSAQNATDCTPNV
SGGDMPRSDSIAAGKLSFPANAAPSGWTVFSDDQGPNLIGALGVAQDVPGANQWMMTAEV
GVTNFVPSMDLTAQATKLMQCLANGPGYANAMPTLGPIKTSPITVDGTKAVRADADVTIA
DPTRNVKGDSVT11AVDTKPVSVFIGSTPIGDSASAGLIGKIIAALKVAKS
>MAP2837c (SEQ ID NO: 63)-1663191: 1665551 MW: 81999.97
LEQSNASPATRRIVSGSFPRAIAARSPETQYGRRRKGSHARRLSGLVKVHRGPMRKLVGS
ALVSLTTTALAAVLLAPAATASPIGDAEAAIMAAWEKAGGDTSPLGARKGDVYPVGDGFA
LDFDGGKMFFTPATGAKFAYGPILDKYESLGGPAGSDLGFPAINEVPGLAGPDSRVVTFS
ASDKPVIFWTPEHGAYVVRGAINSAWDKLGSSGGVLGVPVGDETYNGEVSTQKFSGGQVS
WNRQTKOFSTEPPGLADQLKGLQVAIDPTAAINTAWRAAGGPGGPLGAKQGGPTPVGGDG
IVQNFAGGKVFFTPATGANALESDILAKYESLGGPAGSDLGFPTTNETDGGIGPSSRIAT
FSAPDKPVIFWTADHGAFVVRGAMRAAWDKLRAPAGKLGAPVGDQAVDGDVTSQQFTGGK
ISWNRAKNAFSTDPSNLAPLLSGLQISGQNQPSSSAMPAHPKKFSWHWWWLMAAVPVAVX
LVLLIWVLFVWRRRRPGPEATGYGVDKGYDAAEGQWGHDDADVATEHFGAPPSGEPPAGS
GAAARVSWQRQAPADGGYGFEEEDPDAVDTDSIPVVSDEMLAEADYPAAEADYTDYTDAV
PEVAEPETADDAAYADADYAEVDYPDVGYREDEYPDLAVPHTPPDADAVTGGIPAAEADD
EYAELAAPQAQPEERPEPQPGPEEVAEAAGGAVAAGVAGTRPRSGRKAAADEEDASENGL
AGPDGRPTIHLPLEDPYQAPEGYPIKASARYGLYYTPGSDLYRDTLPELWLSSEEVAQAN
GFTKAD
>M AP2875 (SEQ ID NO: 64)-3205118: 3205960 MW: 29724.037
VIAVTIEDPAIMPEAFFTVDGDSYVPGTMTRGPWGAAMGGQIVGGLLGWGIEQSGVDPDL
QPARFTVDLLRPALLAPVQIRTSVQREGRRIKLVDAGLVQNGVVVARASALFLRRGDHPD
GQVWSPPVQMPPLPTSSEGFPADMPFLIWGYGATRAGSPGIAAGEWEQAHSQKFAWARLF
RPMVHGHPLTPFTRLAFVGDITSSLTHWGTGGLRYINADYTVSASRLPDGEFLGLAAQSH
YGTAGVAAGAATLFDRHGPLGTSWALALAQPADAFQPAYT
>MAP2891c (SEQ ID NO. 65) gpsI-1606721: 1608994 MW. 80543.2
MSVAEIEEGVFEATATIDNGSFGTRTIRFETGRLAQQAAGAVVAYLDDENMLLSATTASK
SPKEHFDFFPLTVDVEERMYAAGRIPGSFFRREGRPSTDATLTCRLIDRPLRPSFVDGLR
NEIQVVVTILSLDPNDLYDVLAINAASASTQLGGLPFSGPIGGVRVALIDGTWVAFPTVE
QLERAVFDMVVAGRKVDGADGPDVAIMMVEAEATSNVIELIDGGAQAPTETVVAQGLEAA
KPFIEVLCTAQQELADKAARPTSDYPTFPDYGDDVYYSVASVATDELSKALTIGGKAERD
ARTDELKAEVLARLAETYEGREKEVSAAFRSLTKKLVRQRILTDHFRIDGRGITDIRALS
AEVAVVPRAHGSALFQRGETQILGVTTLDMVKMAQQIDSLGPETTKRYMHHYNFPPFSTG
ETGRVGSPKRREIGHGALAERALVPVLPSLEDFPYAIRQVSEALGSNGSTSMGSVCASTL
ALLNAGVPLKAPVAGIAMGLVSDDIEVEAGDGTKSLERRFVTLTDTLGAEDAFGDMDFKV
AGTKDFVTALQLDTKLDGIPSQVLAGALSQAKDARLTILEVMAEAIDEPDEMSPYAPRVT
TIRVPVDKIGEVIGPKGKIINAITEETGAQISIEDOGTVFVGATDGPSAQAAIDRINAIA
NPQLPTVGERFLGTVVKTTDFGAFVSLLPGRDGLVHISKLGKGKRIAKVEDVVNVGDKLR
VETADIDKRGKISLVLVEEDNSAPADTPAAAPADATS
>MAP2923 (SEQ ID NO: 66) catalyzes the reduction of mycothione
or glutathio-3256478: 3257857 MW: 49811.098
METYDLAIIGTGSGNSLLDARFAGKRTAICEHGTFGGTCLNVGCIPTKMFVYAADVATTI
REAARYGVDTHLDGVRWPDIVSRVFGRIDPIALSGEEYRRSSVNIDLYRSHTRFGPVQFD
GRYLLRTDAGEQFTAEQVVIAAGSRPVIPPAILESGVTYHTSDTIMRIPALPEHLVIVGS
GFVAAEFAHIFSALGVHVTVVIRSGRMLRQYDDMICERFTRLAAAKWELRTQRNVVGGSN
RGSGVTLRLDDGSTLDADVLLVATGRISNADLLDAGQAGVDVENGRVVVDEYQRTSARGV
FALGDVSSPYQLKHVANHEARVVRHNLLCDWDDTESMAVTDHRYVPSAVFTDPQIATVGL
TENQAIARGFDISVAIQNYGDVAYGWAMEDTTGVVKLIAERTSGRLLGAHIMGPQASSII
QPLIQAMSFGLTAAQMARGQYWIHPALPEWENALLGLY
>MAP2931 c (SEQ ID NO: 67) glnA4-1563611: 1564996 MW: 49747.95
LTGSDTAMLSLAALDRLVAAGAPETRVDTVIVAFPDMQGRLVGKRMDARLFVDEAAATGV
ECCGYLLAVDVDMNTVGGYAISGWDTGYGDLVMRPDLSTLRRIPWLPGTALVIADVVGAD
GSPVAVSPRAVLRRQLDRLAGRGLFADAATELEFMVFDEPYRQAWASGYRGLTPASDYNI
DYAISASSRMEPLLRDIRRGMAGAGLRFESVKGECNRGQQEIGFRYDEALRTCDNHVIYK
NGAKEIADQHGKSLTFMAKYDEREGNSCRVHLSLRDAQGGAAFADPSRPHGMSTMFCSFL
AGLLATMADFTLFYAPNINSYKRFADESFAPTALAWGLDNRTCALRVVGHGAHTRVECRV
PGGDVNPYLAVAAIVAGGLYGIEQGLALPEPCAGNAYRARGVGRLPGTLAEAAALFEHSA
LARQVFGDDVVAHYLNNARVELAAFHAAATDWERMRGFERL
>MAP2942c (SEQ ID NO: 68) mpt53-1551608: 1552126 MW: 18261.871
VRLQGMSRLSFVCRLLAATAFAVALLLGLGDVPRAAATDDRLQFTATTLSGAPFNGASLQ
GKPAVLWFWTPWCPYCNAEAPGVSRVAAANPGVTFVGVAAHSEVGAMANFVSKYMLNFTT
LNDADGAIWARYGVPWQPAYVFYRADGSSTFVNNPTSAMPQDELAARVAALR
>MAPS039c (SEQ ID NO: 69)-1448221: 1448679 MW: 15676.337
LGTRPALARVSSGSVANVTAGRRSSSPLFRRARAQEPRWKRSPSMSSQRTVRLSSSVRTV
TPRSSATVRNRTISAESSTGSSSNGADGGQSITGISRPKVKNPTARGATGDTLITTGAAA
>MA.P3112c (SEQ ID NO: 70)-1369027: 1370484 MW: 53668.797
MNAEPRTGPAKTLASALARDIEAEIVRRGWAVGESLGSEPALQQRFGVSRSVLREAVRLV
EHHQVARMRRGPNGGLYICEPDAGPATRAVVIYLEYLGTTLADLLNARLVLEPLAASLAA
ERIDEAGIARLRAVLHAEQQWRPGLPMPRDEFHIALAEQSKNPVLQLFIKVLMRLTTRYA
LQSRTDSETEALEAVDHLHTHHSRIVAAVTAGDPARAKTLSERHVEAVTAWLQRHHAGDR
NRGRTPRRPLNSEVPQGKLAEMLAATIGDDIAADGWRVGSVFGTETALLQRYRVSRAVFR
EAVRLLEYHSIAHMRRGPGGGLVIAEPAAQASIDTIALYLQYRDPSREDLRCVRDAIEID
NVAKVVKRLAEPQVAAFVASRRSGLPDDSRQTPDDVRRAIAEEFDFHVGLAQLAGNAPLD
LFLRIIVELFRRHWSSTGQALPTWSDVRAVHHAHLRIADAVAAGDLSVASYRLRRHLDAA
ASWWL
>MAP3305c (SEQ ID NO: 71)-1152824: 1153678 MW: 30720.326
VTVEPPPDHVLSAFGLAGVKPVYLGASWEGGWRCGEVVLSLVADNARAAWSARVRETLFV
DGVRLARPVRSTDGRYVVSGWRADTFVAGTPEPRHDEVVSAAVRLHEATGKLERPRFLTQ
GPTAPWGDVDIFIAADRAAWEERPLASVPPGARVAPATADAQRSVELLNQLATLRKPTKS
PNQLVHGDLYGTVLFVGSAAPGITDITPYWRPASWAAGVVVIDALSWGEADDGLIERWNA
LPEWPQMLLRALMFRLAVHALHPRSTAEAFPGLARTAALVRLVL
>MAP3399 (SEQ ID NO: 72) accD5-3775092: 3776732 MW: 59081.77
MTSVTDHTAEPAAEHSIDIHTTAGKLAELHKRREESLHPVGEEAVEKVHAKGKLTARERI
LALLDEDSFVELDALARHRSKNFGLENNRPLGDGVITGYGTIDGRDVCIFSQDATVFGGS
LGEVYGEKIVKVQELAIKTGRPLIGINDGAGARIQEGVVSLGLYSRIFRNNILASGVIPQ
ISLIMGAAAGGHVYSPALTDFVVMVDQTSQMFITGPDVIKTVTGEDVTMEELGGAHTHMA
KSGTLHYVASGEQDAFDWVRDLLSYLPPNNATDPPRYAEPHPAGAIEDNLTDEDLELDTL
IPDSPNQPYDMHEVITRILDDDEFLEIQGGYAQNIVVGFGRIDGRPVGIVANQPTQFAGC
LDINASEKAARFVRTCDCFNIP11MLVDVPGFLPGTGQEYNGIIRRGAKLLYAYGEATVP
KITVITRKAYGGAYCVMGSKDMGCDVNIAWPSAQIAVMGASGAVGFVYRKQLAEAAKKGE
DVDALRLQLQQEYEDTLVNPYVAAERGYVDAVIPPSHTRGYIATALRLLERKIAHLPPKK
HGNIPL
>MAP3401 (SEQ ID NO: 73) Maf; overexpression in Bacillus
subtilis inhibits-3776986: 3777618 MW: 21244.516
LTRLVLASASAGRLKVLRQAGVDPLVVVSGVDEDAVIAALGPDASPSAVVCALATAKADR
VAGALQAGVAADCVVVGCDSMLFIDGGLCGKPGSADAALRQWRRIGGRSGGLYTGHCLLR
LRDGDITHREVESACTTVHFASPVEADLRAYVAGGSPLAVAGGFTLDGLGGWFVDGIDGD
PSNVIGVSLPLLRTLLTRVGLSVSALWAAD
>MAP3429 (SEQ ID NO: 74) catalyzes the formation of a purine
and ribose pho-3808372: 3809187 MW: 27823.328
VAETPSNPGELARQAAAVIGERTGVAEHDVAIVLGSGWSPAVAALGTPTAVLPQAELPGF
RPPTAVGHTGELVSMRIGEHRVLVLVGRIHAYEGHDLCHVVHPVRAACAAGVRAVVLTNA
AGGLRPDLAVGEPVLISDHLNLTGRSPLVGPQFVDLTDAYSPRLRELARQADPTLAEGVY
AGLPGPHYETPAEIRMLRTLGADLVGMSTVHETIAARAAGAEVLGVSLVTNLAAGISGEP
LSHTEVLAAGAASATPMGALLALILCQLPRF
>MAP3490 (SEQ ID NO: 75)-3880292: 3881887 MW: 53711.008
VTTVPAMTAPIWMASPPEVHSALLSSGPGPASMFAAAAAWSALGAEYASAAEELSGLLAS
AQAGAWQGPSAASYVAAHGPYLAWLTRASAHSAAAAAQHETAGTAYTAALAAMPTLPELA
ANHAVHGALVATNFFGINTIPIAVNEADYAFMWVQAAGTMATYQAVSTAAVAAVPQPDPA
PSILKSTAAHDHDDHEHGDDHDHDHGFDSPLNQFVAQILRLFGIDWDPVEGTLNGLPYEA
YTSPADPLWWVVRALELFSDFQQFGALLQENPAAAFQFITELVLLDWPTHLAQLASWLPT
QPQLLLVPALVAAAPFGALAGFAGVAGQPPLPAPVAEPATPSAAAPTGLPATAGATPIAA
SAAASGPAPAPTPAPTAATVSSPAPPAPPAPGAAPFAPPYAVPPPGAGFGSKARASVDTR
AKSKSPQPDSNAVGAGAAVREAAHARRRQRSRRRGDEFMDMNVGVDPDWDEPATTASPRG
AGNLGFAGTAPRETVAAAGLTQLAGDEFGGGAGMPLLPGSWAPPDPRDSGV
>MAP3527 (SEQ ID NO: 76) pepA-3929882: 3930967 MW: 35709.477
MSKSHHHRSVWWSWLVGVLTWGLGLGLGSGVGLAPASAAPSGLALDRFADRPLAPIDPS
AMVGQVGPQVVNIDTKFGYNNAVGAGTGIVIDPNGVVLTNNHVISGATEISAFDVGNGQT
YAVDVVGYDRTQDIAVLQLRGAAGLPTATIGGEATVGEPIVALGNVGGQGGTPNAVAGKV
VALNQSVSATDTLTGAQENLGGLIQADAPTKPGDSGGPMVNSAGQVIGVDTAATDSYKMS
GGQGFAIPIGRAMAVANQIRSGAGSNTVHIGPTAFLGLGVTDNNGNGARVQRVVNTGPAA
AAGIAPGDVITGVDTVPINGATSMTEVLVPHHPGDTIAVHFRSVDGGERTANITLAEGPPA
>MAP353Ic (SEQ ID NO: 77) fbpC2-893667: 894725 MW: 37768.805
MSFIEKVRKLRGAAATMPRRLAIAAVGASLLSGVAVAAGGSPVAGAFSKPGLPVEYLEVP
SPSMGRNIKVQFQGGGPHAVYLLDGLRAQDDYNGWDINTPAFEEFYQSGLSVIMPVGGQS
SFYSNWYQPSSGNGQNYTYKWETFLTQEMPLWMQSNKQVSPAGNAAVGLSMSGGSALILA
AYYPQQFPYAASLSGFLMPSEGWWPTLIGLAMNDSGGYNANSMWGPSTDPAWKRNDPMVQ
IPRLVANNTRIWVYCGNGTPSDLGGDNVPAKFLEGLTLRTNEQFQNNYAAAGGRNGVFNF
PANGTHSWPYWNQQLMAMKPDMQQVLLSGNITAAPAQPAQPAQPAQPAQPAT
>MAP3540c (SEQ ID NO: 78)-886065: 886766 MW: 25187.8
MDAVDPDSRHOLAVRMAELVRGMAAPRRLDQVLAEVTAAAVEVIPGADIAGVLLVRKGGE
FETLADTDSLAARLDVLQHDFGEGPCAQAALQETIVRSDDLRREPRWPRYAPAAVQLGVL
SSLSFKLYTADRTAGALNLFSHRPDAWDTEAETIGSVFAAHAAAAILAGSRAEQLYSAVS
TRDRIGQAKGIIMERFGVDDVRAFDLLRRLSQESQVKLVEIAQQIIDTRGQGA
>MAP3573 (SEQ ID NO: 79) pntAB-3971875: 3972192 MW: 11054.443
MYDELLANLAILVLSGFVGFAVISKVPNTLHTPLMSGTNAIHGIVVLGALVVFGSVEHPS
LAMQIILFVAVVFGTLNVIGGFIVTDRMLGMFKSKKPAKADEAAK
>MAP3574 (SEQ ID NO: 80) pntB-3972189: 3973613 MW: 48425.55
MNYLVIGLYIVSFALFIYGLMGLTGPKTAVRGNLIAAVGMAIAVAATLIKIRHTDQWVLI
IAGLVVGVVLGVPPARYTKMTAMPQLVAFFNGVGGGTVALIALSEFIETSGFSAFQHGES
PTVHIVVVSLEAAIIGSISFWGSIIAFGKLQEIISGAPIGFGKAQQPINLLLLAGAVAAA
VVIGLHAHPGSGGVSLWWMIGLLAAAGVLGLMVVLPIGGADMPVVISLLNAMTGLSAAAA
GLALNNTAMIVAGMIVGASGSILTNLMAKAMNRSIPAIVAGGFGGGGVAPGGGDGGDKHV
KSTSAADAAIQMAYANQVIVVPGYGLAVAQAQHAVKDMAALLEEKGVPVKYAIHPVAGRM
PGHMNVLLAEAEVDYDAMKDMDDINDEFARTDVAIVIGANDVTNPAARNEASSPIYGMPI
LNVDKAKSVIVLKRSMNSGFAGIDNPLFYAEGTTMLFGDAKKSVTEVAEELKAL
>MAP3762c (SEQ ID NO: 81)-628824: 630050 MW: 43793.695
MKFALAVYGSRGDVEPHAAIARELLRRGHEVCVAAPPDLRGFVESAGVTAIDYGPDTRDV
LFGKKTNPIKLLSTSKEYFGRIWLEMGETLTSLANGADLLLTAVAQQGLAANVAEYCDIP
LATLHCLPARVNGRLLPNVPSPLSRLAVSAFWCGYWCVTNKAEESQRRRLGLSKASGSST
RRIVGRKSLEIQAYEDFLFPGLAAEWAHWDGQRPFVGALTLGLPTDADAEVLSWIAAGSP
PVYFGFGSLPVKSPADTVAMISAACTRLDERALICAGTNDLTHVPRSGHVKIVAAMNHAA
IFPACRAVVHHGGAGTTAAGMRAGVPTLVLWMRNEQPLWGAAVKQMKVGSSQRFSKTTEE
SLATCLRSILRPHYMTRAREVAKRMTKSSDSAAVAADLLENAARGETT
>MAP3773c (SEQ ID NO: 82)-612529: 612948 MW: 36197.609
VSSPAAPRRRRATVKQRTVLEVLRAQENFRSAQQLYQDIRQNQQLRIGLTSVYRILRALA
ADRIAETQRAEDGEILYRLRTEAGHRHYLLCRQCGRAVAFTPVDIEEHTRRLSRQHHYAD
VTHYVDLYGTCPLCQNTQP
>MAP3852c (SEQ ID NO: 83)-515663: 516250 MW: 19642.443
LSGCSTPSRLSLFRSTLSSFGRPGVRGTRRAMTQTTQPLMRTQVRADIPDSERDPARARR
GGKRVARLRAGAVCWLAIAVCCLAAAGLAATGARTGLGGGSPAPVVPEAGTLQVSGAGTT
KSLPCHAGYLSVSGKDNTVTLTGHCTSVSVSGNGNRIAVDSSDAVSAAGAGNVVVYHWGS
PKVVNAGSGNVVRQG
>MAPS939c (SEQ ID NO: 84)-429862: 430506 MW: 21771.256
VTNPHFAWLPPEVNSALIYSGPGPGPLLAAAAAWDGLAEELASSAQSFSSVTSDLASGSW
QGASSAAMMTVANQYVSWLSAAAAQAEEVSHQASAIATAFEVALAATVQPAVVAANRALV
QALAATNWLGQNTPAIADIEAAYEQMWASDVAAMFGYHADASAAVAKLPPWNEVLQNLGF
SNASTAVTRPAGSGAVARGYTSRIAGFLAPRAPQ
>MAP4074 (SEQ ID NO: 85)-4540713: 4541336 MW: 23135.535
VGWFRFYFEGERWVWSDQVQRMRGYQPGTVTPTTELVLSHKHPADRPQVIDGINDMIRRR
QAFSTRHRIVDTAGIIHHVVVVGDQLFDDSGELVGTHGFYIEVTPAATRNREDSISAKVS
EIAGRRGVIDRTKGMLMLVYGIDEDAAFNMLKSLSQHGNIKLSVLAQRIAEDFTALGKEV
ITARSRFDQRLRTAHLRPPGAGEAGSG
>MAP4143 (SEQ ID NO: 86) EF-Tu; promotes GTP-dependent
binding of aminoacyl-4620946: 4622136 MW: 43739.33
VAKAKFERTKPHVNIGTIGHVDKGKTTLTAAITKVLHDKYPDLNESRAFDQIDNAPEERQ
RGITINISHVEYQTDKRHYAHVDAPGRADYIKNMITGAAQMDGAILVVAATDGPMPQTRE
HVLLARQVGVPYILYALNKADMVDDEELLELVEMEVRELLAAQEFDEDAPVVRVSALKAL
EGDAKWVESVEQLMEAVDESIPDPVRETDKPFLMPVEDVFTITGRGTVVTGRVERGVINV
NEEVEIVGIRPSSTKTTVTGVEMFRKLLDQGQAGDNVGLLLRGIKREDVERGQVVTKPGT
TTPHTEFEGQVYILSKDEGGRHTPFFNNYRPQFYFRTTDVTGVVTLPEGTEMVMPGDNTN
ISVKLIQPVAMDEGLRFAIREGGRTVGAGRVVKIIK
>MAP4144 (SEQ ID NO: 87)-4622313: 4622930 MW: 20633.8
MSFVQATPEFVAAAATDLARIGSTISSANTAALGPTSGVLAPGADEVSASIAALFDAHSQ
VYQALSAQAAAFHSQFVQLMNGGALQYAVTEAANTTPLQSAAGPASVAAQLPAVSGAVGG
SAPYGHPTAPLAAAAGASRYTRDGAGSEHPGGGTQRRGVLGTDSRPDPGQIRRGSRDEFR
SRLNERHRHHPATSYGPRGTTTAKS
>MAP4225c (SEQ ID NO: 88) rmlB-133467: 134462 MW: 37278.766
MRLLVTGGAGFIGANFVHSTVREHPEDSVTVLDALTYAGRRESLAGVEDSIRLVVGDITD
AELVSRLVAESDAVVHFAAESHVDNALAGPEPFLHTNVVGTFTILEAVRRHGVRLHHIST
DEVYGDLELDDPNRFTESTPYNPSSPYSATKAAADMLVRAWVRSYGVRATISNCSNNYGP
YQHVEKFIPRQITNVLTGRRPKLYGTGANVRDWIHVDDHNSAVRRILESGEIGRTYLISS
EGERDNLTVLRTLLQMMGRDPDDFDHVTDRVGHDLRYAIDPSTLYDELCWAPKHTDFEEG
LRETIDWYRANESWWRPLKDASEARYEERGQ
>MAP4231 (SEQ ID NO: 89) located on the platform of the 30S
subunit-4699276: 4699692 MW: 14667.941
MPPAKKAAAAPKKGQKTRRREKKNVPKGAAHIKSTFNNTIVTITDPQGNVIAWASSGHVG
FKGSRKSTPFAAQLAAENAARKAQEHGVRKVDVFVKGPGSGRETAIRSLQAAGLEVGAIS
DVTPQPHNGVRPPKRRRV
>MAP4276 (SEQ ID NO: 90)-4743113: 4743913 MW: 28060.014
VTVPESLDEFARTDLLLDALAQRRPVPRGQVEDPDDPDFQMLTTLLEDWRDNLRWPPASA
LVTPEEAVNALRAGLAERRRGHRGLAVVGSVAATLMLLSGFGAMVVEARPGSTLYGLHAM
FFDQPRVNEKDQVMLAAKADLAKVAESIDKGQWDQARTQLTEVSSLVASIDDPATKQDLM
TQLNLLNAKVDSRNPNATLPAAAPSMAPSVAVPAAPPPAASIAPTPAAPPAPLSPAPAST
PSPSPSVGKHHHHGQPPAVAPVNPNQ
>MAP4339 (SEQ ID NO: 91) trxB2-4819935: 4820945 MW: 35333.918
MTADTVHDVIIIGSGPAGYTAALYTARAQLAPVVFEGTSFGGALMTTTEVENYPGFRDGI
TGPELMDQMREQALRFGADLRMEDVESVSLAGPVKSVTTAEGETVRARAVILAMGAAARY
LGVPGEQDLLGRGVSSCATCDGFFFKDQDIAVIGGGDSAMEEATFLTRFARSVTLVHRRE
EFRASRIMLERARANDKITIVTNKAVEAVEGSETVTGLRLRDTVTGETSTLAVTGVFVAI
GHDPRSELVRDVLDTDPDGYVLVQGRTTATSIPGVFAAGDLVDRTYRQAVTAAGSGCAAA
IDAERWLAEHAESSAAAQGDATEFPGSTDTLIGAPQ
>MAP4342c (SEQ ID NO: 92) - 6367: 7119 MW: 27182.35
VSARITPLRLEAFEQLPKHARRCVFWEVDPAVLGNHDHLADAEFEKEAWLSMVMLEWGCC
GQVATAIPDERSQAEPPCLGYVFYAPPRAVPRAQRFPTGPVSADAVLLTSMGIEPGPAAD
DLPHALLARVIDELVRRGVPALEAFGRTPAASELQDPRLVGPDLRPVLEAVGDCSVDHCV
MDAEFLKDAGFVVVAPHTYFPRLRLELDKGLGWKAEVEAALERLLESARLEQPVGAASTP
ANALKTAPPD
>MAP1201 c+2942c fusion nucleic acid (SEQ ID NO: 93):
TC TAGACGCTCT GATGAGTTGG GCGAGTTCGT TCTGGACCAC GGGGCAGTAG TAATTGCCGC GGTCACCTCG
TGCACGAACA CCTCCAACCC TGAGGTAATG CTTGGGGCTG CGCTTCTGGC GCGTAACGCT GTAGAGAAGG GATTGGCCTC
GAAACCATGG GTTAAGACAA CAATGGCTCC GGGATCGCAA GTTGTCCATG ACTATTATGA CAAGGCGGGG CTGTGGCCTT
ATTTAGAAAA GCTCGGTTTT TACTTAGTGG GCTACGGCTG TACAACGTGT ATTGGAAATT CTGGTCCGTT ACCGGAAGAG
ATCAGTAAAG CAATTAACGA TAATGATTTA TCGGTTACCG CTGTACTGAG TGGCAATCGC AACTTCGAAG GCCGTATCAA
TCCAGACGTT AAAATGAACT ACCTTGCGTC GCCACCATTG GTAGTGGCCT ATGCATTGGC CGGAACAATG GATTTTGATT
TTGAAAAGCA GCCCCTTGGG AAGGACAAGG ATGGCAATGA TGTTTATTTG AAGGATATTT GGCCTAGCCA GAAAGATGTG
AGCGACACAA TCGCTTCCGC GATCAACAGC GAGATGTTCA CAAAGAACTA TGCCGATGTA TTCAAAGGAG ATGAACGCTG
GCGTAACTTA CCTACCCCTA GTGGGAATAC ATTTGAATGG TCTCCGGATA GCACTTATGT TCGTAAACCC CCATACTTTG
AGGGAATGCC GGCCGAACCT GAACCGGTAG CGGACATCTC CGGCGCTCGC GTCCTGGCCT TGCTGGGAGA TTCTGTAACA
ACCGATCACA TTTCTCCAGC GGGGAGCATC AAACCTGGGA CTCCGGCAGC GCAGTATTTG GATGAACACG GCGTTGATCG
TAAAGACTAC AACAGTTTTG GTTCACGTCG TGGGAACCAT GAGGTGATGA TTCGTGGCAC GTTCGCAAAT ATTCGTTTAC
GCAACCTTTT ATTGGACGAT GTAGCAGGTG GCTACACACG CGATTTTACG CAAGATGGAG GTCCCCAGGC CTTTATTTAT
GATGCTGCTC AGAATTATGC CGCGCAGAAC ATTCCGCTGG TGGTGCTGGG GGGAAAGGAA TATGGCTCAG GCAGTAGCCG
CGACTGGGCG GCAAAAGGTA CGCGCCTGCT TGGCGTCCGT GCAGTAATTG CTGAGTCCTT TGAGCGCATC CATCGTTCCA
ACTTAATCGG TATGGGTGTT ATCCCTCTTC AATTCCCTGA CGGGAAGTCC GCCAAGGATC TTGGACTGGA CGGAACGGAG
GTATTCGACA TCACTGGCAT TGAAGAGCTG AATAAAGGGA AAACACCTAA AACGGTGCAT GTGAAAGCAT CGAAAAATGG
AAGCGACGCG GTGGAGTTTG ACGCCGTGGT TCGCATTGAC ACGCCGGGCG AGGCGGATTA CTACCGTAAC GGCGGTATCC
TTCAATACGT GTTGCGCAAT ATGCTGAAGT CTGGCCGCCT TCAGGGGATG TCTCGCCTGA GCTTTGTGTG CCGTCTGCTG
GCTGCAACCG CCTTTGCTGT GGCCCTGTTG CTTGGGTTGG GTGATGTTCC GCGCGCAGCG GCCACAGATG ATCGCCTTCA
GTTTACAGCC ACTACCTTAT CAGGCGCTCC CTTCAATGGT GCTAGTCTTC AGGGCAAGCC AGCTGTACTT TGGTTCTGGA
CCCCCTGGTG TCCGTACTGC AATGCTGAAG CTCCCGGAGT CAGCCGCGTC GCCGCAGCCA ACCCGGGAGT AACATTCGTC
GGTGTTGCAG CGCACTCCGA GGTGGGAGCT ATGGCTAATT TCGTAAGCAA ATATAACTTA AACTTTACTA CGTTGAACGA
TGCTGACGGC GCGATCTGGG CCCGTTATGG CGTTCCGTGG CAACCTGCCT ATGTTTTTTA CCGTGCAGAT GGTTCTAGTA
CTTTTGTAAA TAA
The non-MAP nucleotides are in italics.
>MAP1201 c+2942c fusion protein (SEQ ID NO: 94):
SRRSDELGEFVLDHGAVVIAAVTSCTNTSNPEVMLGAALLARNAVEKGLASKPWVKTTMAPGSQVV
HDYYDKAGLWPYLEKLGFYLVGYGCTTCIGNSGPLPEEISKAINDNDLSVTAVLSGNRMFEGRINP
DVKMNYLASPPLVVAYALAGTMDFDFEKQPLGKDKDGNDVYLKDIWPSQKDVSDTIASAINSEMFT
KNYADVFKGDERWRNLPTPSGNTFEWSPDSTYVRKPPYFEGMPAEPEPVADISGARVLALLGDSVT
TDKISPAGSIKPGTPAAQYLDEHGVDRKDYNSFGSRRGNHEVMIRGTFANIRLRNLLLDDVAGGYT
RDFTQDGGPQAFIYDAAQNYAAQNIPLVVLGGKEYGSGSSRDWAAKGTRLLGVRAVIAESPERIHR
SNLIGMGVIPLQFPDGKSAKDLGLDGTEVFDITGIEELNKGKTPKTVHVKASKNGSDAVEFDAVVR
IDTPGEADYYRNGGILQYYLRNMLKSGRLQGMSRLSFVCRLLAATAFAVALLLGLGDVPRAAATDD
RLQFTATTLSGAPFNGASLQGKPAVLWFWTPWCPYCNAEAPGVSRVAAANPGVTFVGVAAHSEVGA
MANFVSKYNLNFTTLNDADGAIWARYGVPWQPAYVFYRADGSSTFVN
The non-MAP amino acids are in italics. The portion of MAP1201c
is in black. The bold region is MAP2942c.
>MAP2121c nucleic acid (SEQ ID NO: 95):
ATGACGTCGGCTCAAAATGAGTCTCAAGCACTTGGTGATCTGGCTGCCAGGCAACTCGCCAACGCAACCAAGA
CCGTCCCCCAGCTCTCGACGATCACGCCGCGCTGGCTGCTGCACCTGCTGAACTGGGTTCCGGTGGAGGCGGG
CATCTACCGGGTGAACCGGGTGGTCAATCCCGAGCAGGTCGCCATCAAGGCCGAGGCCGGCGCCGGCAGTGAA
GAGCCGCTACCGCAGACCTATGTGGACTACGAGACCAGCCCGCGCGAGTACACGCTGCGCAGCATTTCCACGC
TGGTCGACATCCACACCCGGGTCTCCGACCTGTACTCGAGCCCGCACGATCAGATCGCCCAGCAGCTGCGGCT
GACCATCGAGACCATCAAGGAGCGCCAGGAGCTGGAGCTGATCAACAGCCCCGAGTATGGGCTGCTGGCCCAG
GCGACGCCGGAGCAGACGATCCAGACGCTGGCCGGGGCTCCCACGCCCGACGACCTCGACGCGCTGATCACCA
AGGTGTGGAAGACGCCCAGTTTCTTCCTGACCCACCCGCTGGGCATCGCGGCGTTCGGGCGCGAGGCCACCTA
CCGGGGGGTGCCGCCGCCGGTGGTGAGCCTGTTCGGCGCCCAGTTCATCACCTGGCGCGGTATTCCGCTGATC
CCGTCGGAGAAGGTGCCGGTGGAGGACGGCAAGACGAAGTTCATCCTGGTCCGCACCGGCGAGGAACGTCAGG
GCGTCGTCGGGCTGTTCCAGCCCGGCCTGGTCGGGGAGCAGGCGCCGGGGCTGTCGGTGCGGTTCACCGGCAT
CAACCAGTCGGCGATCGCGACCTACCTGGTCACGCTGTACACCTCCCTGGCCGTCCTGACCGATGACGCGCTC
GCCGTGCTCGACGACGTCGCGGTGGATCAGTTCCATGAGTACAAGTGA
>MAP2121c protein (SEQ ID NO: 96):
MTSAQNESQALGDLAARQLANATKTVPQLSTITPRWLLHLLNWVPVEAGIYRVNRVVNPEQVAIKAEAGAGSE
EPLPQTYVDYETSPREYTLRSISTLVDIHTRVSDLYSSPHDQIAQQLRLTIETIKERQELELINSPEYGLLAQ
ATPEQTIQTLAGAPTPDDLDALITKVWKTPSFFLTHPLGIAAFGREATYRGVPPPVVSLFGAQFITWRGIPLI
PSDKVPVEDGKTKFILVRTGEERQGVVGLFQPGLVGEQAPGLSVRFTGINQSAIATYLVTLYTSLAVLTDDAL
AVLDDVAVDQFHEYK*
>MAP1201c nucleic acid (SEQ ID NO: 97):
atgttaaagttggcaccgtcgccgacacggcccgtcggcggaagaactaaatccttggga
gttgaagtgactgattctgtgaactcgttcggagcccgcaacaccctcaaggtcggcgac
aagagttaccagatctatcgcctcgacgccgtccccaataccgagaagcttccctacagc
ctcaaggtgctggccgagaacctgctgcgcaacgaggacggcagcaacatcaccaaagac
cacatcgaggccatcgcgaactgggatcccaaggcggagcccagcattgaaatccagtac
acgcccgcccgggtggtgatgcaggacttcaccggggtgccgtgcatcgtcgacctggcc
accatgcgggaggcgatcgccgacctgggcggcaacccggagaaggtcaacccgctcgcg
acgcccgcccgggtggtgatgcaggacttcaccggggtgccgtgcatcgtcgacctggcc
accatgcgggaggcgatcgccgacctgggcggcaacccggagaaggtcaacccgctcgcg
ccggccgacctggtgatcgaccactcggtgatcgccgacctgttcggcacggccgacacg
ttcgagcgcaacgtcgagatcgaataccagcgcaacggcgagcgctaccagttcctgcgc
tgggggcagggcgccttctccgacttcaaggtggtgccgccgggcaccgggatcgtgcac
caggtgaacatcgagtacctggcccgggtggtgatggagcgcgacggggtggcctatccg
gacacctgcgtgggcaccgactcgcacaccacgatggtcaacggcctgggcgtgctgggc
tggggcgtcggcggcatcgaggccgaggccgcgatgctcggccagccggtgtcgatgctg
atcccgcgggtggtcggcttcaagctgaccggtgagatccagccgggcgtgaccgccacc
gacgtggtgctgaccgtcaccgagatgctgcgcaagcacggcgtggtcggcaagttcgtc
gagttctacggcgagggggtggccgaggtgccgctggccaaccgcgccaccctgggcaac
atgagccccgaattcggttccaccgcagcgattttcccgatcgacgaggaaaccatcgac
tacctgaagttcaccggccgcaacgccgagcaggtggcgctggtcgagacctacgccaaa
gagcagggcctgtggcacgaccccgcccacgagccggccttctcggagtacctggagctc
gacctgtcccaggtggtgccctcgatcgccgggcccaagcgcccccaggaccgaattgcg
ttgtcgcaggccaagtccgtcttccgcgagcagatccccagctacgtcggcgacggcgac
gggcagcagggctactcgaagctggacgaggtggtcgacgagacgttcccggccagcgac
ccgggggcgccgtccaacggccacgccgacgacctgcccgcggtgcagtcggccgccgcg
cacgccaacggccgcccgagcaacccggtgacggtccgctccgacgagctgggcgagttc
gtgctcgaccacggcgcggtggtgatcgccgcggtcacgtcgtgcaccaacacctccaac
cccgaggtgatgctgggcgcggcgctgttggcgcgcaacgccgttgagaaggggctggcc
tccaagccgtgggtgaagaccacgatggcgccgggctcgcaggtggtccacgactactac
gacaaggccgggctgtggccgtatttggagaagctcggcttctatctggtcggctacggc
tgcaccacctgcatcggcaactccggtccgctgcccgaggagatctcgaaggccatcaac
gacaacgacctgtcggtgaccgcggtgctctcgggtaaccgcaacttcgagggccgcatc
aacccggacgtgaagatgaactacctggcgtcgccgccgctggtggtggcctacgcgctg
gccggcaccatggacttcgacttcgaaaagcagccgctgggcaaggacaaggacggcaac
gacgtctacctgaaggacatctggccgtcgcagaaggacgtctcggacaccatcgcatcg
gcgatcaactccgagatgttcaccaagaactacgccgacgtcttcaagggtgacgagcgc
tggcgcaacctgcccaccccgagcggcaatacctttgagtggagcccggattcgacgtac
gtgcgcaagccgccgtacttcgagggcatgccggccgagcccgagccggtcgccgacatc
tccggcgcgcgggtgctggcgctgctgggcgactcggtgaccaccgaccacatctccccc
gccggcagcatcaagccgggcaccccggcggcgcagtacctcgacgagcacggcgtggac
cgcaaggattacaactccttcggctcgcggcgcggcaaccatgaggtgatgatccgcggc
acgttcgccaacatccggctgcgcaacctgctgctcgacgacgtggccggcggctacacc
cgcgacttcacccaggacggcggtccgcaggcgttcatctacgacgcggcgcaaaactat
gcggcacaaaacattccgctggtggtgctgggcggcaaggaatacgggtccggctcgtcg
cgagactgggcggccaagggcacccggctgctgggcgtacgcgcggtgatcgccgagtcg
ttcgaacggatccaccgctccaacctgatcgggatgggtgtgatcccgctgcagttcccg
gacggcaaatcggccaaggacctggggctggacggcaccgaggtgttcgacatcaccggc
atcgaagagctcaacaagggcaagacaccgaagacggtgcacgtcaaggcgagcaaaaac
ggttcggacgcagtcgaattcgatgcggtggtgcgcatcgacacccccggtgaggcggac
tactaccgcaacggcggcatcctgcagtacgtgctgcgcaacatgctcaagtccggctga
>MAP1201c protein (SEQ ID NO: 98):
MLKLAPSPTRPVGGRTKSLGVEVTDSVNSFGARNTLKVGDKSYQIYRLDAVPNTEKLPYS
LKVLAENLLRNEDGSNITKDHIEAIANWDPKAEPSIEIQYTPARVVMQDFTGVPCIVDLA
TMREAIADLGGNPEKVNPLAPADLVIDHSVIADLFGTADTFERNVEIEYQRNGERYQFLR
WGQGAFSDFKVVPPGTGIVHQVNIEYLARVVMERDGVAYPDTCVGTDSHTTMVNGLGVLG
WGVGGIEAEAAMLGQPVSMLIPRVVGFKLTGEIQPGVTATDVVLTVTEMLRKHGVVGKFV
EFYGEGVAEVPLANRATLGNMSPEFGSTAAIFPIDEETIDYLKFTGRNAEQVALVETYAK
EQGLWHDPAHEPAFSEYLELDLSQVVPSIAGPKRPQDRIALSQAKSVFREQIPSYVGDGD
GQQGYSKLDEVVDETFPASDPGAPSNGHADDLPAVQSAAAHANGRPSNPVTVRSDELGEF
VLDHGAVVIAAVTSCTNTSNPEVMLGAALLARNAVEKGLASKPWVKTTMAPGSQVVHDYY
DKAGLWPYLEKLGFYLVGYGCTTCIGNSGPLPEEISKAINDNDLSVTAVLSGNRNFEGRI
NPDVKMNYLASPPLVVAYALAGTMDFDFEKQPLGKDKDGNDVYLKDIWPSQKDVSDTIAS
AINSEMFTKNYADVFKGDERWRNLPTPSGNTFEWSPDSTYVRKPPYFEGMPAEPEPVADI
SGARVLALLGDSVTTDHISPAGSIKPGTPAAQYLDEHGVDRKDYNSFGSRRGNHEVMIRG
TFANIRLRNLLLDDVAGGYTRDFTQDGGPQAFIYDAAQNYAAQNIPLVVLGGKEYGSGSS
RDWAAKGTRLLGVRAVIAESFERIHRSNLIGMGVIPLQFPDGKSAKDLGLDGTEVFDITG
IEELNKGKTPKTVHVKASKNGSDAVEFDAVVRIDTPGEADYYRNGGILQYVLRNMLKSG
>MAP2942c nucleic acid (SEQ ID NO: 99):
gtgcgtcttcagggcatgtcccgtttgtcatttgtctgcaggcttttggccgcaaccgct
ttcgccgtcgccctgctactcgggctgggcgacgtgccgcgcgcggcggccaccgacgac
cgcctgcaattcaccgcgaccacgctcagcggcgcgccgttcaacggcgccagtctgcag
ggcaagcccgccgtgctgtggttctggacgccgtggtgcccgtactgcaacgccgaggcc
ccgggcgtgagccgggbggccgccgccaacccgggcgtcaccttcgtcggcgtcgccgcc
cactccgaagtcggcgccatggccaacttcgtctccaagtacaacctgaacttcaccacg
ctcaacgacgccgacggcgcgatctgggcccgctacggcgtgccctggcagcccgcgtac
gtgttctaccgggcggacggcagctccaccttcgtcaacaaccccacctcggcgatgccc
caggacgaactggccgcccgggtggcggcgctgcgctga
>MAP2942c protein (SEQ ID NO: 100):
VRLQGMSRLSFVCRLLAATAFAVALLLGLGDVPRAAATDDRLQFTATTLSGAPFNGASLQ
GKPAVLWFWTPWCPYCNAEAPGVSRVAAANPGVTFVGVAAHSEVGAMANFVSKYNLNFTT
LNDADGAIWAJRYGVPWQPAYVFYRADGSSTFVNNPTSAMPQDELAARVAALR

### Example 4: Using a Peptide Array to Further Define the Precise Epitopes that React to Antibodies from Infected Cows

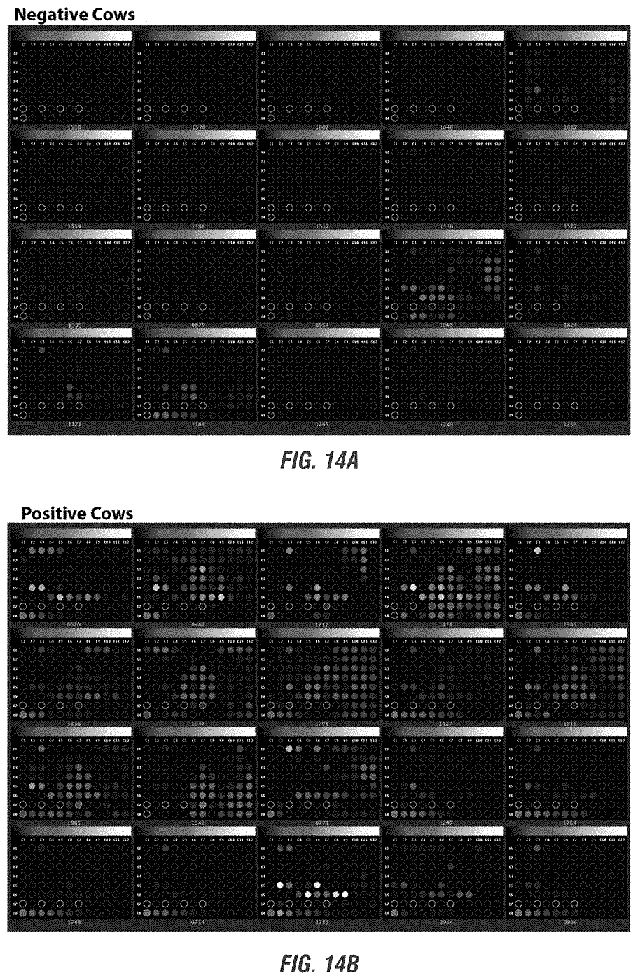

Peptide arrays for MAP1596, MAP2609, and MAP2942c were commercially obtained in order to identify immunodominant epitopes. A total of 72 peptides are present on the MAP1596 peptide array. They are each 15 amino acids in length with 10 amino acid overlaps. Serum samples from 20 negative and 20 positive cows were analyzed on the MAP1596 peptide array. These same sera samples were also used in Example 2 and each were diluted 1:300. Detailed methods for how the arrays were processed are well known and routine in the art. The normalized peptide arrays from 20 positive cows and 20 negative cows are shown in FIG. 14. The results suggest that the most immunogenic peptides of MAP1596 are the overlapping peptides in E3 and E4 as well as the peptide in A3.

The above disclosure is intended to be illustrative and not exhaustive. This description will suggest many variations and alternatives to one of ordinary skill in this art. All these alternatives and variations are intended to be included within the scope of the claims. Those familiar with the art may recognize other equivalents to the specific embodiments described herein which equivalents are also intended to be encompassed by the claims.

1. A method of determining whether an animal is infected with Mycobacterium avium subspecies paratuberculosis (MAP), the method comprising:
(a) obtaining a sample from the animal; and
(b) detecting the presence or absence of the binding of a biomarker in the sample with one or more MAP derived antigens.

2. The method of claim 1, wherein said antigens are MAP1272c, MAP1569, MAP2121c, MAP2942c, MAP2609, and/or MAP1201c+2942c.

3. The method of claim 2, wherein said antigens are MAP1272c, MAP1569, MAP2942c, and MAP2609.

4. The method of claim 2, wherein said antigens are MAP1272c, MAP1569, MAP2121c, MAP2942c, MAP2609, and MAP1201c+2942c.

5. The method of claim 1, wherein said antigens are MAP0019c, MAP0117, MAP0123, MAP0357, MAP0433c, MAP0616c, MAP0646c, MAP0858, MAP0953, MAP1152, MAP1224c, MAP1298, MAP1506, MAP1525, MAP1561c, MAP1651c, MAP1761c, MAP1782c, MAP1960, MAP1968c, MAP1986, MAP2093c, MAP2100, MAP2117c, MAP2158, MAP2187c, MAP2195, MAP2288c, MAP2447c, MAP2497c, MAP2694, MAP2875, MAP3039c, MAP3305c, MAP3527, MAP3531c, MAP3540c, MAP3762c, MAP3773c, MAP3852c, MAP4074, MAP4143, MAP4225c, MAP4231, and/or MAP4339.

6. The method of claim 1, wherein said antigen comprises one or more immunogenic fragments of MAP1569, MAP2609, and/or MAP2942c.

7. The method of claim 1, wherein the biomarker is an antibody indicative of infection with MAP.

8. The method of claim 1, wherein said antigens are able to detect subclinical infections with MAP.

9. The method of claim 1, wherein the sample is serum or milk.

10. The method of claim 1, wherein the animal is a ruminant.

11. The method of claim 10, wherein the ruminant is a cow.

12. The method of claim 1, wherein the detecting is accomplished by ELISA.

13. The method of claim 1, wherein the detecting is accomplished by a multiplex bead-based immunoassay.

14. The method of claim 1, wherein the detecting is accomplished by flow cytometry.

15. The method of claim 1, further comprising:
(c) treating the animal to kill or deactivate MAP bacteria to ameliorate the symptoms of or prevent the onset of Johne's disease if the presence of the biomarker is detected in the sample.

16. A method of detecting antibodies which are associated with Mycobacterium avium subspecies paratuberculosis (MAP) in a biological sample, the method comprising:
(a) contacting the sample with one or more MAP derived antigens; and
(b) detecting the binding the antigens with an antibody in the sample.

17. The method of claim 16, wherein said antigens are MAP0019c, MAP0117, MAP0123, MAP0357, MAP0433c, MAP0616c, MAP0646c, MAP0858, MAP0953, MAP1152, MAP1224c, MAP1298, MAP1506, MAP1525, MAP1561c, MAP1651c, MAP1761c, MAP1782c, MAP1960, MAP1968c, MAP1986, MAP2093c, MAP2100, MAP2117c, MAP2158, MAP2187c, MAP2195, MAP2288c, MAP2447c, MAP2497c, MAP2694, MAP2875, MAP3039c, MAP3305c, MAP3527, MAP3531c, MAP3540c, MAP3762c, MAP3773c, MAP3852c, MAP4074, MAP4143, MAP4225c, MAP4231, and/or MAP4339.

18. The method of claim 16, wherein the sample is serum or milk.

19. The method of claim 16, wherein the detecting is accomplished by ELISA.

20. The method of claim 16, wherein the detecting is accomplished by a multiplex bead-based immunoassay.

21. The method of claim 16, wherein the detecting is accomplished by flow cytometry.

22. A method of diagnosing and treating Johne's disease, the method comprising:
(a) obtaining a sample from an animal;
(b) detecting the presence or absence of the binding of a biomarker in the sample with one or more MAP derived antigens; and
(c) treating an animal with the presence of said biomarker to kill or deactivate MAP bacteria to ameliorate the symptoms of or prevent the onset of Johne's disease.

23. The method of claim 22, wherein said antigens are MAP1272c, MAP1569, MAP2121c, MAP2942c, MAP2609, and/or MAP1201c+2942c.

24. The method of claim 22, wherein said antigens are MAP0019c, MAP0117, MAP0123, MAP0357, MAP0433c, MAP0616c, MAP0646c, MAP0858, MAP0953, MAP1152, MAP1224c, MAP1298, MAP1506, MAP1525, MAP1561c, MAP1651c, MAP1761c, MAP1782c, MAP1960, MAP1968c, MAP1986, MAP2093c, MAP2100, MAP2117c, MAP2158, MAP2187c, MAP2195, MAP2288c, MAP2447c, MAP2497c, MAP2694, MAP2875, MAP3039c, MAP3305c, MAP3527, MAP3531c, MAP3540c, MAP3762c, MAP3773c, MAP3852c, MAP4074, MAP4143, MAP4225c, MAP4231, and/or MAP4339.

25. The method of claim 22, wherein the biomarker is an antibody indicative of infection with MAP.

26. The method of claim 22, wherein said antigens are able to detect subclinical infections with MAP.

27. The method of claim 22, wherein the sample is serum or milk.

28. The method of claim 22, wherein the animal is a ruminant.

29. The method of claim 28, wherein the ruminant is a cow.

30. The method of claim 22, wherein the detecting is accomplished by ELISA.

31. The method of claim 22, wherein the detecting is accomplished by a multiplex bead-based immunoassay.

32. The method of claim 22, wherein the detecting is accomplished by flow cytometry.

33. A kit for determining the presence or absence of a biomarker in a sample, the kit comprising:
a composition comprising one or more MAP derived antigens, and means for detecting the binding of said antigen with a biomarker present within a sample.

34. The kit of claim 33, wherein said antigens are MAP1272c, MAP1569, MAP2942c, and MAP2609.

35. The kit of claim 33, wherein said antigens are MAP1272c, MAP1569, MAP2121c, MAP2942c, MAP2609, and MAP1201c+2942c.

36. The kit of claim 33, wherein said antigens are MAP0019c, MAP0117, MAP0123, MAP0357, MAP0433c, MAP0616c, MAP0646c, MAP0858, MAP0953, MAP1152, MAP1224c, MAP1298, MAP1506, MAP1525, MAP1561c, MAP1651c, MAP1761c, MAP1782c, MAP1960, MAP1968c, MAP1986, MAP2093c, MAP2100, MAP2117c, MAP2158, MAP2187c, MAP2195, MAP2288c, MAP2447c, MAP2497c, MAP2694, MAP2875, MAP3039c, MAP3305c, MAP3527, MAP3531c, MAP3540c, MAP3762c, MAP3773c, MAP3852c, MAP4074, MAP4143, MAP4225c, MAP4231, and/or MAP4339.

37. The kit of claim 33, wherein the biomarker is an antibody indicative of infection with MAP.


SEQUENCE LISTING (106 sequences)

SEQ ID NO: 1 - protein, 492 aa, Mycobacterium avium-paratuberculosis
MNASLRRISVTVMALIVLLLLNATMTQVFAADSLRADPRNQRVLLDEYSRQRGQIVAGGQLLAYSVATDNRFRFLRVYPNPAQYAPVTGFYSLRYSSTGLERAEDPLLNGSDERLFGRRLADFFTGRDPRGANVDTTIRPRVQQAAWDGMQQGCGGPPCKGAVVALEPSTGKILAMVSSPSYDPNLLSSHDPEVQAQAWQRLRDDPDNPMTNRAISETYPPGSTFKVITTAAALQAGASDTEQLTAAPSIPLPNSTATLENYGGQACGNDPTVSLQQAFALSCNTAFVQLGILTGADALRSMARSFGLDSTPSVIPLQVAESTIGIIPDAAALGMSSIGQKDVALTPLQNAEIAATIANGGVTMQPYLVDSLKGPDLTTISTTTPYEQRRAVSPQVAAKLTELMVGAEKVAQQKGAIPGVQIASKTGTAEHGSDPRHTPPHAWYIAFAPAQTPKVAVAVLVENGADRLSATGGALAAPIGRAVIEAALQGGP

SEQ ID NO: 2 - protein, 407 aa, Mycobacterium avium-paratuberculosis
LVSVALRTDQGFIPAVFRACSPPLTCSYSQPLSTASGRQPWEGPTQMIEIVPGHRALLGGMVAGLIGLAVAAGGTASADPLPPAPAPVPAPAPANLGPELVPPSRYLAAPQATTAATQVTPATPGTPGPAPAPAPAPAPAPAPATSGTIREFLQSKGVKFEAQKPQGFKALDITLPMPARWTQVPDPNVPDAFAVIADRHGSSIYSSNAQVVVYKLVGNFDPREAITHGYVDSQKLPAWQPTNASMADFGGFPSSIVEGTYRDGDLTLNTSRRHVIATSGPDKYLVSLAVTTDRAVAVADAPATDAIVNGFRVTVPGASAPAPTAAPVALPAQAPAVAPVAPAPVAPAAPTAPAPAAAAPLVPLAQTAPAAPAGLPAQPLPNQQHTPSLLAMVPGLPPLPNFSFLQH

SEQ ID NO: 3 - protein, 74 aa, Mycobacterium avium-paratuberculosis
MLSTIRKVLDYQLTIAELLGLGILLGTPYLIVGVIWSSTHTAHLHDMHGVDLVVSFLGSIVSWPVLLFANVCMT

SEQ ID NO: 4 - protein, 301 aa, Mycobacterium avium-paratuberculosis
MTAPVWMALPPEVHSTLLSSGPGPGPLLAAAATWTGLSTQYDSAATELTAVLTGSMPVWDGPTADRYVAAHMPYLAWLQLAGALSAEAAAQHQGVATAYTAALAAMPTLPELAAMPTLPELAANHATHAALVATNFFGVNTIPIAVNEADYARMWTQAATTMTTYQATTEAVQMSSVAGSGTGGRPAAAAGPERERARGPERAPALGPEPAPVREPEVAPAVGPAPAAAAVRVPFSCPPQKRSGRCCSGPTVSRSPVRASRTGARRSTCRISGISSTATLRPWPGSSRTFRACSTRPSSRR

SEQ ID NO: 5 - protein, 336 aa, Mycobacterium avium-paratuberculosis
VPNRRRRKLSTAMSAVAALAVASPCAYFLVYESTAGNKAPEHHEFKQAAVMSDLPGELMGALSQGLSQFGINLPPVPALSGGATSTPGLASPGLGSPGLGTPGLGTPGLTNPGLTSPGATSPGLTSPGLTSPGLTSPGLTSPGAAPTTPGLTAPGALPTTPGGGVATPGAGLNPALSNPGLTSPAGTAPGLGSPTVAPSEVPIDSGAGLDPGAGGTYPILGDPSTFGNASPIGGGGTGLGGGSSSGGSGGLVNDVMQAANQLGAGQAIDLLKGLVMPAITQGMHGGAAAGALPGAAGALPGAAGALPGAAGALPGAAGAAGALPAAAGAAPALPPV

SEQ ID NO: 6 - protein, 350 aa, Mycobacterium avium-paratuberculosis
VTSADQLEGLDLAALDSYLRSLGIGRDGELRAEFISGGRSNLTFRVYDDATSWLVRRPPLHGLTPSAHDMAREYRVVAALQDTPVPVARTIGLCEDESVLGAPFQIVEFVAGQVVRRRAQLESFSHTVIEGCVDSLIRVLVDLHSVDPDAVGLADFGKPSGYLERQVRRWGSQWALVRLPEDRRDADVERLHSGLGQAIPQQSRTSIVHGDYRIDNTILDADDPTKVRAVVDWELSTLGDPLSDAALMCVYRDPALDLIVNAQAAWTSPLLPTADELADRYSLVAGIPLAHWEFYMALAYFKLAIIAAGIDFRRRMSDQARGLGDAAEHTPEVVAPLISRGLAELAKLPG

SEQ ID NO: 7 - protein, 515 aa, Mycobacterium avium-paratuberculosis
VSPNRRAVAEFAEFIAAIDQGTTSTRCMIFDHQGAEVARHQLEHEQILPRAGWVEHDPIEIWERTSSVLTSVLNRANLSAENLAALGITNQRETTLVWNRKTGRPYYNAIVWQDTRTDRIASALDRDGRGQVIRRKAGLPPATYFSGAKLQWILDNVDGVREAAERGDALFGTADSWVLWQLTGGPRGGVHATDVTNASRTMLMDLETLDWDDELLSFFTIPRAMLPEIGPSSSPRPFGVTSDTGPAGGRIPITAVLGDQHAAMVGQVCLAEGEAKNTYGTGNFLLLNTGESIVRSEHGLLTTVCYQFGDAKPVYALEGSIAVTGAAVQWLRDQLGIISGAAQSESLARQVDDNGGVYFVPAFSGLFAPYWRSDARGAIVGLSRFNTNAHLARATLEAICYQSRDVVDAMAADSGVRLEVLKVDGGITGNDLCMQIQADVLGVDVVRPVVAETTALGAAYAAGLAVGFWADPGELRANWREDKRWTPAWSDEQRTAGYAGWHKAVQRTLDWADVT

SEQ ID NO: 8 - protein, 289 aa, Mycobacterium avium-paratuberculosis
MSEVVTGDAVVLDVQIAQLPVRALSALIDIAVIVVGYLLGLMLWAATLTQFDTALSNAILLIFTVLVIVGYPLILETATRGRSVGKIALGLRVVSDDGGPERFRQALFRALASLVEIWMLFGSPAVICSILSPKAKRIGDIFAGTVVVNERGPRLGPPPAMPPSLAWWASSLQLSGLSSGQAEVARQFLSRAAQLDPGLRLQMAYRIAGDVVARIAPPPPGAPPELVLAAVLAERHRRELARLRPPAPWPAPGYPPAWPGSGPAPQWPAPGPANPGPPEGFSAGFTPPR

SEQ ID NO: 9 - protein, 330 aa, Mycobacterium avium-paratuberculosis
VDVDAFVLAHRPTWDRLDRLVGRRRSLSGAEIDELVELYQRVSTHLSMLRSASSDSMLVGRLSSLVARARSAVTAAHAPLSSTFVRFWTVSFPVVAYRSWRWWVATGAAFFAVVVIVALWVAGNPEVQSALGTPSDIDQLVNHDVESYYSEHPAAAFALQIWVNNSWVSAQCIALSVVLGLPIPLVLFENAANLGVIAGLMFPAGKGGLLLGLLAPHGLLELTAVFLAGATGMRLGWSVISPGDRPRGQVLAEQGRAVVSVAVGLVAVLLVSGLIEALVTPSPLPTFVRVGIGVVAEAAFLCYIGYFGRRGVKAGESGDIEEAPDVVPAG

SEQ ID NO: 10 - protein, 380 aa, Mycobacterium avium-paratuberculosis
MSTTPKQLDMAAILADTTNRVVVCCGAGGVGKTTTAAAIALRAAEYGRNVCVLTIDPAKRLAQALGVNDLGNTPQRVPLAAEVPGELHAMMLDMRRTFDEMVVQYSGPGRAQAILDNQFYQTVASSLAGTQEYMAMEKLGQLLAEDRWDLVVVDTPPSRNALDFLDAPKRLGSFMDSRLWRLLLAPGRGIGRLVTGAMGLAMKAMSTILGSQMLADAAAFVQSLDATFGGFREKADRTYALLKRRGTQFVVVSAAEPDALREASFFVDRLSQEGMPLAGLVLNRTHPPLCSLPAERAIDGTEMLEHDGDPETTSLAAAVLRIHADRAQTAKREIRLLSRFTGANPHVPVIGVPSLPFDVSDLEALRALADQITSNQATAR

SEQ ID NO: 11 - protein, 438 aa, Mycobacterium avium-paratuberculosis
VGRLLFSNCGDTSGQRAESAAPMTEISASRGPVARGSMARVGTATAVTALCGYAVIYLAARDLAPGGFSVFGVFWGAFGLVTGAANGLLQETTREVRVMPYLEVAPVKRTHPLRVAMLLGAAAAVVIAGSSPLWSGRVFVEARPLSVLLLSVGLAGFCVHATLLGMLAGTNEWTRYGALMVTDAVIRVMVAAATVVLGWRLVGFLWATVAGAVAWLILLAASPATRATARLLTPGGTATFLRGAAHSITAAGASAILVMGFPVLLKLTSAELGAQGGVIILAVTLTRAPLLVPLTAMQGNLIAHFVDERSDRVRALIGPAAIVGAIGAVGVLAAGVLGPWVLRVVFGPQYQAGSALLAWLTAAAVAIAMLTLTGAAAVAAALHRAYALGWVGATVASGLLLALPLSLQTRTVVGLLCGPLVGIGVHLVALSRAARLTG

SEQ ID NO: 12 - protein, 362 aa, Mycobacterium avium-paratuberculosis
MASETTMDFDPSPTQQAVADVVTSVLDRELSWEALVDGGVTALPVPERLGGDGVGLPEVATVLTEVGRRGAITPALATLGFAVLPLLELASEEQQDRFLAGVARGGVLTAALNEPGTPLPDRPATTFADGRLSGTKIGVGYAAQADWMIVTADSAVVVVSPKADGVQVVQTPTSNGSDEYTVSFTGVAVADSDVLAGATAARVNQLALAAVGAYADGLVSGALRLTADYVANRKQFGKPLSTFQTVAAQLAEVYIASRTIDLVAKSVVWGLSEGRDVDHDLGVLGYWVASQAPPAMQLCHHLHGGMGMDITYPMHRYYSTIKDLTRLLGGPSHRLDLVAIASAAQPGAAGRHADDLVGAQCS

SEQ ID NO: 13 - protein, 384 aa, Mycobacterium avium-paratuberculosis
MSRMWLRAGGLATGSMLLAGCQFGGLNSLAMPGTAGHGSGAYSITVELPDVATLPQNSPVMVDDVTVGSVAGISAEQRSDGSFYAAVKLALDKNVVLPANSTATVAQTSLLGSMHIDLNRPKDRPAVGRLTDGSKIAEANTGRYPTTEEVLSALGVVVNKGNVGALEEITDETYRAVAGRQDQFVDLVPRLAELTSGLNRQVNDIIDAVDGLNRFSASLARDKDNLGRALDTLPEAIRVLNKNRDHIVEAFSALHKLADVTSHILAKTKVDFAADLKDLYAAVKALNDNRRNFVTSLQLLLTFPFPNFGIKQAVRGDYLNVFTTFDLTLRRLGETFFTTAYFDPNMAHMNEILNPPDFLVGEMANLSGQAADPFKIPPGTASGQ

SEQ ID NO: 14 - protein, 215 aa, Mycobacterium avium-paratuberculosis
VSSDALVTITSDAGGETGQPPRNRRQEETFRKVLAAGIETLREKSYSDLTVRAVAARAKVAPATAYTYFSSKNHLIAEVYLDLVRQVPYFTDVNDPMPTRVEQVLRHLALVVADEPEVSAACTTALLSGGADPAVRAARDRIGVEIHRRITSAMGPDADPTTVSALEMSFFGALVQAGSGEFSYREIADRLAYVVRLILTGTTQASPETEAGDTR

SEQ ID NO: 15 - protein, 148 aa, Mycobacterium avium-paratuberculosis
MAPLITLVVGSLVAWVVGRLGVAYVDGWAPALAVGLAAMFVLTGIAHFAPPLRADLVAIVPPRLPAPGLLVSLTGVLELLGALGLLLPATRAAAAGCLLVLMLAMFPANIHASRMPDPPKSMTTRLPLRIGMEIVFLAAAVAVALGGR

SEQ ID NO: 16 - protein, 156 aa, Mycobacterium avium-paratuberculosis
LPSSNTTTQPDLVDVRGPRFAAWVTTAVLVLALAVSAVSPAAAAVILAVQAVVFAIGAVGGPRKHPYGRVFAAVVAPRLGPVREREPIPPLKFAQLVGLIFAVLGAAGFAAGASLFGLVATAAALAAAFLNAAFGICLGCQLYPLVARFRRPARST

SEQ ID NO: 17 - protein, 233 aa, Mycobacterium avium-paratuberculosis
MDTAASSPRVLVVDDDSDVLASLERGLRLSGFEVSTAVDGAEALRSATETRPDAIVLDINMPVLDGVSVVTALRAMDNDVPVCVLSARSSVDDRVAGLEAGADDYLVKPFVLAELVARVKALLRRRGATATSSSETITVGPLEVDIPGRRARVNGVDVDLTKREFDLLAVLAEHKTAVLSRAQLLELVWGYDFAADTNVVDVFIGYLRRKLEANGGPRLLHTVRGVGFVLRMQ

SEQ ID NO: 18 - protein, 182 aa, Mycobacterium avium-paratuberculosis
VRWTRRKPRSQTLTFAIEARCRECHYKATERAKVTTYPAERVADQLRPTPPAVPSKFGGLWILAVVSASNSSTPAISPSAKCSRSAAVCQSSSTAPCIRLRSSRPSWSRADCSLAPLTSHSAPGYRAVHDRSSYSAVCGTNAKALPVVRMKSSKFVLRSSVFAISCPLRHPCDLSELTRRSR

SEQ ID NO: 19 - protein, 298 aa, Mycobacterium avium-paratuberculosis
MTYSPGSPGYPPAQSGGTYAGATPSFAKDDDGKSKLPLYLNIAVVALGFAAYLLNFGPTFTIGADLGPGIGGRAGDAGTAVVVALLAALLAGLGLLPKAKSYVGVVAVVAVLAALLAITETINLPAGFAIGWAMWPLVACVVLQAIAAVVVVLLDAGVITAPAPRPKYDPYAQYGQYGQYGQYGQQPYYGQPGGQPGGQPGGQQHSPQGYGSQYGGYGQGGAPTGGFGAQPSPQSGPQQSAQQQGPSTPPTGFPSFSPPPNVGGGSDSGSATANYSEQAGGQQSYGQEPSSPSGPTPA

SEQ ID NO: 20 - protein, 496 aa, Mycobacterium avium-paratuberculosis
VIPIPYLRARHRLAVDGVLLAMFVFGCFVFGVLSVRRTTEGVLLTAALFCVVVYWVKPEGMVGVTLFGAFAALPEGLHVGKVFGPLTIYAYHLAAFLAICYLIPAAKPRSSDFLLPGILAVTAVCSTVTGFLVGNSALVVTRESTTMLEMALGFVLALFVVYSGHVIWSIRVMIAILWFSAGMAIVSSLYSIRLAGRAESLEGTTGAGQAMRIILSTQTPATAVLSALVAAPIVGRVRPRLYLALGPPALSISLLSFSRNTLISMGVAAAVALLGSLSWAAVRRTIVAATVGATLVAVTVPGSLFLLQRSKTGAWLADQYVAFSQRVLGGVTSSALAVDDSALERLREINLLKETIASAPLFGHGLGYVYQPPTGDDEFHRYLYPAYSHNFYLWWLAKAGAVGMAAFVLFALTPVILALRCTSGPAKIAAAVAAGLLAISAVWPLPEMPMDALGLGMALGAAMGYAGLRRRERQLDDRCAAPGPTSNSPVGVGTSS

SEQ ID NO: 21 - protein, 858 aa, Mycobacterium avium-paratuberculosis
MMVDVTDVRDHHPKRGELRIYLGAAPGVGKTYSMLGEAHRRLERGTDLVAGVVETHGRAKTAELLEGIEIIPPRYIEYRGGRFPELDVPAVLARHPQVVLVDELAHTNTPGSKNPKRWQDVEELLDAGITVISTVNVQHLESLNDVVAQITGIEQKETVPDSVVRQASQVELIDITPEALRRRLSHGNVYAPDRIDAALSNYFRRGNLTALRELVLLWLADQVDTALAKYRAENKITDTWEARERVVVAVTGGPESETLVRRASRIASKSSAELMVVHVIRGDGLAGLSESRMAKIRELASSLDASLHTIVGDEVPAALLEFAREMNATQLVIGTSRRSRWARLFEEGIGPRIVELSGKIDVHLVTHEESKRGFRASSLAPRERRVASWLAALIVPSVICAVTVTWLDPYLDTGGESALFFVGVLLVGLLGGIAPAALSAVLSGLLLNYYLIAPRHSFTIAEPNSAITELVLLLIAVAVAVLVDFAAKRTREARRASQEAELLTLFAGSVLRGADLETLLERVRETYAQRSVSMLRESEDARAGGTKTQVVACVGRDPCVSVDAADTAIEVGGPDSSEFQMLLAGRKLSARDRRVLSAVARQAAGLIRQRELAEEASRTEAIVRADELRRSLLSAVSHDLRTPLAAAKVAVSSLRAEDVAFSPTDTAELLATIEESIDQLTALVGNLLDSSRLAAGAIHPDLRRVYLEEAVQRALVSIGKGATGFFRSAIDRVKVDVGDAMVMADAGLLERVLANLIDNALRYAPNCVVRVNAGQVGDRVLISVIDEGPGIPHGAEEQIFEAFQRLGDHDNTTGVGLGMSVARGFVEAMGGTITATDTPGGGLTVMVDMAAPQSEGAA

SEQ ID NO: 22 - protein, 274 aa, Mycobacterium avium-paratuberculosis
VTGFGARLAAAKAQRGPLCVGIDPHPELLRAWDLPTTADGLAAFCDICVEAFAGFAVVKPQVAFFEAYGAAGFAVLERTIAALRSAGVLVLADAKRGDIGTTMAAYAAAWAGDSPLAADAVTASPYLGFGSLRPLLEAAAAHDRGVFVLAATSNPEGATVQRAAFDGRTVAQLVVDQAAVVNRSTNPAGPGYVGVVVGATVLQPPDLSALGGPVLVPGLGVQGGRPEALAGLGGAEPGQLLPAVAREVLRAGPDVAELRAAADRMLDAVAYLDA

SEQ ID NO: 23 - protein, 416 aa, Mycobacterium avium-paratuberculosis
MDFGSLPPEINSGRIYSGPGSAPLLAAAAAWHGLAAEMHSAAASYGSAIAELRTLWHGPSSTAMAAAAAPFIAWLGGTAAQAEQTAAQATAAAAYDSVFAATVPPPVIAANRALLASLIATNVLGQNTPAIAATEAHYAEMWAQDAAAMYAYAGASAVATRLTPFGAPPQSADANAAADQSAAAASALQLSTASSVESALSQGVSQVPVAAQVNATAVTAAAQLPLSLTDITGILKTFNSVMGTISGPYTPLGVANLAKNWYQIALSIPSVGTGIQGIGPLLHPKALTGVLAPLLRSDLLTGSTALSSAGTVSASAGRAGLVGSLSVPANWASAVPAVRTVAAELPETMLDAAPAMAVNGQQGMFGPTALSSLAGRAVGGTATRAVAGSTVRVPGAVAVDDLATTSTVIVIPPNAK

SEQ ID NO: 24 - protein, 937 aa, Mycobacterium avium-paratuberculosis
VTDSVNSFGARNTLKVGDKSYQIYRLDAVPNTEKLPYSLKVLAENLLRNEDGSNITKDHIEAIANWDPKAEPSIEIQYTPARVVMQDFTGVPCIVDLATMREAIADLGGNPEKVNPLAPADLVIDHSVIADLFGTADTFERNVEIEYQRNGERYQFLRWGQGAFSDFKVVPPGTGIVHQVNIEYLARVVMERDGVAYPDTCVGTDSHTTMVNGLGVLGWGVGGIEAEAAMLGQPVSMLIPRVVGFKLTGEIQPGVTATDVVLTVTEMLRKHGVVGKFVEFYGEGVAEVPLANRATLGNMSPEFGSTAAIFPIDEETIDYLKFTGRNAEQVALVETYAKEQGLWHDPAHEPAFSEYLELDLSQVVPSIAGPKRPQDRIALSQAKSVFREQIPSYVGDGDGQQGYSKLDEVVDETFPASDPGAPSNGHADDLPAVQSAAAHANGRPSNPVTVRSDELGEFVLDHGAVVIAAVTSCTNTSNPEVMLGAALLARNAVEKGLASKPWVKTTMAPGSQVVHDYYDKAGLWPYLEKLGFYLVGYGCTTCIGNSGPLPEEISKAINDNDLSVTAVLSGNRNFEGRINPDVKMNYLASPPLVVAYALAGTMDFDFEKQPLGKDKDGNDVYLKDIWPSQKDVSDTIASAINSEMFTKNYADVFKGDERWRNLPTPSGNTFEWSPDSTYVRKPPYFEGMPAEPEPVADISGARVLALLGDSVTTDHISPAGSIKPGTPAAQYLDEHGVDRKDYNSFGSRRGNHEVMIRGTFANIRLRNLLLDDVAGGYTRDFTQDGGPQAFIYDAAQNYAAQNIPLVVLGGKEYGSGSSRDWAAKGTRLLGVRAVIAESFERIHRSNLIGMGVIPLQFPDGKSAKDLGLDGTEVFDITGIEELNKGKTPKTVHVKASKNGSDAVEFDAVVRIDTPGEADYYRNGGILQYVLRNMLKSG

SEQ ID NO: 25 - protein, 336 aa, Mycobacterium avium-paratuberculosis
MDFDAVLLLSFGGPEGPEQVRPFLENVTRGRGVPPERLDHVAEHYLHFGGVSPINGINRALIEQLRAAQDLPVYFGNRNWEPYVEDTVKVMRDNGIRRAAVFTTSAWSGYSSCTQYVEDIARARTAAGTGAPELVKLRPYFDHPLFVEMFAGAIADAAAKVPAGARLVFTAHSVPVAADERLGPRLYSRQVAYAARLVAAAAGYAEHDLVWQSRSGPPQVRWLEPDVADHLRALAESGTRAVIVCPIGFVADHIEVVWDLDEELRAQAESAGMLMARASTPNAQPRFARLAADLIDELRCGRTPARVTGPDPVPGCLASVNGAPCRPPHCAAQATG

SEQ ID NO: 26 - protein, 377 aa, Mycobacterium avium-paratuberculosis
MQGAVAGLVLLAVLVIFAIVVVAKSVALIPQAEAAVIERLGRYSRTVSGQLTLLVPFIDRIRARVDLRERVVSFPPQPVITEDNLTLNIDTVVYFQVTVPQAAVYEISNYIVGVEQLTTTTLRNVVGGMTLEQTLTSRDQINGQLRGVLDEATGRWGLRVARVELRSIDPPPSIQASMEKQMKADREKRAMILTAEGMRESAIKEAEGQKQAQILAAEGAKQAAILAAEADRQSRMLRAQGERAAAYLQAQGQAKAIEKTFAAIKAGRPTPEMLAYQYLQTLPEMARGDANKVWVVPSDFSAALQGFTKLLGTPGQDGVFRFQPSPVEDVPKHSADDDADVADWFSTETDPAIAQAVAKAEAIARQPADGPTGELTQ

SEQ ID NO: 27 - protein, 118 aa, Mycobacterium avium-paratuberculosis
MSALTSPKTYAALGVFHAVDAVACGVQVAPIRKTLDNLGVPDNIRPVLPVVKAAAAVGLLSVTRFPGLARLTTAMLTLYFVLAVGAHVRVRDKVVNGLPAALFVALFAAMTVRGPERS

SEQ ID NO: 28 - protein, 258 aa, Mycobacterium avium-paratuberculosis
LEGVTGSATSKIAETLRDLGCAIGAAARGVSRSRIAWTVAGITALVVLASLIPLPSPVQMRDWAQSVGPWFPLAFLLAHIVVTVVPVPRTAFTLAAGLLFGPLLGVAIAVAASTASAMIAMLLVRAAGWRLTRLVRHRSMDTVEERLRQRGWLAIVSLRLIPAVPFSALNYAAGASSVRVLPYGLATLAGLLPGTAAVVILGDALAGHPSSLLYLVSALTSALGLTGLVIEIRHFRRHHRRAHRHRDDEPSPEPATIG

SEQ ID NO: 29 - protein, 316 aa, Mycobacterium avium-paratuberculosis
VRSQRGGPRPVHEPGRTREVTAPRPDECRRGQERPGKMKRIYAFAIGLALLGAPAAPMVVPPVATADPGVRAMDYQQATDVVIARGLSQRGVPFSWAGGGINGPTRGTGTGANTVGFDASGLMQYAYAGAGIKLPRSSGAMYRVGQKILPQQARKGDLIFYGPEGTQSVAMYLGNNQMLEVGDVVQVSPVRTAGMAPYMVRVLGTTAPTQQVPQQAPLQQTPAQQAPLQQTPGQQAPLQQTPGQQLPTQQAPLQQVPGQQVPGQQLPTQQAPQQAPLQLAPTQQAPLQQLPTQQSPLQQLPVQQSPLQPAGAGLTR

SEQ ID NO: 30 - protein, 398 aa, Mycobacterium avium-paratuberculosis
VTGQRATPQPTLDDLPLRDDLRGKSPYGAPQLAVPVRLNTNENPHPPSRALVDDVVRSVARAAADLHRYPDRDAVQLRSDLARYLTAQTGVQLGVENLWAANGSNEILQQLLQAFGGPGRSAIGFVPSYSMHPIISDGTRTEWLQAARADDFSLDVDAAVAAVTERTPDVVFVASPNNPSGQSVSLSGLRRLLDAAPGIVIVDEAYGEFSSQPSAVQLVGEYPTKLVVTRTMSKAFAFAGGRLGYLIATPAVIEAMLLVRLPYHLSSVTQAAARAALRHADDTLGSVAALIAERERVSTALTGMGFRVIPSDANFVLFGEFTDAPASWQRYLDAGVLIRDVGIPGYLRATTGLAEENDAFLRASAQLAATELAPVNVGAIANAAEPRAAGRDRVLGAP

SEQ ID NO: 31 - protein, 266 aa, Mycobacterium avium-paratuberculosis
MDLDALVARASAILDDASKPFLAGHRADSAVRKKGNDFATDVDLAIERQVVAALVEATGIGVHGEEFGGSAVDSEWVWVLDPVDGTFNYAAGSPMAGILLALLHHGDPVAGLTWLPFLDQRYTAVTGGPLRKNEIPRPPLTSIDLADALVGAGSFSADARGRFPGRYRMAVLENLSRVSSRLRMHGSTGLDLAYVADGILGAAVSFGGHVWDHAAGVALVRAAGGVVTDLAGRPWTPASDSALAAGPGAHAEILDILRNIGRPEDY

SEQ ID NO: 32 - protein, 505 aa, Mycobacterium avium-paratuberculosis
VAEESRGQRGSGYGLGLSTRTQVTGYQFLARRTAMALTRWRVRMEVEPGRRQNLAVVASVSAALVICLGALLWSFISPAGQVGDSPIIADRDSGALYVRVGDRLYPALNLASARLITGRPDNPHLVKSNQIASLPRGPMVGIPGAPSNFHPTGPSTSSWLVCDTVSNSTGAGAPSGVTVTVIDAAPDLSNHRKVLTGSDAVVLNYGGDAWVIRDGRRSRIDATNRSVLLPLGLTPEQVSMAKPMSRALYDALPVGPELTVPQIQNAGGAASFPGAPGPIGTVLVTPQISGPQQYSLVLADGVQTLPPLVAQILQNAGPGNTKPVTVEPSALAKMPVVNKLDLSSYPDAPLNVMDIRENPATCWWWQKTSGENRARVQVVSGATIPVAQKDVNKVVSLVKADTTGREADQVFFGPDYANFVAVTGNDPGAKTTESLWWLTDAGARFGVDDTRDVREALGLKTKPSVAPWVALRLLPQGPTLSRADALVQHDTLPMDMSPAELAVPK

SEQ ID NO: 33 - protein, 407 aa, Mycobacterium avium-paratuberculosis
MLDYGAFPPEFNSARIYSGPGSGSLVAAASAWSSLAAELNAAALSYDKVVTALASEEWLGSASASMASAVAPYVGWMSTTAAQAEEAASQARAAAAAFEAALAASVPPPVIAANRMQVSQLQATNVLGQNTPLIAQFEAQYGEYWAQDAAAMYSYAGQSASASKVTPFQKAPQVTNPSGQVAQSAAVSTATANSTSTNTTKALQSLAQPASSSTTATKAATTAASTTSTDPLSEIWFLLTGQTTLPTSLGSAVNGYSPFASLFYNTEGLPYFSTGMANTFTQIAKSVGAIGGAAPAAAKALPGLGGLGGMLGGGGAAAAHPVAALGGAGSIGGKLSVPVAWSGAPAAPALGHAIPVSSISAAPEAAGGPGNLLGGMPLAGAGAGGHGAAGPKYGFRPTVMARPPFAG

SEQ ID NO: 34 - protein, 294 aa, Mycobacterium avium-paratuberculosis
LRDPVLVAIPFFLLLLTLEWTAARKLEHLTARPAPGAHQTRDSLTSISMGLVSVATTAGWKTLALFGYAAIYAYLAPWHLPATRWYTWAIAILGVDLLYYAYHRIAHRVRLIWATHQAHHSSEYYNFATALRQKWNNSGEILMWLPLPLLGIPPWMVFFSFSVNLIYQFWIHTERIDKLPRPFEFVFNTPSHHRVHHGMDKVYLDKNYGGILIVWDRLFGTFQAELFRPHYGLTKHVDTFNVWTLQTRESVAIARDWRSASRLRDRLGYVFGPPGWAPRSAGRTAAGAPVVTSL

SEQ ID NO: 35 - protein, 683 aa, Mycobacterium avium-paratuberculosis
MGRHSAPDPDDFLDEPSPDHPVDERDDAYAFDAQGAPDEGYYPDERRYPDADFVADDDYAPEEFAPGEDLVDEDPDDYPEFPSRRPATSGPQESPASAPSLRARRLDWRGGHRSEGGRRGVSIGVIVALVAVVVVVGSVILWRFFGDALSKRSHTAAGRCVGGQEQVPVVADPSIADAIGQFAESFNKSAGPIGDHCMVVSVKPAGSDAVLNGFIGKWPAELGGQPALWIPGSSVSAARLAGATAQKTITESHSLASSPVVLAVRPELLPALSGQNWAALPGLQTNPNALAGLNLPAWGSLRLALPMTGNGDAAFLAGEAVAAASVPPGAPVTQGTGAVRTLLSAQPKLADNSLTEAMNTLLKPGDSASAPVHAVVTTEQQLFQRGQSLPDAKGALASWLPPGAAAVADYPTVLLSGSWLTREQASAASEFSRFMHKSDQLAKLAKAGFRVNGGKPPSSPVTTFPALPSTLSVGDDAMRATLAEAMASPSTGQATTIMLDQSMPGQEGGKSRLANVIGALQDKIKALPASAVVGLWTFDGHEGRSEVTSGPLADPVNGQPRSAALSAALDKQYSSSGGAVSFTTLRMIYQDMQSNYHAGQTNSILVITAGPHTDQTLDGPGLQDFIRKSADPAKPIAVNVIDFGADPDRTTWEAVAQLSGGGYQNLATSASPDLATAVNAFLS

SEQ ID NO: 36 - protein, 388 aa, Mycobacterium avium-paratuberculosis
LSAATTADIDHYRTVLAGAFDDQVLEWTREAEARQRFPRELIEHLGARGVFSEKWCGGMLPDVGKLVELARALGRLSSAGIGVGVSLHDSAIAVLRRFGKSDYLRDICERAIAGQAVLCIGASEESGGSDLQIVRTEMSSRDGGFDIRGVKKFVSLSPIADHIMVVARSIDHDSASKHGNVALIAVPTSQASVQRPYAKVGAGPLDTAAVHIDTWVPADALVARAGTGLAAISWGLAHERMSIAGQIAASCQRAIGITLARMMTRRQFGRTLFEHQALRLRMADLQARVDLLQHGLNGIAAQGRLDLRAAAGVKVTAARLGEEVMSECMHIFGGAGYLVEETPLGRWWRDMKLARVGGGTDEVLWELVAAGMAADHGGYRSVVGASSA

SEQ ID NO: 37 - protein, 489 aa, Mycobacterium avium-paratuberculosis
MSSSVTPSRPTTGTAQIGVTGLAVMGSNIARNFARHGYTVALHNRSIAKTDALLKEHGDEGKFVRCETIAEFLDALEKPRRVLIMVKAGDPTDAVINELADAMEPGDIIIDGGNALYTDTIRREQAMRERGLHFVGAGISGGEEGALNGPSIMPGGPAESYRSLGPLLEEISAHVDGVPCCTHIGPDGAGHFVKMVHNGIEYSDMQLIGEAYQLLRDALGKTAEQIADVFDEWNSGDLDSFLVEITAQVLRQTDAKTGKPLVDLILDEAEQKGTGRWTVKSALDLGVPVTGIAEAVFARALSGSVAQRRATTGLASGRFGEKPSDAAQFTEDIRQALYASKIIAYAQGFNQIQAGSAEYGWDITPGDLATIWRGGCIIRAKFLNRIKDAFDENPDLPTLIVAPYFRSAIEAAIDGWRRVVVTATRLGIPIPGFSSALSYYDALRTERLPAALTQGLRDFFGAHTYGRIDEDPDKRFHTLWSADRREVPA

SEQ ID NO: 38 - protein, 461 aa, Mycobacterium avium-paratuberculosis
MSPHSGSTAGPERRHQVVIIGSGFGGLNAAKKLKHANVDIKLIARTTHHLFQPLLYQVATGIVSEGDIAPPTRVVLRRQRNVQVLLGDVTHIDLAGKFVVSDLLGHTYETPYDTLIVAAGAGQSYFGNDHFAEFAPGMKSIDDALEVRGRILSAFEQAERSRDPERRAKLLTFTVIGAGPTGVEMAGQIAELATYTLKGSFRHIDPTKARVILLDAAPAVLPPFGDKLGKRAADRLEKMGVEIQLGAMVTDVDRNGITVKDSDGTVRRIESACKVWSAGVSASPLGRDLAEQSTVELDRAGRVKVLPDLSIPGHPNVFVIGDLAAVEGVPGVAQGAIQGAKYVANTIKAELGGADPAEREPFQYFDKGSMATVSRFSAVAKIGPLEFSGLFAWFAWLVLHLVYLVGFKTKVSTLLSWTVTFLSTRRGQLTITEQQAFARTRLEQLAVLAAETKRPAARRAS

SEQ ID NO: 39 - protein, 368 aa, Mycobacterium avium-paratuberculosis
MDQVEATSTRRKGLWTTLAITTVSGASAVAIALPATSHADPEVPTPVPPSTATAPPAAPAPNGQPAPNAQPAPGAPAPNGQPAPAAPAPNDPNAAPPPVGAPPNGAPPPPVDPNAPPPPPADPNAGRIPNAVGGFSYVLPAGWVESDASHLDYGSALLSKVTGPPPMPDQPPPVANDTRIVMGRLDQKLYASAEANNAKAAVRLGSDMGEFFMPYPGTRINQDSTPLNGANGSTGSASYYEVKFSDASKPNGQIWTGVIGSANGGNAQRWFVVWLGTSNDPVDKVAAKALAESIQAWTPPAAPPAAPGGPGAPAPGAPGTPAAPGAPAAPAPAAPGAPAAPGAPAPGQAPAVEVSPTPTPTPQQTLSA

SEQ ID NO: 40 - protein, 233 aa, Mycobacterium avium-paratuberculosis
MEKVIAVLMRADSEEDWCARQRGVVADALLELGLPGLAVNVRDDAVRRSLMTLTTLDPPVAAVVSMWTQQSYGEQVAAALRLLAAECEQLAAYLVTESVPLPAPQTEPASRTPGLANIALLRRPAGMDQETWLTRWQRDHTPVAIETQSTFGYTQNWVVRTLTPGAPEIAGIVEELFPAEAITDLQAFFGAADEQDLQHRLGRMVASTTAFGANENIDTVPTSRYVVKTPFAQ

SEQ ID NO: 41 - protein, 247 aa, Mycobacterium avium-paratuberculosis
MTQIAFLAYPGFTALDMIGPYEVLRNLPGAEVRFVWHETGPITADSGVLVIGATHSLAETPSPDVILVPGGPGTAVHARDDALLDWLRAAHRTATWTTSVCTGSLILAAAGLLDGRRATSHWLTIPALKAFGVTAVPDERIVHEDGIVTSAGVSAGLDLALWLAAQIGGDGRAKAIQLALEYDPQPPFDSGHLSKASASTKAAATALLSRDSLSPTYLKATALLAWDQALDRVRSRRRRRQPDLSPA

SEQ ID NO: 42 - protein, 417 aa, Mycobacterium avium-paratuberculosis
MVRRIAGATCRSRESAWPAAVLVATTMLSVTACGHSGDNANHAAQSKPGGGNAVKITLTNSAGKDGCALDTTNVPAGPVTFTVANTNAPGISEVELLRDQRIVGEKENLAPGLDPVSFTLTLDGGSYQLYCPGASTEYQTLTVTGKAPATPTGTIATVLSQGTKDYAAYIVNQIGQLNDGAKALDAAVQAGNLDAAKAAYAKARLYWERSESTVEGFVLPGFAVGDNAGNLDYLIDMRESTPVDGKVGWKGFHAIERDLWQAGAITPGTKALSTELVGNVGKLHGIVATLQYKPEDLANGASDLIEEIQNTKITGEEEAFSHIDLVDFSGNVEGAQQAYASLRPGLEKIDNNLVHQIDQQFQNVLATLDGYRDPGALGGYRTYTPALKASDAPKLTAVIQPLHQSLSTVAQKVVSAG

SEQ ID NO: 43 - protein, 412 aa, Mycobacterium avium-paratuberculosis
LETWVMSISFETSESRADAELPVLPMPRAAHCPLAPPPEFVDWRQQPGLRRALFQGNPVWVVSRYHDIRAALVDPRLSAKTIPDSIMPTDADNKVPVMFARTDDPEHHRLRRMLTGNFTFRRCESMRPQIQDTVDHYLDRMLDGGAPADLVREFALPVPSLVIALLLGVPPEDLELFQFNTSKGLDQKSSDEEKGKAFGAMYAYIEELVQRKAREPGDDLISRLITEYVATGQLDHATTAMNSVIMMQAGHETTANMISLGTVALLGNPEIYARLGQTDDSAVVANIVEELMRYLSIVHSQVDRVATEDLTIAGQLIRAGEFVVMNLPAGNWDTEFVDNPESFDADRNTRGHLGFGYGVHQCIGANLARVEMQVAFATLARRLPGLRLAVPPEQLKFKDANIYGMKELPVSW

SEQ ID NO: 44 - protein, 383 aa, Mycobacterium avium-paratuberculosis
VLVVSTDQAHSLGDVLGVPVPPSQAELVRVLADLETGRAEAGGGFLDALALDTLALLEARWRDVVATLDRRFPDSELSTIAPEELSALPGVQEVLGLHAVGELARSGRWDRVVVDCASTADALRMLTLPATFGLYVERAWPRHRRLSLTAEDARSAAVVELLERVSASVEALSALLTDGDLVGAHLVLTPERVVAAEAARTLGSLALMGVRVEELIVNQVLLQDDSYEYRNLPEHPAFYWYTERIAEQQSVLEELDAAIGEVALVLTPHLSGEPIGPKALGALLDAARRRGGAAPPGPLRPTVDLESGTGLGSIYRMRLALPQLDPSALTLGRVDDDLIISAGGLRRRVRLASVLRRCTVLDAHLRGSELTVRFRPDPEVWPK

SEQ ID NO: 45 - protein, 250 aa, Mycobacterium avium-paratuberculosis
MAKSRSAADNKAARAQAQAARKAAARERRAQLWQAFNIQRQEDKRLLPYMIGAFLLVVGVSVGVGVWAGGLTMITLIPFGVVLGALVAFIVFGRRAQKSVYRKAEGQTGAAAWALDNLRGKWRVTPGVAATGHFDAVHRVIGRPGVILVGEGSPTRVRPLLAQEKKRTARLIGDVPIYDIIVGNGEDEVPLAKLERHLTRLPANITVKQMDTLESRLAALGSRAGAAVMPKGPLPNAGKMRGVQRTVRRK

SEQ ID NO: 46 - protein, 520 aa, Mycobacterium avium-paratuberculosis
VGMGLSRRGKSARTLLIWMSIAAVALLLAGCVRVVVGRAVMSGPKLGQAVEWTPCRAANPKVKLPAGALCGKLAVPVDYDHLDGDVATLAMIRFPATGDKIGSLVINPGGPGESGIEAALGVVQSLPKRVRERFDLVGFDPRGVGASRPAVWCNSDADNDRLRTEPNVDYSPAGVAHIEDETKQFVGRCVDKMGKKFLANVGTVNVARDLDAIRAALGDDKLTYLGYSYGTRIGSAYAEAYPHNVRAMILDGAVDPNADQIEADLRQAKGFQDAFNNFAAECAKQPNCPLGTDPAKAVDVYHSLVDPMVDPDNPMVGRPIPTNDPRGLSYSDAIVGTIMALYSPNLWHHLTDGLSELVDHHGDTLLALADMYMRRDAHGHYTNATDARVAINCVDQPPITDRAKVIDEDRRSREIAPFMSYGQFTGNAPLGTCAFWPVPPTSKPHTISAPGLAPTVVVSTTHDPATPYKAGVDLANELRSSLLTYDGTQHTVVFQGDGCIDNYVTAYLVGGTIPPSGAKC

SEQ ID NO: 47 - protein, 275 aa, Mycobacterium avium-paratuberculosis
MPRWLRGLSFLLRPGWVVLALVVVAFAYLCFTVLAPWQLGKHSRTSQQNHQIEHSLTTPPVPLKTLLPQQNSAAPAEQWRQVSATGHYLADVQVLARLRVIDSKPAFEVLAPFVVDGGPTVLVDRGYVRPLEGSRVPPIPRPPADTVTITARLRNSEPAAGKDPFVGDGVRQVYSIDTEQIAVLTKVPLAGSYLQLVDGQPGGLGVVGVPQLDAGPFLSYGIQWIAFGILAPIGVGYFAYSELRARRAERQPAAPAPEAPQSVQDKLADRYGRRR

SEQ ID NO: 48 - protein, 483 aa, Mycobacterium avium-paratuberculosis
LPATPIGLRAQLLRRRPVVGAHVAPGTADHLRRGIGTFQLTMFGVGSTIGTGIFFVMSQAVPEAGPAVIVSFLLAGVAAGLAAVCYAELASAVPVSGSSYSYAYTTLGEVVAMGVAACLLLEYGVATAAVSVNWSGYLNKLLSNVVGFQLPHALSAAPWDAQPGYVNLPAVMLIGMCALLLIRGASESAKVNAIMVMIKLGVLVVFGILAFTAFDVHHLDDFAPFGVAGVGTAAGTIFFSYIGLDAVSTAGDEVTNPQKTMPRALIAALSTVTGVYVFVALAALGTQPWQDFGGQQEAGLATILDHVTHGSWASTILAAGAVISIFSVTLVTMYGITRILFAMGRDGLLPPRFARVNPRTMTPVNNTVIVAVAASTLAAFIPLQNLADMVSIGTLTAFVVVSVGVIVLRVREPDLPRGFRVPGYPVTPVLSIMACGYILASLHWYTWIAFSGWVLLALIFYFVWGRHHSALNDAAVDPSGQER

SEQ ID NO: 49 - protein, 586 aa, Mycobacterium avium-paratuberculosis
MITSKLRAQRPSFRTDEANSTHRLPLRTAARTTGVVAYQLGLSVDGHETLSGISFTAKPGTMTAVIGPSPARNAALLALLAGTRTPSSGRVTVDGHDVHAEPAAMRARIGVVSREERLHRRLTVEQALRYAAELRLPPETSAEQRDRVVGQVLDELDLTTHRDTRIRKLAPEVRRCTALAIELVTRPSLLVVDEPTAGLNAAQQRHVMAVLRRQANLGCVVVAAISSRTSLTDVNMCDQVLVLTAAGKVAYLGTPLQAESAMGSADWSAVLARVGADPDGAHRAFRARPQSAAPTIPPEVAAPWAPPAALPVPRQVRCVARREIRLLLANRLYFAFLALLPFVLAGLTLLIPGDSGLARPAPSSANAHEAIEILALLNVAAVIIGTALTVPAMVGEHRVYRREQQVGLSAPAYLAAKIAVYALAAAVWAAVMLAVVIAVKGAPVYGAVVLHDATFELYVAVAVTAMVSAVIGLALSALGKSLGEVLPLLVPVILAAVLFNGSLVQLVSMWGLQQISWLIPARWGFAASASTVNLRRIDPLAANAETWTHYSGWWVFDMVMLVLFGVAAAGVTLYRLRSPGKIRSAT

SEQ ID NO: 50 - protein, 255 aa, Mycobacterium avium-paratuberculosis
VNATAIAKPMTALGQFFLLSAEALAAAVRGPWAWREILEQIWFVARVSIFPTIMLSIPYTVLIVFVLNILLVEIGAGDLSGAGAGLASVTQVGPVVTAMVVSGAGSTAMCADLGARTIREEIDAMKVIGVNPVQALVVPRIIAATFVAVMLYAVVAVIGLTGSYIFVVFVQHVTPGAFVAGMTLVTGLPQVVISLIKATLFGLSAGLIACYKGLSVGGGPTGVGNAVNETVVFSFMALFFINILTTALGVKVTAK

SEQ ID NO: 51 - protein, 310 aa, Mycobacterium avium-paratuberculosis
MSIAENVTQLIGNTPLVRLNRVTEGAVADVVAKLEFFNPGNSVKDRIGVAMIDAAEQAGLIKPDTIILEPTSGNTGIALALVAAARGYRCVLTMPETMSVERRMLLRALGAEIVLTPGADGMPGAIAKAEELAKSDDRYFVPQQFENPANPAIHRSTTAEEVWRDTDGKVDIFVAGVGTGGTITGVAQVIKERKPSAQFIAVEPAASPVLSGGQKGPHPIQGLGAGFVPPVLAMDLVDEVIAVGNEESIALARRLAAEEGLLVGISSGAALVAALQVARRPENAGKLVVVVLPDFGERYLSTPLFADLAD

SEQ ID NO: 52 - protein, 193 aa, Mycobacterium avium-paratuberculosis
MDQDDLPRTARVSIVAPSPEGELAEVALLFTNIVRRDTAAFREELQNLVNSLAETSETKPVITESQTPYPGGGLAQYGIAFAVGLPTALAYNVIYDALKKLSHRFSWTAGSPPQERFLMENANPLALGAIEQGFGVARDDLRPVVVDVQGLRAHVVYHAKDGSMFTVEMENTGQFAITSVRKNWPNAGWGDES

SEQ ID NO: 53 - protein, 360 aa, Mycobacterium avium-paratuberculosis
LRVELLVKIEYGSTVTWYLGVVVTIVAEQRTYVAGRWVTGDEVVSVENPADESHVADITVTPLPEVQRAIAEARRSFDDGVWADMPPVERAQILHAFIDHIESERATLVPTLVAEAGQSARFAEMTQLGAGAAIARQTIDLYLSMSHEEASPVPVDDLVRGRVALSVRRHEPVGVVTAITPYNAALIMGFQKLIPALMAGNSVILRPSPLTPISSLIFGAAADAAGLPPGVLSVVVESGIAGAELLTSDPSVDMVSFTGSTLAGRKILAQAAPTVKRVSLELGGKSAQIYLPDAVHRAVGGAFVAVASTAGQACVAATRLLVPQDKKAEVLDAVSAMYQQIKVGPPSDETAMMGPVISAA

SEQ ID NO: 54 - protein, 448 aa, Mycobacterium avium-paratuberculosis
MGLLGCRRIWHGPTRRLVLRRRWRSRSSGSVKHPCGPGRRRPGVADSEFVVASPAGDTVDQIDTVPIDSGASVPPSGNPVSLIAAACCEHRTNVDPEVQTQVAIEEWMGASPNYTRRLRHALGVTGDTVEDIFKVLQFDVGAPPQFLDFRYSLIDPNHGEFRNDYCGALIDVEPMGDVWVRAMCHTIQDFTFDATAIATNPKARFRPIHRPPRKPADRTPHCHWSVTIEDSREDLPIPAEAVEVSRCELTALQFDPIDLSDDGLGDYTGPLFSDIRFDQWSRSALVRLAEEVAIQHHLLALAFERSVRRHGGEAKALGLLRRQFTGTAYVGSARIKAASGLASAQMTLLRSSICIPPCARSPTPEHPWSASGQERATRCGCASQATPPPSATAVGWRRCRRTMSVPSRSWPPVSIRTGRICTRPTRPATWSSTSGGRTPKRSAGRKSK

SEQ ID NO: 55 - protein, 882 aa, Mycobacterium avium-paratuberculosis
VTASRSPATDLPKSWDPPAAEYAIYRQWVDAGYFTANPASDKPGYSIVLPPPNVTGSLHMGHALEHTMMDALTRRKRMQGYEVLWQPGMDHAGIATQSVVEKQLAVDGKTKEDFGRELFIEKVWDWKRESGGAIGGQMRRLGDGVDWSRDRFTMDEGLSRAVRTIFKRLYDAGLIYRAERLVNWSPVLQTALSDIEVNYEEVEGELVSFRYGSLDDSGPHIVVATTRVETMLGDTAIAVHPDDERYRHLVGSSLPHPFVDRQLLIVADEHVDPEFGTGAVKVTPAHDPNDFEIGLRHQLPMISIMDTRGRIADTGTQFDGMDRFAARVAVREALAAQGRIVEEKRPYLHSVGHSERSGEPIEPRLSLQWWVRVESLAKAAGDAVRNGDTVIHPTSMEPRWFAWVDDMHDWCVSRQLWWGHRIPIWYGPNGEQRCVGPDETPPEGWEQDPDVLDTWFSSALWPFSTLGWPEKTPELEKFYPTSVLVTGYDILFFWVARMMMFGTFVGDDDAITLDGRRGPQVPFTDVFLHGLIRDESGRKMSKSKGNVIDPLDWVDMFGADALRFTLARGASPGGDLAIGEDHVRASRNFCTKLFNATRYALLNGAQLAELPPLDELTDADRWILGRLEEVRAEVDSAFDNYEFSRACESLYHFAWDEFCDWYVELAKTQLAEGITHTTAVLATTLDTLLRLLHPVIPFITEALWQALTGNESLVIADWPRSSGIDLDQVATQRITDMQKLVTEVRRFRSDQGLADRQKVPARLAGVTESDLDTQVSAVTSLAWLTDAGPDFRPSASVEVRLRGGTVVVELDTSGSIDVAAERRRLEKDLAAAHKELASTTAKLANEDFLAKAPPHVVDKIRDRQRLAQEESERINARLAVLQ

SEQ ID NO: 56 - protein, 158 aa, Mycobacterium avium-paratuberculosis
VAPVARGEVATREPAELPNGWVITTSGRISGVTEPGELSVHYPFPIKDLVAIDDALKFGSRASKTRFAIYLGDLGTDTAARAREILADVPTPDNAVLLAVSPDQKVIEVVYGSAVRGRGAESAAPLGVAAASSAFQRGDLVDGLVSAIRVLSAGISPA

SEQ ID NO: 57 - protein, 275 aa, Mycobacterium avium-paratuberculosis
MSSALAPVGIFDSGVGGLTVARAIIDQLPDEHIIYVGDTGHGPYGPLSIPEVRAHALAIGDDLVGRGVKALVIACNTASAACLRDARERYEVPVVEVILPAVRRAVATTRNGRIGVIGTQATINSHAYQDAFAAARDTEITAVACPRFVDFVERGVTSGRQVLGLAEGYLEPLQRAQVDTLVLGCTHYPLLSGLIQLAMGDNVTLVSSAEETAKEVLRVLAERDLLHPHPDDPRAAGPSRVFEATGDPEAFTRLAARFLGPAVSGVRPVHHVRID

SEQ ID NO: 58 - protein, 417 aa, Mycobacterium avium-paratuberculosis
VAERFVVTGGNRLSGEVAVGGAKNSVLKLMAATLLAEGTSTITNCPDILDVPLMAEVLRGLGATVELDGDVARITSPDEPKYDADFAAVRQFRASVCVLGPLVGRCKRARVALPGGDAIGSRPLDMHQAGLRQLGATCNIEHGCVVAQADTLRGAEIQLEFPSVGATENILMAAVVAEGVTTIHNAAREPDVVDLCTMLNQMGAQVEGAGSPTMTITGVPRLYPTEHRVIGDRIVAATWGIAAAMTRGDISVTGVDPAHLQVVLHKLHDAGATVTQTDDSFRVTQYERPKAVNVATLPFPGFPTDLQPMAIALASIADGTSMITENVFEARFRFVEEMIRLGADARTDGHHAVVRGLPQLSSAPVWCSDIRAGAGLVLAGLVADGDTEVHDVFHIDRGYPLFVENLAILGAEIERVE

SEQ ID NO: 59 - protein, 199 aa, Mycobacterium avium-paratuberculosis
LVMAVHLTRIYTRTGDDGTTGLSDFSRVSKNDPRLVAYADCDEANAAIGVAVAVGRPGPELAGVLRQIQNDLFDAGADLSTPVVEDPEYPPLRVTQPYIDRLEKWCDTYNESLPKLNSFVLPGGSPLSALLHVARTVVRRAERSAWAAVDAAPEGVSALPAKYLNRLSDLLFILSRVANPDGDVLWKPGGQQGGEPAPG

SEQ ID NO: 60 - protein, 189 aa, Mycobacterium avium-paratuberculosis
MMAMMRPGPRRSTARAAATVLFLALLVLTGCSRSIAGNAVKAGGNVPRNNNSQQQYPNLLKECEVLTSDILAKTVGADPLDIQSTFVGAICRWQAANPAGLIDITRFWFEQGSLSNERKVAEFLKYKIETRNIAGIDSIVMRPDDPNGACGVASDAAGVVGWWVNPQAPGIDACGQAIKLMELTLATNS

SEQ ID NO: 61 - protein, 110 aa, Mycobacterium avium-paratuberculosis
MRLSLSKLGVAVGSAAVALTAAAGVASADPMDAIINTTCNYGQVIAALNASDPAAAQQLNSSPMAQSYIQRFLASPPAKRQQMAQQIQGMPAAQQYINDINQVAVTCNNF

SEQ ID NO: 62 - protein, 351 aa, Mycobacterium avium-paratuberculosis
VTAVDDSKDGFSMTAPPGGIYGPGSYGSNPYGQEPNWGGQPPGGQPPGGQPQGGPYPQPGQYPAGGPYPYPPPGGGYPYPGGPYPGGPYPGAPYPGPGQPFGPGGPYSPGPPPGGPGSKLPWLIVAGLVVLAVIALVATLVVMKGGHGSKPSGATPSSTSTSVSQPKNSAQNATDCTPNVSGGDMPRSDSIAAGKLSFPANAAPSGWTVFSDDQGPNLIGALGVAQDVPGANQWMMTAEVGVTNFVPSMDLTAQATKLMQCLANGPGYANAMPTLGPIKTSPITVDGTKAVRADADVTIADPTRNVKGDSVTIIAVDTKPVSVFIGSTPIGDSASAGLIGKIIAALKVAKS

SEQ ID NO: 63 - protein, 786 aa, Mycobacterium avium-paratuberculosis
LEQSNASPATRRIVSGSFPRAIAARSPETQYGRRRKGSHARRLEGLVKVHRGRMRKLVGSALVSLTTTALAAVLLAPAATASPIGDAEAAIMAAWEKAGGDTSPLGARKGDVYPVGDGFALDFDGGKMFFTPATGAKFAYGPILDKYESLGGPAGSDLGFPAINEVPGLAGPDSRVVTFSASDKPVIFWTPEHGAYVVRGAINSAWDKLGSSGGVLGVPVGDETYNGEVSTQKFSGGQVSWNRQTKQFSTEPPGLADQLKGLQVAIDPTAAINTAWRAAGGPGGPLGAKQGGPTPVGGDGIVQNFAGGKVFFTPATGANALESDILAKYESLGGPAGSDLGFPTTNETDGGIGPSSRIATFSAPDKPVIFWTADHGAFVVRGAMRAAWDKLRAPAGKLGAPVGDQAVDGDVISQQFTGGKISWNRAKNAFSTDPSNLAPLLSGLQISGQNQPSSSAMPAHPKKFSWHWWWLMAAVPVAVLLVLLIWVLFVWRRRRPGPEATGYGVDHGYDAAEGQWGHDDADVATEHFGAPPSGEPPAGSGAAARVSWQRQAPADGGYGFEEEDPDAVDTDSIPVVSDEMLAEADYPAAEADYTDYTDAVPEVAEPETADDAAYADADYAEVDYPDVGYREDEYPDLAVPHTPPDADAVTGGIPAAEADDEYAELAAPQAQPEERPEPQPGPEEVAEAAGGAVAAGVAGTRPRSGRHAAADEEDASENGLAGPDGRPTIHLPLEDPYQAPEGYPIKASARYGLYYTPGSDLYRDTLPELWLSSEEVAQANGFTKAD

SEQ ID NO: 64 - protein, 280 aa, Mycobacterium avium-paratuberculosis
VIAVTIEDPAIMPEAFFTVDGDSYVPGTMTRGPWGAAMGGQIVGGLLGWGIEQSGVDPDLQPARFTVDLLRPALLAPVQIRTSVQREGRRIKLVDAGLVQNGVVVARASALFLRRGDHPDGQVWSPPVQMPPLPTSSEGFPADMPFLIWGYGATRAGSPGIAAGEWEQAHSQKFAWARLFRPMVHGHPLTPFTRLAFVGDITSSLTHWGTGGLRYINADYTVSASRLPDGEFLGLAAQSHYGTAGVAAGAATLFDRHGPLGTSWALALAQPADAFQPAYT

SEQ ID NO: 65 - protein, 757 aa, Mycobacterium avium-paratuberculosis
MSVAEIEEGVFEATATIDNGSFGTRTIRFETGRLAQQAAGAVVAYLDDENMLLSATTASKSPKEHFDFFPLTVDVEERMYAAGRIPGSFFRREGRPSTDAILTCRLIDRPLRPSFVDGLRNEIQVVVTILSLDPNDLYDVLAINAASASTQLGGLPFSGPIGGVRVALIDGTWVAFPTVEQLERAVFDMVVAGRKVDGADGPDVAIMMVEAEATSNVIELIDGGAQAPTETVVAQGLEAAKPFIEVLCTAQQELADKAARPTSDYPTFPDYGDDVYYSVASVATDELSKALTIGGKAERDARTDELKAEVLARLAETYEGREKEVSAAFRSLTKKLVRQRILTDHFRIDGRGITDIRALSAEVAVVPRAHGSALFQRGETQILGVTTLDMVKMAQQIDSLGPETTKRYMHHYNFPPFSTGETGRVGSPKRREIGHGALAERALVPVLPSLEDFPYAIRQVSEALGSNGSTSMGSVCASTLALLNAGVPLKAPVAGIAMGLVSDDIEVEAGDGTKSLERRFVTLTDILGAEDAFGDMDFKVAGTKDFVTALQLDTKLDGIPSQVLAGALSQAKDARLTILEVMAEAIDEPDEMSPYAPRVTTIRVPVDKIGEVIGPKGKIINAITEETGAQISIEDDGTVFVGATDGPSAQAAIDRINAIANPQLPTVGERFLGTVVKTTDFGAFVSLLPGRDGLVHISKLGKGKRIAKVEDVVNVGDKLRVEIADIDKRGKISLVLVEEDNSAPADTPAAAPADATS

SEQ ID NO: 66 - protein, 459 aa, Mycobacterium avium-paratuberculosis
METYDLAIIGTGSGNSLLDARFAGKRTAICEHGTFGGTCLNVGCIPTKMFVYAADVATTIREAARYGVDTHLDGVRWPDIVSRVFGRIDPIALSGEEYRRSSVNIDLYRSHTRFGPVQFDGRYLLRTDAGEQFTAEQVVIAAGSRPVIPPAILESGVTYHTSDTIMRIPALPEHLVIVGSGFVAAEFAHIFSALGVHVTVVIRSGRMLRQYDDMICERFTRLAAAKWELRTQRNVVGGSNRGSGVTLRLDDGSTLDADVLLVATGRISNADLLDAGQAGVDVENGRVVVDEYQRTSARGVFALGDVSSPYQLKHVANHEARVVRHNLLCDWDDTESMAVTDHRYVPSAVFTDPQLATVGLTENQAIARGFDISVAIQNYGDVAYGWAMEDTTGVVKLIAERTSGRLLGAHIMGPQASSIIQPLIQAMSFGLTAAQMARGQYWIHPALPEVVENALLGLY

SEQ ID NO: 67 - protein, 461 aa, Mycobacterium avium-paratuberculosis
LTGSDTAMLSLAALDRLVAAGAPETRVDTVIVAFPDMQGRLVGKRMDARLFVDEAAATGVECCGYLLAVDVDMNTVGGYAISGWDTGYGDLVMRPDLSTLRRIPWLPGTALVIADVVGADGSPVAVSPRAVLRRQLDRLAGRGLFADAATELEFMVFDEPYRQAWASGYRGLTPASDYNIDYAISASSRMEPLLRDIRRGMAGAGLRFESVKGECNRGQQEIGFRYDEALRTCDNHVIYKNGAKEIADQHGKSLTFMAKYDEREGNSCRVHLSLRDAQGGAAFADPSRPHGMSTMFCSFLAGLLATMADFTLFYAPNINSYKRFADESFAPTALAWGLDNRTCALRVVGHGAHTRVECRVPGGDVNPYLAVAAIVAGGLYGIEQGLALPEPCAGNAYRARGVGRLPGTLAEAAALFEHSALARQVFGDDVVAHYLNNARVELAAFHAAATDWERMRGFERL

SEQ ID NO: 68 - protein, 172 aa, Mycobacterium avium-paratuberculosis
VRLQGMSRLSFVCRLLAATAFAVALLLGLGDVPRAAATDDRLQFTATTLSGAPFNGASLQGKPAVLWFWTPWCPYCNAEAPGVSRVAAANPGVTFVGVAAHSEVGAMANFVSKYNLNFTTLNDADGAIWARYGVPWQPAYVFYRADGSSTFVNNPTSAMPQDELAARVAALR

SEQ ID NO: 69 - protein, 152 aa, Mycobacterium avium-paratuberculosis
LGTRPALARVSSGSVANVTAGRRSSSPLFRRARAQEPRWKRSPSMSSQRTVRLSSSVRTVTPRSSATVRNRTISAESSTGSSSNGADGGQSITGISRPKVKNPTARCATGDTLITTGAAAGGGDGTGDDEPLATRPSFHPDQRGAHGHGFDC

SEQ ID NO: 70 - protein, 485 aa, Mycobacterium avium-paratuberculosis
MNAEPRTGPAKTLASALARDIEAEIVRRGWAVGESLGSEPALQQRFGVSRSVLREAVRLVEHHQVARMRRGPNGGLYICEPDAGPATRAVVIYLEYLGTTLADLLNARLVLEPLAASLAAERIDEAGIARLRAVLHAEQQWRPGLPMPRDEFHIALAEQSKNPVLQLFIKVLMRLTTRYALQSRTDSETEALEAVDHLHTHHSRIVAAVTAGDPARAKTLSERHVEAVTAWLQRHHAGDRNRGRTPRRPLNSEVPQGKLAEMLAATIGDDIAADGWRVGSVFGTETALLQRYRVSRAVFREAVRLLEYHSIAHMRRGPGGGLVIAEPAAQASIDTIALYLQYRDPSREDLRCVRDAIEIDNVAKVVKRLAEPQVAAFVASRRSGLPDDSRQTPDDVRRAIAEEFDFHVGLAQLAGNAPLDLFLRIIVELFRRHWSSTGQALPTWSDVRAVHHAHLRIADAVAAGDLSVASYRLRRHLDAAASWWL

SEQ ID NO: 71 - protein, 284 aa, Mycobacterium avium-paratuberculosis
VTVEPPPDHVLSAFGLAGVKPVYLGASWEGGWRCGEVVLSLVADNARAAWSARVRETLFVDGVRLARPVRSTDGRYVVSGWRADTFVAGTPEPRHDEVVSAAVRLHEATGKLERPRFLTQGPTAPWGDVDIFIAADRAAWEERPLASVPPGARVAPATADAQRSVELLNQLATLRKPTKSPNQLVHGDLYGTVLFVGSAAPGITDITPYWRPASWAAGVVVIDALSWGEADDGLIERWNALPEWPQMLLRALMFRLAVHALHPRSTAEAFPGLARTAALVRLVL

SEQ ID NO: 72 - protein, 546 aa, Mycobacterium avium-paratuberculosis
MTSVTDHTAEPAAEHSIDIHTTAGKLAELHKRREESLHPVGEEAVEKVHAKGKLTARERILALLDEDSFVELDALARHRSKNFGLENNRPLGDGVITGYGTIDGRDVCIFSQDATVFGGSLGEVYGEKIVKVQELAIKTGRPLIGINDGAGARIQEGVVSLGLYSRIFRNNILASGVIPQISLIMGAAAGGHVYSPALTDFVVMVDQTSQMFITGPDVIKTVTGEDVTMEELGGAHTHMAKSGTLHYVASGEQDAFDWVRDLLSYLPPNNATDPPRYAEPHPAGAIEDNLTDEDLELDTLIPDSPNQPYDMHEVITRILDDDEFLEIQGGYAQNIVVGFGRIDGRPVGIVANQPTQFAGCLDINASEKAARFVRTCDCFNIPIIMLVDVPGFLPGTGQEYNGIIRRGAKLLYAYGEATVPKITVITRKAYGGAYCVMGSKDMGCDVNIAWPSAQIAVMGASGAVGFVYRKQLAEAAKKGEDVDALRLQLQQEYEDTLVNPYVAAERGYVDAVIPPSHTRGYIATALRLLERKIAHLPPKKHGNIPL

SEQ ID NO: 73 - protein, 210 aa, Mycobacterium avium-paratuberculosis
LTRLVLASASAGRLKVLRQAGVDPLVVVSGVDEDAVIAALGPDASPSAVVCALATAKADRVAGALQAGVAADCVVVGCDSMLFIDGGLCGKPGSADAALRQWRRIGGRSGGLYTGHCLLRLRDGDITHREVESACTTVHFASPVEADLRAYVAGGEPLAVAGGFTLDGLGGWFVDGIDGDPSNVIGVSLPLLRTLLTRVGLSVSALWAAD

SEQ ID NO: 74 - protein, 271 aa, Mycobacterium avium-paratuberculosis
VAETPSNPGELARQAAAVIGERTGVAEHDVAIVLGSGWSPAVAALGTPTAVLPQAELPGFRPPTAVGHTGELVSMRIGEHRVLVLVGRIHAYEGHDLCHVVHPVRAACAAGVRAVVLTNAAGGLRPDLAVGEPVLISDHLNLTGRSPLVGPQFVDLTDAYSPRLRELARQADPTLAEGVYAGLPGPHYETPAEIRMLRTLGADLVGMSTVHETIAARAAGAEVLGVSLVTNLAAGISGEPLSHTEVLAAGAASATRMGALLALILCQLPRF

SEQ ID NO: 75 - protein, 531 aa, Mycobacterium avium-paratuberculosis
VTTVPAMTAPIWMASPPEVHSALLSSGPGPASMFAAAAAWSALGAEYASAAEELSGLLASAQAGAWQGPSAASYVAAHGPYLAWLTRASAHSAAAAAQHETAGTAYTAALAAMPTLPELAANHAVHGALVATNFFGINTIPIAVNEADYARMWVQAAGTMATYQAVSTAAVAAVPQPDPAPSILKSTAAHDHDDHEHGDDHDHDHGFDSPLNQFVAQILRLFGIDWDPVEGTLNGLPYEAYTSPADPLWWVVRALELFSDFQQFGALLQENPAAAFQFITELVLLDWPTHLAQLASWLPTQPQLLLVPALVAAAPFGALAGFAGVAGQPPLPAPVAEPATPSAAAPTGLPATAGATPIAASAAASGPAPAPTPAPTAATVSSPAPPAPPAPGAAPFAPPYAVPPPGAGFGSKARASVDTRAKSKSPQPDSNAVGAGAAVREAAHARRRQRSRRRGDEFMDMNVGVDPDWDEPATTASPRGAGNLGFAGTAPRETVAAAGLTQLAGDEFGGGAGMPLLPGSWAPPDPRDSGV

SEQ ID NO: 76 - protein, 361 aa, Mycobacterium avium-paratuberculosis
MSKSHHHRSVWWSWLVGVLTVVGLGLGLGSGVGLAPASAAPSGLALDRFADRPLAPIDPSAMVGQVGPQVVNIDTKFGYNNAVGAGTGIVIDPNGVVLTNNHVISGATEISAFDVGNGQTYAVDVVGYDRTQDIAVLQLRGAAGLPTATIGGEATVGEPIVALGNVGGQGGTPNAVAGKVVALNQSVSATDTLTGAQENLGGLIQADAPIKPGDSGGPMVNSAGQVIGVDTAATDSYKMSGGQGFAIPIGRAMAVANQIRSGAGSNTVHIGPTAFLGLGVTDNNGNGARVQRVVNTGPAAAAGIAPGDVITGVDTVPINGATSMTEVLVPHHPGDTIAVHFRSVDGGERTANITLAEGPPA

SEQ ID NO: 77 - protein, 352 aa, Mycobacterium avium-paratuberculosis
MSFIEKVRKLRGAAATMPRRLAIAAVGASLLSGVAVAAGGSPVAGAFSKPGLPVEYLEVPSPSMGRNIKVQFQGGGPHAVYLLDGLRAQDDYNGWDINTPAFEEFYQSGLSVIMPVGGQSSFYSNWYQPSSGNGQNYTYKWETFLTQEMPLWMQSNKQVSPAGNAAVGLSMSGGSALILAAYYPQQFPYAASLSGFLNPSEGWWPTLIGLAMNDSGGYNANSMWGPSTDPAWKRNDPMVQIPRLVANNTRIWVYCGNGTPSDLGGDNVPAKFLEGLTLRTNEQFQNNYAAAGGRNGVFNFPANGTHSWPYWNQQLMAMKPDMQQVLLSGNITAAPAQPAQPAQPAQPAQPAT

SEQ ID NO: 78 - protein, 233 aa, Mycobacterium avium-paratuberculosis
MDAVDPDSRHQLAVRMAELVRGMAAPRRLDQVLAEVTAAAVEVIPGADIAGVLLVRKGGEFETLADTDSLAARLDVLQHDFGEGPCAQAALQETIVRSDDLRREPRWPRYAPAAVQLGVLSSLSFKLYTADRTAGALNLFSHRPDAWDTEAETIGSVFAAHAAAAILAGSRAEQLYSAVSTRDRIGQAKGIIMERFGVDDVRAFDLLRRLSQESQVKLVEIAQQIIDTRGQGA

SEQ ID NO: 79 - protein, 105 aa, Mycobacterium avium-paratuberculosis
MYDELLANLAILVLSGFVGFAVISKVPNTLHTPLMSGTNAIHGIVVLGALVVFGSVEHPSLAMQIILFVAVVFGTLNVIGGFIVTDRMLGMFKSKKPAKADEAAK

SEQ ID NO: 80 - protein, 474 aa, Mycobacterium avium-paratuberculosis
MNYLVIGLYIVSFALFIYGLMGLTGPKTAVRGNLIAAVGMAIAVAATLIKIRHTDQWVLIIAGLVVGVVLGVPPARYTKMTAMPQLVAFFNGVGGGTVALIALSEFIETSGFSAFQHGESPTVHIVVVSLFAAIIGSISFWGSIIAFGKLQEIISGAPIGFGKAQQPINLLLLAGAVAAAVVIGLHAHPGSGGVSLWWMIGLLAAAGVLGLMVVLPIGGADMPVVISLLNAMTGLSAAAAGLALNNTAMIVAGMIVGASGSILTNLMAKAMNRSIPAIVAGGFGGGGVAPGGGDGGDKHVKSTSAADAAIQMAYANQVIVVPGYGLAVAQAQHAVKDMAALLEEKGVPVKYAIHPVAGRMPGHMNVLLAEAEVDYDAMKDMDDINDEFARTDVAIVIGANDVTNPAARNEASSPIYGMPILNVDKAKSVIVLKRSMNSGFAGIDNPLFYAEGTTMLFGDAKKSVTEVAEELKAL

SEQ ID NO: 81 - protein, 408 aa, Mycobacterium avium-paratuberculosis
MKFALAVYGSRGDVEPHAAIARELLRRGHEVCVAAPPDLRGFVESAGVTAIDYGPDTRDVLFGKKTNPIKLLSTSKEYFGRIWLEMGETLTSLANGADLLLTAVAQQGLAANVAEYCDIPLATLHCLPARVNGRLLPNVPSPLSRLAVSAFWCGYWCVTNKAEESQRRRLGLSKASGSSTRRIVGRKSLEIQAYEDFLFPGLAAEWAHWDGQRPFVGALTLGLPTDADAEVLSWIAAGSPPVYFGFGSLPVKSPADTVAMISAACTRLDERALICAGTNDLTHVPRSGHVKIVAAMNHAAIFPACRAVVHHGGAGTTAAGMRAGVPTLVLWMRNEQPLWGAAVKQMKVGSSQRFSKTTEESLATCLRSILRPHYMTRAREVAKRMTKSSDSAAVAADLLENAARGETT

SEQ ID NO: 82 - protein, 139 aa, Mycobacterium avium-paratuberculosis
VSSPAAPRRRRATVKQRTVLEVLRAQENFRSAQQLYQDIRQNQQLRIGLTSVYRILRALAADRIAETQRAEDGEILYRLRTEAGHRHYLLCRQCGRAVAFTPVDIEEHTRRLSRQHHYADVTHYVDLYGTCPLCQNTQP

SEQ ID NO: 83 - protein, 195 aa, Mycobacterium avium-paratuberculosis
LSGCSTPSRLSLFRSTLSSFGRPGVRGTRRAMTQTTQPLMRTQVRADIPDSERDPARARRGGKRVARLRAGAVCWLAIAVCCLAAAGLAATGARTGLGGGSPAPVVPEAGTLQVSGAGTTKSLPCHAGYLSVSGKDNTVTLTGHCTSVSVSGNGNRIAVDSSDAVSAAGAGNVVVYHWGSPKVVNAGSGNVVRQG

SEQ ID NO: 84 - protein, 214 aa, Mycobacterium avium-paratuberculosis
VTNPHFAWLPPEVNSALIYSGPGPGPLLAAAAAWDGLAEELASSAQSFSSVTSDLASGSWQGASSAAMMTVANQYVSWLSAAAAQAEEVSHQASAIATAFEVALAATVQPAVVAANRALVQALAATNWLGQNTPAIADIEAAYEQMWASDVAAMFGYHADASAAVAKLPPWNEVLQNLGFSNASTAVTRPAGSGAVARGYTSRIAGFLAPRAPQ

SEQ ID NO: 85 - protein, 207 aa, Mycobacterium avium-paratuberculosis
VGWFRFYFEGERWVWSDQVQRMHGYQPGTVTPTTELVLSHKHPADRPQVIDGINDMIRRRQAFSTRHRIVDTAGIIHHVVVVGDQLFDDSGELVGTHGFYIEVTPAATRNREDSISAKVSEIAGRRGVIDRTKGMLMLVYGIDEDAAFNMLKSLSQHGNIKLSVLAQRIAEDFTALGKEVITARSRFDQRLRTAHLRPPGAGEAGSG

SEQ ID NO: 86 - protein, 396 aa, Mycobacterium avium-paratuberculosis
VAKAKFERTKPHVNIGTIGHVDHGKTTLTAAITKVLHDKYPDLNESRAFDQIDNAPEERQRGITINISHVEYQTDKRHYAHVDAPGHADYIKNMITGAAQMDGAILVVAATDGPMPQTREHVLLARQVGVPYILVALNKADMVDDEELLELVEMEVRELLAAQEFDEDAPVVRVSALKALEGDAKWVESVEQLMEAVDESIPDPVRETDKPFLMPVEDVFTITGRGTVVTGRVERGVINVNEEVEIVGIRPSSTKTTVTGVEMFRKLLDQGQAGDNVGLLLRGIKREDVERGQVVTKPGTTTPHTEFEGQVYILSKDEGGRHTPFFNNYRPQFYFRTTDVTGVVTLPEGTEMVMPGDNTNISVKLIQPVAMDEGLRFAIREGGRTVGAGRVVKIIK

SEQ ID NO: 87 - protein, 205 aa, Mycobacterium avium-paratuberculosis
MSFVQATPEFVAAAATDLARIGSTISSANTAALGPTSGVLAPGADEVSASIAALFDAHSQVYQALSAQAAAFHSQFVQLMNGGALQYAVTEAANTTPLQSAAGPASVAAQLPAVSGAVGGSAPYGHPTAPLAAAAGASRYTRDGAGSEHPGGGTQRRGVLGTDSRPDPGQIRRGSRDEFRSRLNERHRHHPATSYGPRGTTTAKS

SEQ ID NO: 88 - protein, 331 aa, Mycobacterium avium-paratuberculosis
MRLLVTGGAGFIGANFVHSTVREHPEDSVTVLDALTYAGRRESLAGVEDSIRLVVGDITDAELVSRLVAESDAVVHFAAESHVDNALAGPEPFLHTNVVGTFTILEAVRRHGVRLHHISTDEVYGDLELDDPNRFTESTPYNPSSPYSATKAAADMLVRAWVRSYGVRATISNCSNNYGPYQHVEKFIPRQITNVLTGRRPKLYGTGANVRDWIHVDDHNSAVRRILESGEIGRTYLISSEGERDNLTVLRTLLQMMGRDPDDFDHVTDRVGHDLRYAIDPSTLYDELCWAPKHTDFEEGLRETIDWYRANESWWRPLKDASEARYEERGQ

SEQ ID NO: 89 - protein, 138 aa, Mycobacterium avium-paratuberculosis
MPPAKKAAAAPKKGQKTRRREKKNVPHGAAHIKSTFNNTIVTITDPQGNVIAWASSGHVGFKGSRKSTPFAAQLAAENAARKAQEHGVRKVDVFVKGPGSGRETAIRSLQAAGLEVGAISDVTPQPHNGVRPPKRRRV

SEQ ID NO: 90 - protein, 266 aa, Mycobacterium avium-paratuberculosis
VTVPESLDEFARTDLLLDALAQRRPVPRGQVEDPDDPDFQMLTTLLEDWRDNLRWPPASALVTPEEAVNALRAGLAERRRGHRGLAVVGSVAATLMLLSGFGAMVVEARPGSTLYGLHAMFFDQPRVNEKDQVMLAAKADLAKVAESIDKGQWDQARTQLTEVSSLVASIDDPATKQDLMTQLNLLNAKVDSRNPNATLPAAAPSMAPSVAVPAAPPPAASIAPTPAAPPAPLSPAPASTPSPSPSVGKHHHHGQPPAVAPVNPNQ

SEQ ID NO: 91 - protein, 336 aa, Mycobacterium avium-paratuberculosis
MTADTVHDVIIIGSGPAGYTAALYTARAQLAPVVFEGTSFGGALMTTTEVENYPGFRDGITGPELMDQMREQALRFGADLRMEDVESVSLAGPVKSVTTAEGETVRARAVILAMGAAARYLGVPGEQDLLGRGVSSCATCDGFFFKDQDIAVIGGGDSAMEEATFLTRFARSVTLVHRREEFRASRIMLERARANDKITIVTNKAVEAVEGSETVTGLRLRDTVTGETSTLAVTGVFVAIGHDPRSELVRDVLDTDPDGYVLVQGRTTATSIPGVFAAGDLVDRTYRQAVTAAGSGCAAAIDAERWLAEHAESSAAAQGDATEFPGSTDTLIGAPQ

SEQ ID NO: 92 - protein, 250 aa, Mycobacterium avium-paratuberculosis
VSARITPLRLEAFEQLPKHARRCVFWEVDPAVLGNHDHLADAEFEKEAWLSMVMLEWGCCGQVATAIPDERSQAEPPCLGYVFYAPPRAVPRAQRFPTGPVSADAVLLTSMGIEPGPAADDLPHALLARVIDELVRRGVRALEAFGRTPAASELQDPRLVGPDLRPVLEAVGDCSVDHCVMDAEFLKDAGFVVVAPHTYFPRLRLELDKGLGWKAEVEAALERLLESARLEQPVGAASTPANALKTAPPD

SEQ ID NO: 93 - DNA, 1925 bp, Artificial Sequence
  MAP1201c+2942c fusion nucleic acid
tctagacgctctgatgagttgggcgagttcgttctggaccacggggcagtagtaattgccgcggtcacctcgtgcacgaacacctccaaccctgaggtaatgcttggggctgcgcttctggcgcgtaacgctgtagagaagggattggcctcgaaaccatgggttaagacaacaatggctccgggatcgcaagttgtccatgactattatgacaaggcggggctgtggccttatttagaaaagctcggtttttacttagtgggctacggctgtacaacgtgtattggaaattctggtccgttaccggaagagatcagtaaagcaattaacgataatgatttatcggttaccgctgtactgagtggcaatcgcaacttcgaaggccgtatcaatccagacgttaaaatgaactaccttgcgtcgccaccattggtagtggcctatgcattggccggaacaatggattttgattttgaaaagcagccccttgggaaggacaaggatggcaatgatgtttatttgaaggatatttggcctagccagaaagatgtgagcgacacaatcgcttccgcgatcaacagcgagatgttcacaaagaactatgccgatgtattcaaaggagatgaacgctggcgtaacttacctacccctagtgggaatacatttgaatggtctccggatagcacttatgttcgtaaacccccatactttgagggaatgccggccgaacctgaaccggtagcggacatctccggcgctcgcgtcctggccttgctgggagattctgtaacaaccgatcacatttctccagcggggagcatcaaacctgggactccggcagcgcagtatttggatgaacacggcgttgatcgtaaagactacaacagttttggttcacgtcgtgggaaccatgaggtgatgattcgtggcacgttcgcaaatattcgtttacgcaaccttttattggacgatgtagcaggtggctacacacgcgattttacgcaagatggaggtccccaggcctttatttatgatgctgctcagaattatgccgcgcagaacattccgctggtggtgctggggggaaaggaatatggctcaggcagtagccgcgactgggcggcaaaaggtacgcgcctgcttggcgtccgtgcagtaattgctgagtcctttgagcgcatccatcgttccaacttaatcggtatgggtgttatccctcttcaattccctgacgggaagtccgccaaggatcttggactggacggaacggaggtattcgacatcactggcattgaagagctgaataaagggaaaacacctaaaacggtgcatgtgaaagcatcgaaaaatggaagcgacgcggtggagtttgacgccgtggttcgcattgacacgccgggcgaggcggattactaccgtaacggcggtatccttcaatacgtgttgcgcaatatgctgaagtctggccgccttcaggggatgtctcgcctgagctttgtgtgccgtctgctggctgcaaccgcctttgctgtggccctgttgcttgggttgggtgatgttccgcgcgcagcggccacagatgatcgccttcagtttacagccactaccttatcaggcgctcccttcaatggtgctagtcttcagggcaagccagctgtactttggttctggaccccctggtgtccgtactgcaatgctgaagctcccggagtcagccgcgtcgccgcagccaacccgggagtaacattcgtcggtgttgcagcgcactccgaggtgggagctatggctaatttcgtaagcaaatataacttaaactttactacgttgaacgatgctgacggcgcgatctgggcccgttatggcgttccgtggcaacctgcctatgttttttaccgtgcagatggttctagtacttttgtaaataa

SEQ ID NO: 94 - protein, 636 aa, Artificial Sequence
  MAP1201c+2942c fusion protein
SRRSDELGEFVLDHGAVVIAAVTSCTNTSNPEVMLGAALLARNAVEKGLASKPWVKTTMAPGSQVVHDYYDKAGLWPYLEKLGFYLVGYGCTTCIGNSGPLPEEISKAINDNDLSVTAVLSGNRNFEGRINPDVKMNYLASPPLVVAYALAGTMDFDFEKQPLGKDKDGNDVYLKDIWPSQKDVSDTIASAINSEMFTKNYADVFKGDERWRNLPTPSGNTFEWSPDSTYVRKPPYFEGMPAEPEPVADISGARVLALLGDSVTTDHISPAGSIKPGTPAAQYLDEHGVDRKDYNSFGSRRGNHEVMIRGTFANIRLRNLLLDDVAGGYTRDFTQDGGPQAFIYDAAQNYAAQNIPLVVLGGKEYGSGSSRDWAAKGTRLLGVRAVIAESFERIHRSNLIGMGVIPLQFPDGKSAKDLGLDGTEVFDITGIEELNKGKTPKTVHVKASKNGSDAVEFDAVVRIDTPGEADYYRNGGILQYVLRNMLKSGRLQGMSRLSFVCRLLAATAFAVALLLGLGDVPRAAATDDRLQFTATTLSGAPFNGASLQGKPAVLWFWTPWCPYCNAEAPGVSRVAAANPGVTFVGVAAHSEVGAMANFVSKYNLNFTTLNDADGAIWARYGVPWQPAYVFYRADGS

SEQ ID NO: 95 - DNA, 924 bp, Mycobacterium avium-paratuberculosis
atgacgtcggctcaaaatgagtctcaagcacttggtgatctggctgccaggcaactcgccaacgcaaccaagaccgtcccccagctctcgacgatcacgccgcgctggctgctgcacctgctgaactgggttccggtggaggcgggcatctaccgggtgaaccgggtggtcaatcccgagcaggtcgccatcaaggccgaggccggcgccggcagtgaagagccgctaccgcagacctatgtggactacgagaccagcccgcgcgagtacacgctgcgcagcatttccacgctggtcgacatccacacccgggtctccgacctgtactcgagcccgcacgatcagatcgcccagcagctgcggctgaccatcgagaccatcaaggagcgccaggagctggagctgatcaacagccccgagtatgggctgctggcccaggcgacgccggagcagacgatccagacgctggccggggctcccacgcccgacgacctcgacgcgctgatcaccaaggtgtggaagacgcccagtttcttcctgacccacccgctgggcatcgcggcgttcgggcgcgaggccacctaccggggggtgccgccgccggtggtgagcctgttcggcgcccagttcatcacctggcgcggtattccgctgatcccgtcggacaaggtgccggtggaggacggcaagacgaagttcatcctggtccgcaccggcgaggaacgtcagggcgtcgtcgggctgttccagcccggcctggtcggggagcaggcgccggggctgtcggtgcggttcaccggcatcaaccagtcggcgatcgcgacctacctggtcacgctgtacacctccctggccgtcctgaccgatgacgcgctcgccgtgctcgacgacgtcgcggtggatcagttccatgagtacaagtga

SEQ ID NO: 96 - protein, 307 aa, Mycobacterium avium-paratuberculosis
MTSAQNESQALGDLAARQLANATKTVPQLSTITPRWLLHLLNWVPVEAGIYRVNRVVNPEQVAIKAEAGAGSEEPLPQTYVDYETSPREYTLRSISTLVDIHTRVSDLYSSPHDQIAQQLRLTIETIKERQELELINSPEYGLLAQATPEQTIQTLAGAPTPDDLDALITKVWKTPSFFLTHPLGIAAFGREATYRGVPPPVVSLFGAQFITWRGIPLIPSDKVPVEDGKTKFILVRTGEERQGVVGLFQPGLVGEQAPGLSVRFTGINQSAIATYLVTLYTSLAVLTDDALAVLDDVAVDQFHEYK

SEQ ID NO: 97 - DNA, 2880 bp, Mycobacterium avium-paratuberculosis
atgttaaagttggcaccgtcgccgacacggcccgtcggcggaagaactaaatccttgggagttgaagtgactgattctgtgaactcgttcggagcccgcaacaccctcaaggtcggcgacaagagttaccagatctatcgcctcgacgccgtccccaataccgagaagcttccctacagcctcaaggtgctggccgagaacctgctgcgcaacgaggacggcagcaacatcaccaaagaccacatcgaggccatcgcgaactgggatcccaaggcggagcccagcattgaaatccagtacacgcccgcccgggtggtgatgcaggacttcaccggggtgccgtgcatcgtcgacctggccaccatgcgggaggcgatcgccgacctgggcggcaacccggagaaggtcaacccgctcgcgccggccgacctggtgatcgaccactcggtgatcgccgacctgttcggcacggccgacacgttcgagcgcaacgtcgagatcgaataccagcgcaacggcgagcgctaccagttcctgcgctgggggcagggcgccttctccgacttcaaggtggtgccgccgggcaccgggatcgtgcaccaggtgaacatcgagtacctggcccgggtggtgatggagcgcgacggggtggcctatccggacacctgcgtgggcaccgactcgcacaccacgatggtcaacggcctgggcgtgctgggctggggcgtcggcggcatcgaggccgaggccgcgatgctcggccagccggtgtcgatgctgatcccgcgggtggtcggcttcaagctgaccggtgagatccagccgggcgtgaccgccaccgacgtggtgctgaccgtcaccgagatgctgcgcaagcacggcgtggtcggcaagttcgtcgagttctacggcgagggggtggccgaggtgccgctggccaaccgcgccaccctgggcaacatgagccccgaattcggttccaccgcagcgattttcccgatcgacgaggaaaccatcgactacctgaagttcaccggccgcaacgccgagcaggtggcgctggtcgagacctacgccaaagagcagggcctgtggcacgaccccgcccacgagccggccttctcggagtacctggagctcgacctgtcccaggtggtgccctcgatcgccgggcccaagcgcccccaggaccgaattgcgttgtcgcaggccaagtccgtcttccgcgagcagatccccagctacgtcggcgacggcgacgggcagcagggctactcgaagctggacgaggtggtcgacgagacgttcccggccagcgacccgggggcgccgtccaacggccacgccgacgacctgcccgcggtgcagtcggccgccgcgcacgccaacggccgcccgagcaacccggtgacggtccgctccgacgagctgggcgagttcgtgctcgaccacggcgcggtggtgatcgccgcggtcacgtcgtgcaccaacacctccaaccccgaggtgatgctgggcgcggcgctgttggcgcgcaacgccgttgagaaggggctggcctccaagccgtgggtgaagaccacgatggcgccgggctcgcaggtggtccacgactactacgacaaggccgggctgtggccgtatttggagaagctcggcttctatctggtcggctacggctgcaccacctgcatcggcaactccggtccgctgcccgaggagatctcgaaggccatcaacgacaacgacctgtcggtgaccgcggtgctctcgggtaaccgcaacttcgagggccgcatcaacccggacgtgaagatgaactacctggcgtcgccgccgctggtggtggcctacgcgctggccggcaccatggacttcgacttcgaaaagcagccgctgggcaaggacaaggacggcaacgacgtctacctgaaggacatctggccgtcgcagaaggacgtctcggacaccatcgcatcggcgatcaactccgagatgttcaccaagaactacgccgacgtcttcaagggtgacgagcgctggcgcaacctgcccaccccgagcggcaatacctttgagtggagcccggattcgacgtacgtgcgcaagccgccgtacttcgagggcatgccggccgagcccgagccggtcgccgacatctccggcgcgcgggtgctggcgctgctgggcgactcggtgaccaccgaccacatctcccccgccggcagcatcaagccgggcaccccggcggcgcagtacctcgacgagcacggcgtggaccgcaaggattacaactccttcggctcgcggcgcggcaaccatgaggtgatgatccgcggcacgttcgccaacatccggctgcgcaacctgctgctcgacgacgtggccggcggctacacccgcgacttcacccaggacggcggtccgcaggcgttcatctacgacgcggcgcaaaactatgcggcacaaaacattccgctggtggtgctgggcggcaaggaatacgggtccggctcgtcgcgagactgggcggccaagggcacccggctgctgggcgtacgcgcggtgatcgccgagtcgttcgaacggatccaccgctccaacctgatcgggatgggtgtgatcccgctgcagttcccggacggcaaatcggccaaggacctggggctggacggcaccgaggtgttcgacatcaccggcatcgaagagctcaacaagggcaagacaccgaagacggtgcacgtcaaggcgagcaaaaacggttcggacgcagtcgaattcgatgcggtggtgcgcatcgacacccccggtgaggcggactactaccgcaacggcggcatcctgcagtacgtgctgcgcaacatgctcaagtccggctga

SEQ ID NO: 98 - protein, 959 aa, Mycobacterium avium-paratuberculosis
MLKLAPSPTRPVGGRTKSLGVEVTDSVNSFGARNTLKVGDKSYQIYRLDAVPNTEKLPYSLKVLAENLLRNEDGSNITKDHIEAIANWDPKAEPSIEIQYTPARVVMQDFTGVPCIVDLATMREAIADLGGNPEKVNPLAPADLVIDHSVIADLFGTADTFERNVEIEYQRNGERYQFLRWGQGAFSDFKVVPPGTGIVHQVNIEYLARVVMERDGVAYPDTCVGTDSHTTMVNGLGVLGWGVGGIEAEAAMLGQPVSMLIPRVVGFKLTGEIQPGVTATDVVLTVTEMLRKHGVVGKFVEFYGEGVAEVPLANRATLGNMSPEFGSTAAIFPIDEETIDYLKFTGRNAEQVALVETYAKEQGLWHDPAHEPAFSEYLELDLSQVVPSIAGPKRPQDRIALSQAKSVFREQIPSYVGDGDGQQGYSKLDEVVDETFPASDPGAPSNGHADDLPAVQSAAAHANGRPSNPVTVRSDELGEFVLDHGAVVIAAVTSCTNTSNPEVMLGAALLARNAVEKGLASKPWVKTTMAPGSQVVHDYYDKAGLWPYLEKLGFYLVGYGCTTCIGNSGPLPEEISKAINDNDLSVTAVLSGNRNFEGRINPDVKMNYLASPPLVVAYALAGTMDFDFEKQPLGKDKDGNDVYLKDIWPSQKDVSDTIASAINSEMFTKNYADVFKGDERWRNLPTPSGNTFEWSPDSTYVRKPPYFEGMPAEPEPVADISGARVLALLGDSVTTDHISPAGSIKPGTPAAQYLDEHGVDRKDYNSFGSRRGNHEVMIRGTFANIRLRNLLLDDVAGGYTRDFTQDGGPQAFIYDAAQNYAAQNIPLVVLGGKEYGSGSSRDWAAKGTRLLGVRAVIAESFERIHRSNLIGMGVIPLQFPDGKSAKDLGLDGTEVFDITGIEELNKGKTPKTVHVKASKNGSDAVEFDAVVRIDTPGEADYYRNGGILQYVLRNMLKSG

SEQ ID NO: 99 - DNA, 519 bp, Mycobacterium avium-paratuberculosis
gtgcgtcttcagggcatgtcccgtttgtcatttgtctgcaggcttttggccgcaaccgctttcgccgtcgccctgctactcgggctgggcgacgtgccgcgcgcggcggccaccgacgaccgcctgcaattcaccgcgaccacgctcagcggcgcgccgttcaacggcgccagtctgcagggcaagcccgccgtgctgtggttctggacgccgtggtgcccgtactgcaacgccgaggccccgggcgtgagccgggtggccgccgccaacccgggcgtcaccttcgtcggcgtcgccgcccactccgaagtcggcgccatggccaacttcgtctccaagtacaacctgaacttcaccacgctcaacgacgccgacggcgcgatctgggcccgctacggcgtgccctggcagcccgcgtacgtgttctaccgggcggacggcagctccaccttcgtcaacaaccccacctcggcgatgccccaggacgaactggccgcccgggtggcggcgctgcgctga

SEQ ID NO: 100 - protein, 172 aa, Mycobacterium avium-paratuberculosis; identical to SEQ ID NO: 68

SEQ ID NO: 101 - protein, 180 aa, Mycobacterium tuberculosis
VTSPHFAWLPPEINSALMFAGPGSGPLIAAATAWGELAEELLASIASLGSVTSELTSGAWLGPSAAAMMAVATQYLAWLSTAAAQAEQAAAQAMAIATAFEVALAATVQPAVVAANRGLMQLLAATNWFGQNAPALMDVEAAYEQMWALDVAAMAGYHFDASAAVAQLAPWQQVLRNLGI

SEQ ID NO: 102 - protein, 180 aa, Mycobacterium avium-paratuberculosis
MTNPHFAWLPPEVNSALIYSGPGPGPLLAAAAAWDGLAEELASSAQSFSSVTSDLASGSWQGASSAAMMTVANQYVSWLSAAAAQAEEVSHQASAIATAFEVALAATVQPAVVAANRALVQALAATNWLGQNTPAIADIEAAYEQMWASDVAAMFGYHADASAAVAKLPPWNEVLQNLGF

SEQ ID NO: 103 - protein, 177 aa, Mycobacterium tuberculosis
MEFPVLPPEINSVLMYSGAGSSPLLAAAAAWDGLAEELGSAAVSFGQVTSGLTAGVWQGAAAAAMAAAAAPYAGWLGSVAAAAEAVAGQARVVVGVFEAALAATVDPALVAANRARLVALAVSNLLGQNTPAIAAAEAEYELMWAADVAAMAGYHSGASAAAAALPAFSPPAQALGG

SEQ ID NO: 104 - protein, 177 aa, Mycobacterium tuberculosis
MNFSTLPPEINSALIFGGAGSEPMSAAAVAWDQLAMELASAAASFNSVTSGLVGESWLGPSSAAMAAAVAPYLGWLAAAAAQAQRSATQAAALVAEFEAVRAAMVQPALVAANRSDLVSLVFSNFFGQNAPAIAAIEAAYEQMWAIDVSVMSAYHAGASAVASALTPFTAPPQNLTD

SEQ ID NO: 105 - protein, 177 aa, Mycobacterium tuberculosis
MNFSVLPPEINSALIFAGAGPEPMAAAATAWDGLAMELASAAASFGSVTSGLVGGAWQGASSSAMAAAAAPYAAWLAAAAVQAEQTAAQAAAMIAEFEAVKTAVVQPMLVAANRADLVSLVMSNLFGQNAPAIAAIEATYEQMWAADVSAMSAYHAGASAIASALSPFSKPLQNLAG

SEQ ID NO: 106 - protein, 177 aa, Mycobacterium tuberculosis
MHYSVLPPEINSALIFAGAGSGPMLAAASAWDGLATELASAAVSFGSVTAGLVGGSWQGRSSVAMAAAAAPYAGWLAAAATQAEQAATQAQVMVAEFEAVRLAMVQPALVAANRSGLISLVISNLFGQNAPAIAAAEAAYEEMWALDVSAMAAYHSGASAVAVALPAFALPLRLPAG